(19) United States
(12) Patent Application Publication
(10) Pub. No.: US 2022/0363668 A1
(43) Pub. Date: Nov. 17, 2022

(54) ENZYME INHIBITORS

(71) Applicant: KALVISTA PHARMACEUTICALS LIMITED, Salisbury (GB)

(72) Inventors: Hannah Joy EDWARDS, Salisbury (GB); David Michael EVANS, Salisbury (GB); Alessandro MAZZACANI, Salisbury (GB); Alicja Stela OBARA, Salisbury (GB); David Edward CLARK, Harlow Essex (GB); Emanuela GANCIA, Harlow Essex (GB); Rachael PITTAWAY, Nottingham (GB); Joseph William WRIGGLESWORTH, Nottingham (GB)

(21) Appl. No.: 17/634,367

(22) PCT Filed: Feb. 13, 2020
(86) PCT No.: PCT/GB2020/050332

§ 371 Date: Feb. 10, 2022

(30) Foreign Application Priority Data
Aug. 21, 2019 (GB) PCT/GB2019/052359

(51) Int. Cl. C07D 401/14; C07D 401/12

(52) U.S. Cl. CPC C07D 401/14; C07D 401/12

(57) ABSTRACT

The present invention provides compounds of formula (I) compositions comprising such compounds; the use of such compounds in therapy; and methods of treating patients with such compounds; wherein R1, R2, R3, R4, R5, R6, R7, R8, R9 and R10 are as defined herein.

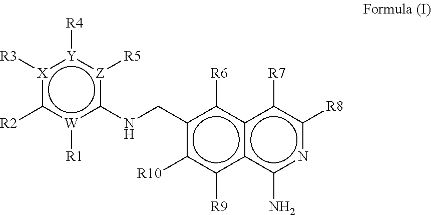

This invention relates to enzyme inhibitors that are inhibitors of Factor XIIa (FXIIa), and to the pharmaceutical compositions, and uses of, such inhibitors.

### BACKGROUND TO THE INVENTION

The compounds of the present invention are inhibitors of factor XIIa (FXIIa) and thus have a number of possible therapeutic applications, particularly in the treatment of diseases or conditions in which factor XIIa inhibition is implicated.

FXIIa is a serine protease (EC 3.4.21.38) derived from its zymogen precursor, factor XII (FXII), which is expressed by the F12 gene. Single chain FXII has a low level of amidolytic activity that is increased upon interaction with negatively charged surfaces and has been implicated in its activation (see Invanov et al., Blood. 2017 Mar. 16;129(11):1527-1537. doi: 10.1182/blood-2016-10-744110). Proteolytic cleavage of FXII to heavy and light chains of FXIIa dramatically increases catalytic activity. FXIIa that retains its full heavy chain is αFXIIa. FXIIa that retains a small fragment of its heavy chain is βFXIIa. The separate catalytic activities of αFXIIa and βFXIIa contribute to the activation and biochemical functions of FXIIa. Mutations and polymorphisms in the F12 gene can alter the cleavage of FXII and FXIIa.

FXIIa has a unique and specific structure that is different from many other serine proteases. For instance, the Tyr99 in FXIIa points towards the active site, partially blocking the S2 pocket and giving it a closed characteristic. Other serine proteases containing a Tyr99 residue (e.g. FXa, tPA and FIXa) have a more open S2 pocket. Moreover, in several trypsin-like serine proteases the P4 pocket is lined by an “aromatic box” which is responsible for the P4-driven activity and selectivity of the corresponding inhibitors. However, FXIIa has an incomplete “aromatic box” resulting in more open P4 pocket. See e.g. “Crystal structures of the recombinant β-factor XIIa protease with bound Thr-Arg and Pro-Arg substrate mimetics” M. Pathak et al., Acta. Cryst.2019, D75, 1-14; “Structures of human plasma β-factor XIIa cocrystallized with potent inhibitors” A Dementiev et al., Blood Advances 2018, 2(5), 549-558; “Design of Small-Molecule Active-Site Inhibitors of the S1A Family Proteases as Procoagulant and Anticoagulant Drugs” P. M. Fischer, J. Med. Chem., 2018, 61(9), 3799-3822; “Assessment of the protein interaction between coagulation factor XII and corn trypsin inhibitor by molecular docking and biochemical validation” B. K. Hamad et al. Journal of Thrombosis and Haemostasis, 15: 1818-1828.

FXIIa converts plasma prekallikrein (PK) to plasma kallikrein (PKa), which provides positive feedback activation of FXII to FXIIa. FXII, PK, and high molecular weight kininogen (HK) together represent the contact system. The contact system is activated via a number of mechanisms, including interactions with negatively charged surfaces, negatively charged molecules, unfolded proteins, artificial surfaces, foreign tissue (e.g. biological transplants, that include bio-prosthetic heart valves, and organ/tissue transplants), bacteria, and biological surfaces (including endothelium and extracellular matrix) that mediate assembly of contact system components. In addition, the contact system is activated by plasmin, and cleavage of FXII by other enzymes can facilitate its activation.

Activation of the contact system leads to activation of the kallikrein kinin system (KKS), complement system, and intrinsic coagulation pathway (see https://www.genome.jp/kegg-bin/show_pathway?map04610). In addition, FXIIa has additional substrates both directly, and indirectly via PKa, including Proteinase-activated receptors (PARs), plasminogen, and neuropeptide Y (NPY) which can contribute to the biological activity of FXIIa. Inhibition of FXIIa could provide clinical benefits by treating diseases and conditions associated with these systems, pathways, receptors, and hormones.

PKa activation of PAR2 mediates neuroinflammation and may contribute to neuroinflammatory disorders including multiple sclerosis (see Gob& et al., Proc Natl Acad Sci USA. 2019 Jan 2;116(1):271-276. doi: 10.1073/pnas.1810020116). PKa activation of PAR1 and PAR2 on vascular smooth muscle cells has been implicated in vascular hypertrophy and atherosclerosis (see Abdallah et al., J Biol Chem. 2010 Nov. 5; 285(45):35206-15. doi: 10.1074/jbc.M110.171769). FXIIa activation of plasminogen to plasmin contributes to fibrinolysis (see Konings et al., Thromb Res. 2015 Aug;136(2):474-80. doi: 10.1016/j.thromres.2015.06.028). PKa proteolytically cleaves NPY and thereby alters its binding to NPY receptors (Abid et al., J Biol Chem. 2009 Sep 11;284(37):24715-24. doi: 10.1074/jbc.M109.035253). Inhibition of FXIIa could provide clinical benefits by treating diseases and conditions caused by PAR signaling, NPY metabolism, and plasminogen activation.

FXIIa-mediated activation of the KKS results in the production of bradykinin (BK), which can mediate, for example, angioedema, pain, inflammation, vascular hyperpermeability, and vasodilatation (see Kaplan et al., Adv Immunol. 2014;121:41-89. doi: 10.1016/B978-0-12-800100-4.00002-7; and Hopp et al., J Neuroinflammation. 2017 Feb 20;14(1):39. doi: 10.1186/s12974-017-0815-8). CSL-312, an antibody inhibitory against FXIIa, is currently in clinical trials for the prophylactic prevention and treatment of both C1 inhibitor deficient and normal C1inhibitor hereditary angioedema (HAE), which results in intermittent swelling of face, hands, throat, gastro-intestinal tract and genitals (see https://www.clinicaltrials.gov.ct2/show/NCT03712228). Mutations in FXII that facilitate its activation to FXIIa have been identified as a cause of HAE (see Norkqvist et al., J Clin Invest. 2015 Aug. 3; 125(8):3132-46. doi: 10.1172/JC177139; and de Maat et al., J Allergy Clin Immunol. 2016 November; 138(5):1414-1423.e9. doi: 10.1016/j.jaci.2016.02.021). Since FXIIa mediates the generation of PK to PKa, inhibitors of FXIIa could provide protective effects of all form of BK-mediated angioedema, including HAE and non-hereditary bradykinin-mediated angioedema (BK-AEnH).

“Hereditary angioedema” can be defined as any disorder characterised by recurrent episodes of bradykinin-mediated angioedema (e.g. severe swelling) caused by an inherited genetic dysfunction/fault/mutation. There are currently three known categories of HAE: (i) HAE type 1, (ii) HAE type 2, and (iii) normal C1 inhibitor HAE (normal C1-Inh HAE). However, work on characterizing the etiologies of HAE is ongoing so it is expected that further types of HAE might be defined in the future.

Without wishing to be bound by theory, it is thought that HAE type 1 is caused by mutations in the SERPING1 gene that lead to reduced levels of C1 inhibitor in the blood. Without wishing to be bound by theory, it is thought that HAE type 2 is caused by mutations in the SERPING1 gene that lead to dysfunction of the C1 inhibitor in the blood. Without wishing to be bound by theory, the cause of normal C1-Inh HAE is less well defined and the underlying genetic dysfunction/fault/mutation can sometimes remain unknown. What is known is that the cause of normal C1-Inh HAE is not related to reduced levels or dysfunction of the C1 inhibitor (in contrast to HAE types 1 and 2). Normal C1-Inh HAE can be diagnosed by reviewing the family history and noting that angioedema has been inherited from a previous generation (and thus it is hereditary angioedema). Normal C1-Inh HAE can also be diagnosed by determining that there is a dysfunction/fault/mutation in a gene other than those related to C1 inhibitor. For example, it has been reported that dysfunction/fault/mutation with plasminogen can cause normal C1-Inh HAE (see e.g. Veronez et al., Front Med (Lausanne). 2019 Feb. 21; 6:28. doi: 10.3389/fmed.2019.00028; or Recke et al., Clin Transl Allergy. 2019 Feb. 14; 9:9. doi: 10.1186/s13601-019-0247-x.). It has also been reported that dysfunction/fault/mutation with Factor XII can cause normal C1-Inh HAE (see e.g. Mansi et al. 2014 The Association for the Publication of the Journal of Internal Medicine Journal of Internal Medicine, 2015, 277; 585-593; or Maat et al. J Thromb Haemost. 2019 January; 17(1):183-194. doi: 10.1111/jth.14325).

However, angioedemas are not necessarily inherited. Indeed, another class of angioedema is bradykinin mediated angioedema non-hereditary (BK-AEnH), which is not caused by an inherited genetic dysfunction/fault/mutation. Often the underlying cause of BK-AEnH is unknown and/or undefined. However, the signs and symptoms of BK-AEnH are similar to those of HAE, which, without being bound by theory, is thought to be on account of the shared bradykinin mediated pathway between HAE and BK-AEnH. Specifically, BK-AEnH is characterised by recurrent acute attacks where fluids accumulate outside of the blood vessels, blocking the normal flow of blood or lymphatic fluid and causing rapid swelling of tissues such as in the hands, feet, limbs, face, intestinal tract, airway or genitals.

Specific types of BK-AEnH include: non hereditary angioedema with normal C1 Inhibitor (AE-nC1 Inh), which can be environmental, hormonal, or drug induced; acquired angioedema; anaphylaxis associated angioedema; angiotensin converting enzyme (ACE) inhibitor induced angioedema; dipeptidyl peptidase 4 inhibitor induced angioedema; and tPA induced angioedema (tissue plasminogen activator induced angioedema). However, reasons why these factors and conditions cause angioedema in only a relatively small proportion of individuals are unknown.

Environmental factors that can induce AE-nC1 Inh include air pollution (Kedarisetty et al, Otolaryngol Head Neck Surg. 2019 April 30:194599819846446. doi: 10.1177/0194599819846446) and silver nanoparticles such as those used as antibacterial components in healthcare, biomedical and consumer products (Long et al., Nanotoxicology. 2016; 10(4):501-11. doi: 10.3109/17435390.2015.1088589).

Various publications suggest a link between the bradykinin and contact system pathways and BK-AEnHs, and also the potential efficacy of treatments, see e.g.: Bas et al. (N Engl J Med 2015; Leibfried and Kovary. J Pharm Pract 2017); van den Elzen et al. (Clinic Rev Allerg Immunol 2018); Han et al (JCI 2002).

For instance, BK-medicated AE can be caused by thrombolytic therapy. For example, tPA induced angioedema is discussed in various publications as being a potentially life threatening complication following thrombolytic therapy in acute stroke victims (see e.g. Simão et al., Blood. 2017 Apr. 20; 129(16):2280-2290. doi: 10.1182/blood-2016-09-740670; Frohlich et al., Stroke. 2019 Jun. 11:STROKEAHA119025260. doi: 10.1161/STROKEAHA.119.025260; Rathbun, Oxf Med Case Reports. 2019 Jan. 24; 2019(1):omyl12. doi: 10.1093/omcr/omyl12; Lekoubou et al., Neurol Res. 2014 July; 36(7):687-94. doi: 10.1179/1743132813Y.0000000302; Hill et al., Neurology. 2003 May 13; 60(9):1525-7).

Stone et al. (Immunol Allergy Clin North Am. 2017 August; 37(3):483-495.) reports that certain drugs can cause angioedema.

Scott et al. (Curr Diabetes Rev. 2018;14(4):327-333. doi: 10.2174/1573399813666170214113856) reports cases of dipeptidyl Peptidase-4 Inhibitor induced angioedema.

Hermanrud et al., (BMJ Case Rep. 2017 Jan. 10; 2017. pii: bcr2016217802) reports recurrent angioedema associated with pharmacological inhibition of dipeptidyl peptidase IV and also discusses acquired angioedema related to angiotensin-converting enzyme inhibitors (ACEI-AAE). Kim et al. (Basic Clin Pharmacol Toxicol. 2019 January; 124(1):115-122. doi: 10.1111/bcpt.13097) reports angiotensin II receptor blocker (ARB)-related angioedema. Reichman et al., (Pharmacoepidemiol Drug Saf. 2017 October; 26(10):1190-1196. doi: 10.1002/pds.4260) also reports angioedema risk for patients taking ACE inhibitors, ARB inhibitors and beta blockers. Diestro et al. (J Stroke Cerebrovasc Dis. 2019 May; 28(5):e44-e45. doi: 10.1016/j.jstrokecerebrovasdis.2019.01.030) also reports a possible association between certain angioedemas and ARBs.

Giard et al. (Dermatology. 2012;225(1):62-9. doi: 10.1159/000340029) reports that bradykinin mediated angioedema can be precipitated by estrogen contraception, so called “oestrogen associated angioedema”.

Contact system mediated activation of the KKS has also been implicated in retinal edema and diabetic retinopathy (see Liu et al., Biol Chem. 2013 Mar;394(3):319-28. doi: 10.1515/hsz-2012-0316). FXIIa concentrations are increased in the vitreous fluid from patients with advance diabetic retinopathy and in Diabetic Macular Edema (DME) (see Gao et al., Nat Med. 2007 February; 13(2):181-8. Epub 2007 Jan. 28 and Gao et al., J Proteome Res. 2008 June; 7(6):2516-25. doi: 10.1021/pr800112g). FXIIa has been implicated in mediating both vascular endothelial growth factor (VEGF) independent DME (see Kita et al., Diabetes. 2015 October; 64(10):3588-99. doi: 10.2337/db15-0317) and VEGF mediated DME (see Clermont et al., Invest Ophthalmol Vis Sci. 2016 May 1; 57(6):2390-9. doi: 10.1167/iovs.15-18272). FXII deficiency is protective against VEGF induced retinal edema in mice (Clermont et al., ARVO talk 2019). Therefore it has been proposed that FXIIa inhibition will provide therapeutic effects for diabetic retinopathy and retinal edema caused by retinal vascular hyperpermeability, including DME, retinal vein occlusion, age-related macular degeneration (AMD).

As noted above, the contact system can be activated by interaction with bacteria, and therefore FXIIa has been implicated in the treatment of sepsis and bacterial sepsis (see Morrison et al., J Exp Med. 1974 Sep. 1; 140(3):797-811). Therefore, FXIIa inhibitors could provide therapeutic benefits in treating sepsis, bacterial sepsis and disseminated intravascular coagulation (DIC).

FXIIa mediated activation of the KKS and production of BK have been implicated in neurodegenerative diseases including Alzheimer's disease, multiple sclerosis, epilepsy and migraine (see Zamolodchikov et al., Proc Natl Acad Sci U S A. 2015 Mar. 31; 112(13):4068-73. doi: 10.1073/pnas.1423764112; Simões et al., J Neurochem. 2019 August; 150(3):296-311. doi: 10.1111/jnc.14793; Göbel et al., Nat Commun. 2016 May 18; 7:11626. doi: 10.1038/ncomms11626; and https://clinicaltrials.gov/ct2/show/NCT03108469). Therefore, FXIIa inhibitors could provide therapeutic benefits in reducing the progression and clinical symptoms of these neurodegenerative diseases.

FXIIa has also been implicated in anaphylaxis (see Bender et al., Front Immunol. 2017 Sep. 15; 8:1115. doi: 10.3389/fimmu.2017.01115; and Sala-Cunill et al., J Allergy Clin Immunol. 2015 April; 135(4):1031-43.e6. doi: 10.1016/j.jaci.2014.07.057). Therefore, FXIIa inhibitors could provide therapeutic benefits in reducing the clinical severity and incidence of anaphylactic reactions.

The role of FXIIa in coagulation was identified over 50 years ago, and has been extensively documented in publications using biochemical, pharmacological, genetic and molecular studies (see Davie et al., Science. 1964 Sep. 18; 145(3638):1310-2). FXIIa mediated activation of factor XI (FXI) triggers the intrinsic coagulation pathway. In addition, FXIIa can increase coagulation in a FXI independent manner (see Radcliffe et al., Blood. 1977 October; 50(4):611-7; and Puy et al., J Thromb Haemost. 2013 July; 11(7):1341-52. doi: 10.1111/jth.12295). Studies on both humans and experimental animal models have demonstrated that FXII deficiency prolongs activated partial prothrombin time (APTT) without adversely affecting hemostasis (see Renné et al., J Exp Med. 2005 Jul. 18; 202(2):271-81; and Simão et al., Front Med (Lausanne). 2017 Jul. 31; 4:121. doi: 10.3389/fmed.2017.00121). Pharmacological inhibition of FXIIa also prolongs APTT without increasing bleeding (see Worm et al., Ann Transl Med. 2015 October; 3(17):247. doi: 10.3978/j.issn.2305-5839.2015.09.07). These data suggest that inhibition of FXIIa could provide therapeutic effects against thrombosis without inhibiting bleeding. Therefore, FXIIa inhibitors could be used to treat a spectrum of prothrombotic conditions including venous thromboembolism (VTE); cancer associated thrombosis; complications caused by mechanical and bioprosthetic heart valves, catheters, extracorporeal membrane oxygenation (ECMO), left ventricular assisted devices (LVAD), dialysis, cardiopulmonary bypass (CPB); sickle cell disease, joint arthroplasty, thrombosis induced by tPA, Paget-Schroetter syndrome and Budd-Chari syndrome. FXIIa inhibitor could be used for the treatment and/or prevention of thrombosis, edema, and inflammation associated with these conditions.

Surfaces of medical devices that come into contact with blood can cause thrombosis. FXIIa inhibitors may also be useful for treating or preventing thromboembolism by lowering the propensity of devices that come into contact with blood to clot blood. Examples of devices that come into contact with blood include vascular grafts, stents, in-dwelling catheters, external catheters, orthopedic prosthesis, cardiac prosthesis, and extracorporeal circulation systems.

Preclinical studies have shown that FXIIa has been shown to contribute to stroke and its complications following both ischemic stroke, and hemorrhagic accidents (see Barbieri et al., J Pharmacol Exp Ther. 2017 March; 360(3):466-475. doi: 10.1124/jpet.116.238493; Krupka et al., PLoS One. 2016 Jan. 27; 11(1):e0146783. doi: 10.1371/journal.pone.0146783; Leung et al., Trans! Stroke Res. 2012 September; 3(3):381-9. doi: 10.1007/s12975-012-0186-5; Simão et al., Blood. 2017 Apr. 20; 129(16):2280-2290. doi: 10.1182/blood-2016-09-740670; and Liu et al., Nat Med. 2011 February; 17(2):206-10. doi: 10.1038/nm.2295). Therefore, FXIIa inhibition may improve clinical neurological outcomes in the treatment of patients with stroke.

FXII deficiency has been shown to reduce the formation of atherosclerotic lesions in Apoe^{-/-} mice (Didiasova et al., Cell Signal. 2018 November; 51:257-265. doi: 10.1016/j.cellsig.2018.08.006). Therefore, FXIIa inhibitors could be used in the treatment of atherosclerosis.

FXIIa, either directly, or indirectly via PKa, has been shown to activate the complement system (Ghebrehiwet et al., Immunol Rev. 2016 November; 274(1):281-289. doi: 10.1111/imr.12469). BK increases complement C3 in the retina, and an in vitreous increase in complement C3 is associated with DME (Murugesan et al., Exp Eye Res. 2019 Jul. 24; 186:107744. doi: 10.1016/j.exer.2019.107744). Both FXIIa and PKa activate the complement system (see Irmscher et al., J Innate Immun. 2018; 10(2):94-105. doi: 10.1159/000484257; and Ghebrehiwet et al., J Exp Med. 1981 Mar. 1; 153(3):665-76).

Compounds that are said to be FXIIa inhibitors have been described by Rao et al. (“Factor XIIa Inhibitors” WO2018/093695), Hicks et al. (“Factor XIIa Inhibitors” WO2018/093716), Breslow et al. (“Aminotriazole immunomodulators for treating autoimmune diseases” WO2017/123518) and Ponda et al. (“Aminacylindazole immunomodulators for treatment of autoimmune diseases” WO2017/205296 and “Pyranopyrazole and pyrazolopyridine immunomodulators for treatment of autoimmune diseases”

WO2019/108565). FXII/FXIIa inhibitors are said to have been described by Nolte et al. (“Factor XII inhibitors for the administration with medical procedures comprising contact with artificial surfaces” WO2012/120128).

However, there remains a need to develop new FXIIa inhibitors that will have utility to treat a wide range of disorders, in particular angioedema; HAE, including: (i) HAE type 1, (ii) HAE type 2, and (iii) normal C1 inhibitor HAE (normal C1-Inh HAE); BK-AEnH, including AE-nC1 Inh, ACE and tPA induced angioedema; vascular hyperpermeability; stroke including ischemic stroke and haemorrhagic accidents; retinal edema; diabetic retinopathy; DME; retinal vein occlusion; AMD; neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; anaphylaxis; thrombosis; thromboembolism caused by increased propensity of medical devices that come into contact with blood to clot blood; prothrombotic conditions including disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; and atherosclerosis. In particular, there remains a need to develop new FXIIa inhibitors.

### DESCRIPTION OF THE INVENTION

The present invention relates to a series of amine derivatives that are inhibitors of Factor XIIa (FXIIa). The compounds of the invention are potentially useful in the treatment of diseases or conditions in which factor XIIa inhibition is implicated. The invention further relates to pharmaceutical compositions of the inhibitors, to the use of the compositions as therapeutic agents, and to methods of treatment using these composition.

The present invention provides a compound of formula (I),

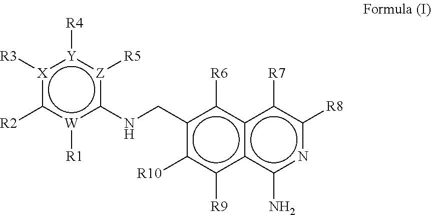

wherein:
W, X, Y, and Z are independently selected from C and N such that the ring containing W, X, Y, and Z is selected from benzene, pyridine, pyridazine, pyrimidine, pyrazine, and triazine;
R1, R4 and R5 are independently absent, or independently selected from H, alkyl, alkoxy, —CF₃, —OH, —CN, halo, —COOR12, and —CONR14R15;
when X is C, one of R2 and R3 is —L-V-R13, and the other of R2 and R3 is selected from H, alkyl, alkoxy, —CF₃, —OH, —CN, halo, —COOR12, and —CONR14R15; or
when X is N, R2 is —L-V-R13, and R3 is absent;
R6, R7, R8, R9, and R10 are independently selected from H, alkyl, alkoxy, —CF₃, —OH, —CN, halo, —COOR12, and —CONR14R15;
L is selected from a bond, alkylene, and —C(O)—;
V is absent, or selected from O and NR12;
R12 is selected from H and alkyl^{b};
R13 is (CH₂)₀₋₃(heterocyclyl);
alkyl is a linear saturated hydrocarbon having up to 4 carbon atoms (C₁-C₄) or a branched saturated hydrocarbon of 3 or 4 carbon atoms (C₃-C₄); alkyl may optionally be substituted with 1 or 2 substituents independently selected from (C₁-C₃)alkoxy, —OH, —CN, —NR14R15, —NHCOCH₃, halo, —COOR12, and —CONR14R15;
alkyl^{b} is a linear saturated hydrocarbon having up to 4 carbon atoms (C₁-C₄) or a branched saturated hydrocarbon of 3 or 4 carbon atoms (C₃-C₄); alkyl^{b} may optionally be substituted with 1 or 2 substituents independently selected from —OH, —CN, —NHCOCH₃, and halo;
alkylene is a bivalent linear saturated hydrocarbon having 1 to 4 carbon atoms (C₁-C₄) or a branched bivalent saturated hydrocarbon having 3 to 4 carbon atoms (C₃-C₄);
alkoxy is a linear O-linked hydrocarbon of between 1 and 3 carbon atoms (C₁-C₃) or a branched O-linked hydrocarbon of between 3 and 4 carbon atoms (C₃-C₄); alkoxy may optionally be substituted with 1 or 2 substituents independently selected from —OH, —CN, —CF₃, —N(R12)₂ and fluoro;
halo is F, CI, Br, or I;
heterocyclyl is a 4-, 5-, or 6-, membered carbon-containing non-aromatic ring containing one or two ring members that are selected from N, NR16, and O; heterocyclyl may be optionally substituted with 1, 2, 3, or 4 substituents independently selected from alkyl, alkoxy, oxo, —CF₃, —OH, —CN, halo, —COOR12, and —CONR14R15;
R14 and R15 are independently selected from H, and alkyl^{b};
R16 is selected from H, and alkyl;
and tautomers, isomers, stereoisomers (including enantiomers, diastereoisomers and racemic and scalemic mixtures thereof), deuterated isotopes, and pharmaceutically acceptable salts and/or solvates thereof.

The compounds of the present invention have been developed to be inhibitors of FXIIa. As noted above, FXIIa has a unique and specific binding site and there is a need for small molecule FXIIa inhibitors.

The present invention also provides a prodrug of a compound as herein defined, or a pharmaceutically acceptable salt and/or solvate thereof.

The present invention also provides an N-oxide of a compound as herein defined, or a prodrug or pharmaceutically acceptable salt and/or solvate thereof.

It will be understood that certain compounds of the present invention may exist in solvated, for example hydrated, as well as unsolvated forms. It is to be understood that the present invention encompasses all such solvated forms.

It will be understood that “pharmaceutically acceptable salts and/or solvates thereof” means “pharmaceutically acceptable salts thereof”, “pharmaceutically acceptable solvates thereof”, and “pharmaceutically acceptable solvates of salts thereof”.

It will be understood that substituents may be named as its free unbonded structure (e.g. piperidine) or by its bonded structure (e.g. piperidinyl). No difference is intended.

It will be understood that the compounds of the invention comprise several substituents. When any of these substituents is defined more specifically herein, the substituents/optional substituents to these groups described above also apply, unless stated otherwise. For example, R13 can be —(CH₂)₀₋₃ heterocyclyl, which more specifically can be piperidinyl. In this case, piperidinyl can be optionally substituted in the same manner as “heterocyclyl”.

It will be understood that “alkylene” has two free valencies i.e. it is bivalent, meaning that it is capable of being bonded to twice. For example, when two adjacent ring atoms on A″ are linked by an alkylene to form a cyclopentane, the alkylene will be —CH₂CH₂CH₂—.

It will be understood that when any variable (e.g. alkyl) occurs more than once, its definition on each occurrence is independent of every other occurrence.

It will be understood that combinations of substituents and variables are permissible only if such combinations result in stable compounds.

As used herein the term “bradykinin-mediated angioedema” means hereditary angioedema, and any non-hereditary bradykinin-mediated angioedema. For example, “bradykinin-mediated angioedema” encompasses hereditary angioedema and acute bradykinin-mediated angioedema of unknown origin.

As used herein, the term “hereditary angioedema” means any bradykinin-mediated angioedema caused by an inherited genetic dysfunction, fault, or mutation. As a result, the term “HAE” includes at least HAE type 1, HAE type 2, and normal C1 inhibitor HAE (normal C1-Inh HAE).

The invention is also described by the appended numbered embodiments.

As noted above, in Formula (I), W, X, Y, and Z are independently selected from C and N such that the ring containing W, X, Y, and Z is selected from benzene, pyridine, pyridazine, pyrimidine, pyrazine, and triazine.

W, X, Y and Z may be independently selected from C and N such that the ring containing W, X, Y and Z is selected from benzene, pyridine and pyrazine.

In particular, W, X, Y and Z may each independently be C so that the ring containing W, X, Y and Z is benzene.

Alternatively, W, X, Y and Z are independently selected from C and N such that the ring containing W, X, Y and Z is pyridine.

Alternatively, W, X, Y and Z are independently selected from C and N such that the ring containing W, X, Y and Z is pyridazine.

Alternatively, W, X, Y and Z are independently selected from C and N such that the ring containing W, X, Y and Z is pyrimidine.

Alternatively, W, X, Y and Z are independently selected from C and N such that the ring containing W, X, Y and Z is pyrazine.

Alternatively, W, X, Y and Z are independently selected from C and N such that the ring containing W, X, Y and Z is triazine.

Where W is N, R1 is absent.

Where X is N, R3 is absent.

Where Y is N, R4 is absent.

Where Z is N, R5 is absent

In combination with the possible options for W, X, Y Z described above, R1, R4 and R5 are independently absent, or independently selected from H, alkyl, alkoxy, —CF₃, —OH, —CN, halo, —COOR12, and —CONR14R15.

As described above where W is N, R1 is absent. Alternatively, where W is C, R1 may be selected from H, alkyl, alkoxy, —CF₃, —OH, —CN, halo, —COOR12, and —CONR14R15.

R1 may be H. Alternatively, R1 may be alkyl, in particular methyl or ethyl. Alternatively, R1 may be alkoxy, in particular methoxy. Alternatively, R1 may be —OH. Alternatively, R1 may be —CF₃. Alternatively R1 may be —CN. Alternatively R1 may be halo, in particular CI or F. Alternatively, R1 may be —COOR12. Alternatively, R1 may be—CONR14R15, in particular, —CONH₂.

As described above, where Y is N, R4 is absent. Alternatively, where Y is C, R4 may be selected from H, alkyl, alkoxy, —CF₃, —OH, —CN, halo, —COOR12, and —CONR14R15.

R4 may be H. Alternatively, R4 may be alkyl, in particular methyl or ethyl. Alternatively, R4 may be alkoxy, in particular methoxy. Alternatively, R4 may be —OH. Alternatively, R4 may be —CF₃. Alternatively R4 may be —CN. Alternatively R4 may be halo, in particular CI or F. Alternatively, R4 may be —COOR12. Alternatively, R4 may be—CONR14R15, in particular, —CONH₂.

As described above, where Z is N, R4 is absent. Alternatively, where Z is C, R5 may be selected from H, alkyl, alkoxy, —CF₃, —OH, —CN, halo, —COOR12, and —CONR14R15.

R5 may be H. Alternatively, R5 may be alkyl, in particular methyl or ethyl. Alternatively, R5 may be alkoxy, in particular methoxy. Alternatively, R5 may be —OH. Alternatively, R5 may be —CF₃. Alternatively R5 may be —CN. Alternatively R5 may be halo, in particular CI or F. Alternatively, R5 may be —COOR12. Alternatively, R5 may be—CONR14R15, in particular, —CONH₂.

Preferably, R1, R4 and R5 are independently absent or H.

In combination with the definitions above for W, X, Y, Z, R1, R4 and R5, when X is C, one of R2 and R3 is —L-V-R13, and the other of R2 and R3 is selected from H, alkyl, alkoxy, —CF₃, —OH, —CN, halo, —COOR12, and —CONR14R15; or
when X is N, R2 is —L-V-R13, and R3 is absent;

In one embodiment, X is C, R2 is selected from H, alkyl, alkoxy, —CF₃, —OH, —CN, halo, —COOR12, and —CONR14R15 and R3 is —L-V-R13. In such an embodiment, R2 may be H or alkyl, in particular methyl or ethyl. Preferably, R2 is H.

Alternatively, X is C, R2 is —L-V-R13 and R3 is selected from H, alkyl, alkoxy, —CF₃, —OH, —CN, halo, —COOR12, and —CONR14R15 and R3 is —L-V-R13. In such an embodiment, R3 may be H or alkyl, in particular methyl or ethyl. Preferably, R2 is H.

Preferably, when X is C, R2 is selected from H, alkyl, alkoxy, —CF₃, —OH, —CN, halo, —COOR12, and —CONR14R15 and R3 is —L-V-R13. In such preferred embodiments, R2 is preferably H.

In the group —L-V-R13, L is selected from a bond, alkylene, and —C(O)—; V is absent, or selected from O and NR12, wherein R12 is selected from H and alkyl^{b}; and R13 is (CH₂)₀₋₃ (heterocyclyl).

Preferably L is a bond or methylene.

Preferably V is absent or O.

R13 is (CH₂)₀₋₃ (heterocyclyl), preferably CH₂ (heterocyclyl) or -(heterocyclyl).

Preferred heterocyclyl groups include a 6-membered carbon-containing non-aromatic ring containing one or two ring members that are selected from N, NR16, and O; heterocyclyl may be optionally substituted with 1, 2, 3, or 4 substituents independently selected from alkyl, alkoxy, oxo, —CF₃, —OH, —CN, halo, —COOR12, and —CONR14R15. Preferably, heterocyclyl is piperidinyl i.e. a 6-membered carbon-containing non-aromatic containing one NR16. Preferably, R16 is CH₃.

In some embodiments, L is a bond, V is O and R13 is CH₂ (heterocyclyl) wherein heterocyclyl may be substituted as defined above, preferably wherein heterocyclyl is a 6-membered carbon-containing non-aromatic ring containing NR16 wherein R16 is alkyl, preferably methyl. In particular, it is preferred that the heterocyclyl on R13 is piperidinyl, which may be optionally substituted as for heterocyclyl, preferably wherein the N atom of the piperidinyl is substituted with an alkyl group, preferably methyl.

In alternative embodiments, L is a bond, V is O and R13 is heterocyclyl wherein heterocyclyl may be substituted as defined above, preferably wherein heterocyclyl is a 6-membered carbon-containing non-aromatic ring containing NR16 wherein R16 is alkyl, preferably methyl. In particular, it is preferred that the heterocyclyl on R13 is piperidinyl, which may be optionally substituted as for heterocyclyl, preferably wherein the N atom of the piperidinyl is substituted with an alkyl group, preferably methyl.

In further embodiments, L is alkylene, preferably methylene, V is O and R13 is heterocyclyl wherein heterocyclyl may be substituted as defined above, preferably wherein heterocyclyl is a 6-membered carbon-containing non-aromatic ring containing NR16 wherein R16 is alkyl, preferably methyl. In particular, it is preferred that the heterocyclyl on R13 is piperidinyl, which may be optionally substituted as for heterocyclyl, preferably wherein the N atom of the piperidinyl is substituted with an alkyl group, preferably methyl.

As described above, R6, R7, R8, R9, and R10 are independently selected from H, alkyl, alkoxy, —CF₃, —OH, —CN, halo, —COOR12, and —CONR14R15.

Preferably R6, R7, R8, R9, and R10 are independently selected from H and alkyl, preferably H.

In one embodiment, R6, R7, R8, R9 and R10 are all the same and are all H.

R14 and R15 are independently selected from H, and alkyl^{b}. R14 and R15 may be the same or different.

In one embodiment, R14 and R15 are the same and are H.

R16 is selected from H, and alkyl. Preferably R16 is alkyl, in particular, —CH₃.

W, X, Y and Z may be independently selected from C and N such that the ring containing W, X, Y and Z is selected from benzene, pyridine and pyrazine; R1, R4 and R5 are independently absent or H; X is C; R2 is selected from H, alkyl, alkoxy, —CF₃, —OH, —CN, halo, —COOR12, and —CONR14R15, preferably H or alkyl, more preferably H; R3 is —L-V-R13, wherein L is a bond; V is O and R13 is CH₂ (heterocyclyl) wherein heterocyclyl may be substituted as defined above, preferably wherein heterocyclyl is a 6-membered carbon-containing non-aromatic ring containing NR16 wherein R16 is alkyl, preferably methyl; and wherein R6, R7, R8, R9 and R10 are all the same and are all H. In particular, it is preferred that the heterocyclyl on R13 is piperidinyl, which may be optionally substituted as for heterocyclyl, preferably wherein the N atom of the piperidinyl is substituted with an alkyl group, preferably methyl.

W, X, Y and Z may be independently selected from C and N such that the ring containing W, X, Y and Z is selected from benzene, pyridine and pyrazine; R1, R4 and R5 are independently absent or H; X is C; R2 is selected from H, alkyl, alkoxy, —CF₃, —OH, —CN, halo, —COOR12, and —CONR14R15, preferably H or alkyl, preferably H; R3 is —L-V-R13, wherein L is a bond, V is O and R13 is heterocyclyl wherein heterocyclyl may be substituted as defined above, preferably wherein heterocyclyl is a 6-membered carbon-containing non-aromatic ring containing NR16 wherein R16 is alkyl, preferably methyl; and wherein R6, R7, R8, R9 and R10 are all the same and are all H. In particular, it is preferred that the heterocyclyl on R13 is piperidinyl, which may be optionally substituted as for heterocyclyl, preferably wherein the N atom of the piperidinyl is substituted with an alkyl group, preferably methyl.

W, X, Y and Z may be independently selected from C and N such that the ring containing W, X, Y and Z is selected from benzene, pyridine and pyrazine; R1, R4 and R5 are independently absent or H; X is C; R2 is selected from H, alkyl, alkoxy, —CF₃,—OH, —CN, halo, —COOR12, and —CONR14R15, preferably H or alkyl, preferably H; and R3 is —L-V-R13, wherein L is alkylene, preferably methylene, V is O and R13 is heterocyclyl wherein heterocyclyl may be substituted as defined above, preferably wherein heterocyclyl is a 6-membered carbon-containing non-aromatic ring containing NR16 wherein R16 is alkyl, preferably methyl; and wherein R6, R7, R8, R9 and R10 are all the same and are all H. In particular, it is preferred that the heterocyclyl on R13 is piperidinyl, which may be optionally substituted as for heterocyclyl, preferably wherein the N atom of the piperidinyl is substituted with an alkyl group, preferably methyl.

Preferably, W, X, Y and Z may be independently selected from C and N such that the ring containing W, X, Y and Z is benzene; R1, R4 and R5 are all H; X is C; R2 is selected from H, alkyl, alkoxy, —CF₃, —OH, —CN, halo, —COOR12, and —CONR14R15, preferably H or alkyl, preferably H; and R3 is —L-V-R13, wherein L is a bond, V is O and R13 is heterocyclyl wherein heterocyclyl may be substituted as defined above, preferably wherein heterocyclyl is a 6-membered carbon-containing non-aromatic ring containing NR16 wherein R16 is alkyl, preferably methyl; and wherein R6, R7, R8, R9 and R10 are all the same and are all H. In particular, it is preferred that the heterocyclyl on R13 is piperidinyl, which may be optionally substituted as for heterocyclyl, preferably wherein the N atom of the piperidinyl is substituted with an alkyl group, preferably methyl.

Preferably, W, X, Y and Z may be independently selected from C and N such that the ring containing W, X, Y and Z is pyridine; R1, R4 and R5 are independently absent or H; X is C; R2 is selected from H, alkyl, alkoxy, —CF₃, —OH, —CN, halo, —COOR12, and —CONR14R15, preferably H or alkyl, preferably H; R3 is -L-V-R13, wherein L is a bond, V is O and R13 is heterocyclyl wherein heterocyclyl may be substituted as defined above, preferably wherein heterocyclyl is a 6-membered carbon-containing non-aromatic ring containing NR16 wherein R16 is alkyl, preferably methyl; and wherein R6, R7, R8, R9 and R10 are all the same and are all H. In particular, it is preferred that the heterocyclyl on R13 is piperidinyl, which may be optionally substituted as for heterocyclyl, preferably wherein the N atom of the piperidinyl is substituted with an alkyl group, preferably methyl.

Preferably, W, X, Y and Z may be independently selected from C and N such that the ring containing W, X, Y and Z is pyridine; R1, R4 and R5 are independently absent or H; X is C; R2 is selected from H, alkyl, alkoxy, —CF₃, —OH, —CN, halo, —COOR12, and —CONR14R15, preferably H or alkyl, preferably H; R3 is —L-V-R13, wherein L is alkylene, preferably methylene, V is O and R13 is heterocyclyl wherein heterocyclyl may be substituted as defined above, preferably wherein heterocyclyl is a 6-membered carbon-containing non-aromatic ring containing NR16 wherein R16 is alkyl, preferably methyl; and wherein R6, R7, R8, R9 and R10 are all the same and are all H. In particular, it is preferred that the heterocyclyl on R13 is piperidinyl, which may be optionally substituted as for heterocyclyl, preferably wherein the N atom of the piperidinyl is substituted with an alkyl group, preferably methyl.

Preferably, W, X, Y and Z may be independently selected from C and N such that the ring containing W, X, Y and Z is pyridine; R1, R4 and R5 are independently absent or H; X is C; R2 is selected from H, alkyl, alkoxy, —CF₃, —OH, —CN, halo, —COOR12, and —CONR14R15, preferably H or alkyl, preferably H; R3 is —L-V-R13, wherein L is a bond, V is O and R13 is CH₂ (heterocyclyl) wherein heterocyclyl may be substituted as defined above, preferably wherein heterocyclyl is a 6-membered carbon-containing non-aromatic ring containing NR16 wherein R16 is alkyl, preferably methyl; and wherein R6, R7, R8, R9 and R10 are all the same and are all H. In particular, it is preferred that the heterocyclyl on R13 is piperidinyl, which may be optionally substituted as for heterocyclyl, preferably wherein the N atom of the piperidinyl is substituted with an alkyl group, preferably methyl.

Preferably, W, X, Y and Z may be independently selected from C and N such that the ring containing W, X, Y and Z is pyridine; R1, R4 and R5 are independently absent or H; X is C; R3 is selected from H, alkyl, alkoxy, —CF₃, —OH, —CN, halo, —COOR12, and —CONR14R15, preferably H or alkyl, preferably H; R2 is —L-V-R13, wherein L is a bond, V is O and R13 is heterocyclyl wherein heterocyclyl may be substituted as defined above, preferably wherein heterocyclyl is a 6-membered carbon-containing non-aromatic ring containing NR16 wherein R16 is alkyl, preferably methyl and wherein R6, R7, R8, R9 and R10 are all the same and are all H. In particular, it is preferred that the heterocyclyl on R13 is piperidinyl, which may be optionally substituted as for heterocyclyl, preferably wherein the N atom of the piperidinyl is substituted with an alkyl group, preferably methyl.

Preferably, W, X, Y and Z may be independently selected from C and N such that the ring containing W, X, Y and Z is pyrazine; R1, R4 and R5 are independently absent or H; X is C; R2 is selected from H, alkyl, alkoxy,—CF₃, —OH, —CN, halo, —COOR12, and —CONR14R15, preferably H or alkyl, preferably H; R3 is —L-V-R13, wherein L is a bond, V is O and R13 is CH₂ (heterocyclyl) wherein heterocyclyl may be substituted as defined above, preferably wherein heterocyclyl is a 6-membered carbon-containing non-aromatic ring containing NR16 wherein R16 is alkyl, preferably methyl and wherein R6, R7, R8, R9 and R10 are all the same and are all H. In particular, it is preferred that the heterocyclyl on R13 is piperidinyl, which may be optionally substituted as for heterocyclyl, preferably wherein the N atom of the piperidinyl is substituted with an alkyl group, preferably methyl.

Preferably, W, X, Y and Z may be independently selected from C and N such that the ring containing W, X, Y and Z is pyrazine; R1, R4 and R5 are independently absent or H; X is C; R2 is selected from H, alkyl, alkoxy, —CF₃, —OH, —CN, halo, —COOR12, and —CONR14R15, preferably H or alkyl, preferably H; R3 is —L-V-R13, wherein L is a bond, V is O and R13 is heterocyclyl wherein heterocyclyl may be substituted as defined above, preferably wherein heterocyclyl is a 6-membered carbon-containing non-aromatic ring containing NR16 wherein R16 is alkyl, preferably methyl; and wherein R6, R7, R8, R9 and R10 are all the same and are all H. In particular, it is preferred that the heterocyclyl on R13 is piperidinyl, which may be optionally substituted as for heterocyclyl, preferably wherein the N atom of the piperidinyl is substituted with an alkyl group, preferably methyl.

The present invention also encompasses, but is not limited to, the compounds below in Table 1 or Table 2, and pharmaceutically acceptable salts and/or solvates thereof.

The compounds of the invention can be selected from Table 1, and pharmaceutically acceptable salts and/or solvates thereof.

The compounds of the invention can be selected from Table 2, and pharmaceutically acceptable salts and/or solvates thereof.

TABLE 1

| Structure | |
|---|---|
| Molecular formula | Example No. |
| 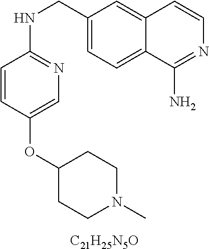 [SMILES: CN1CCC(Oc2ccc(NCc3ccc4c(N)nccc4c3)nc2)CC1 ; "C21H25N5O"] | 18.01 |
| 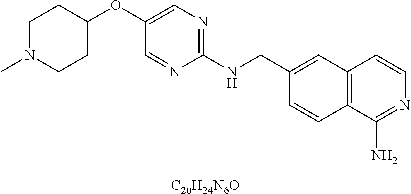 [SMILES: CN1CCC(Oc2cnc(NCc3ccc4c(N)nccc4c3)nc2)CC1 ; "C20H24N6O"] | 18.02 |
| 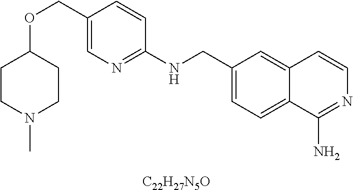 [SMILES: CN1CCC(OCc2ccc(NCc3ccc4c(N)nccc4c3)nc2)CC1 ; "C22H27N5O"] | 18.03 |
| 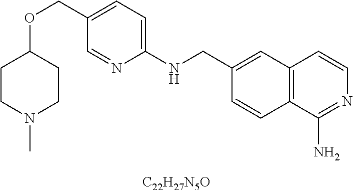 [SMILES: CN1CCC(OCc2ccc(NCc3ccc4c(N)nccc4c3)nc2)CC1 ; "C22H27N5O"] | 18.04 |
| 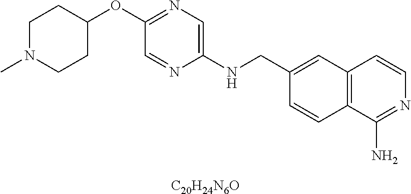 [SMILES: CN1CCC(Oc2cnc(NCc3ccc4c(N)nccc4c3)cn2)CC1 ; "C20H24N6O"] | 18.05 |
| 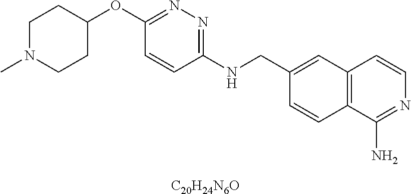 [SMILES: CN1CCC(Oc2ccc(NCc3ccc4c(N)nccc4c3)nn2)CC1 ; "C20H24N6O"] | 18.06 |
| 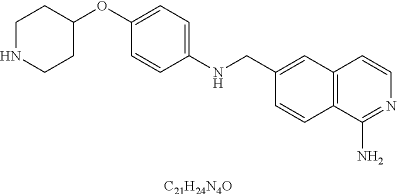 [SMILES: Nc1nccc2cc(CNc3ccc(OC4CCNCC4)cc3)ccc12 ; "C21H24N4O"] | 18.07 |
| 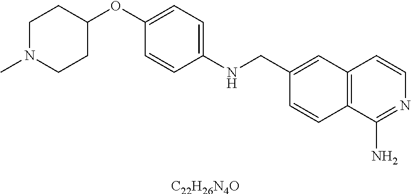 [SMILES: CN1CCC(Oc2ccc(NCc3ccc4c(N)nccc4c3)cc2)CC1 ; "C22H26N4O"] | 18.08 |
| 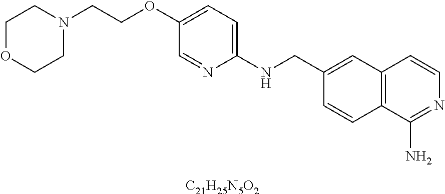 [SMILES: Nc1nccc2cc(CNc3ccc(OCCN4CCOCC4)cn3)ccc12 ; "C21H25N5O2"] | 18.09 |
| 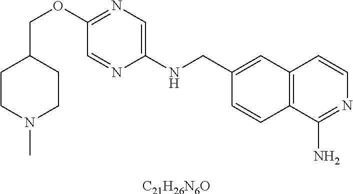 [SMILES: CN1CCC(COc2cnc(NCc3ccc4c(N)nccc4c3)cn2)CC1 ; "C21H26N6O"] | 18.10 |
| 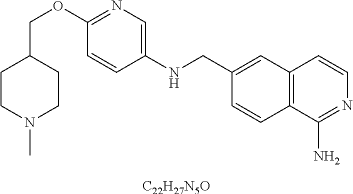 [SMILES: CN1CCC(COc2ccc(NCc3ccc4c(N)nccc4c3)cn2)CC1 ; "C22H27N5O"] | 18.11 |
| 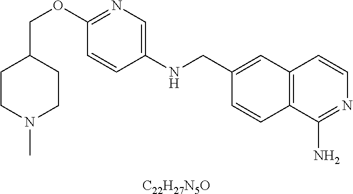 [SMILES: CN1CCC(COc2ccc(NCc3ccc4c(N)nccc4c3)cn2)CC1 ; "C22H27N5O"] | 18.12 |
| 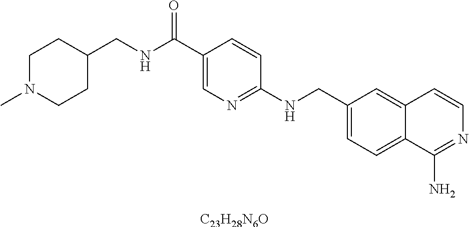 [SMILES: CN1CCC(CNC(=O)c2ccc(NCc3ccc4c(N)nccc4c3)nc2)CC1 ; "C23H28N6O"] | 18.13 |
| 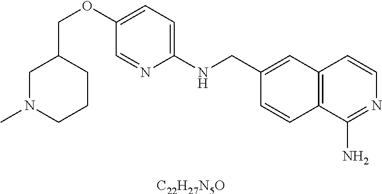 [SMILES: CN1CCCC(COc2ccc(NCc3ccc4c(N)nccc4c3)nc2)C1 ; "C22H27N5O"] | 18.14 |
| 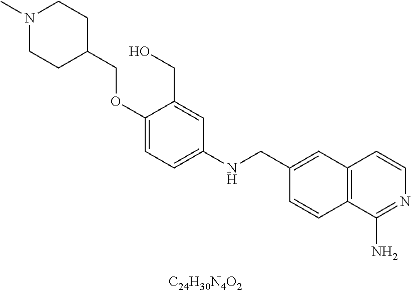 [SMILES: CN1CCC(COc2ccc(NCc3ccc4c(N)nccc4c3)cc2CO)CC1 ; "C24H30N4O2"] | 18.15 |
| 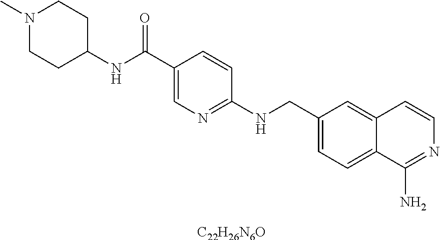 [SMILES: CN1CCC(NC(=O)c2ccc(NCc3ccc4c(N)nccc4c3)nc2)CC1 ; "C22H26N6O"] | 18.16 |
| 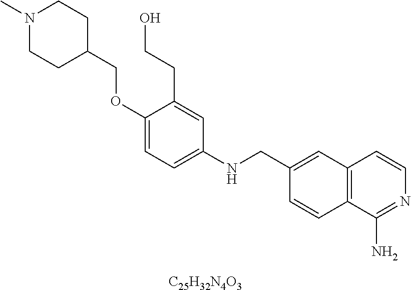 [SMILES: CN1CCC(COc2ccc(NCc3ccc4c(N)nccc4c3)cc2CCO)CC1 ; "C25H32N4O3"] | 18.17 |
| 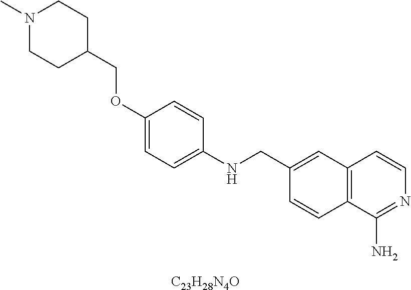 [SMILES: CN1CCC(COc2ccc(NCc3ccc4c(N)nccc4c3)cc2)CC1 ; "C23H28N4O"] | 18.18 |
| 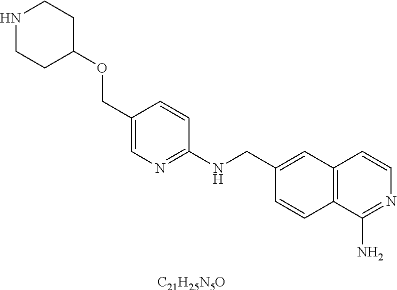 [SMILES: Nc1nccc2cc(CNc3ccc(COC4CCNCC4)cn3)ccc12 ; "C21H25N5O"] | 18.19 |
| 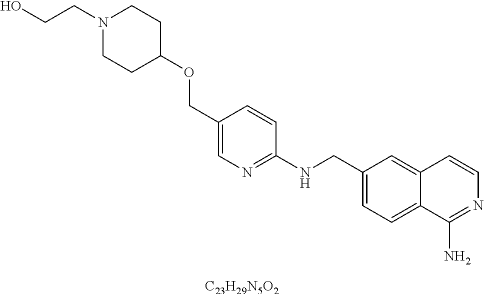 [SMILES: Nc1nccc2cc(CNc3ccc(COC4CCN(CCO)CC4)cn3)ccc12 ; "C23H29N5O2"] | 18.20 |
| 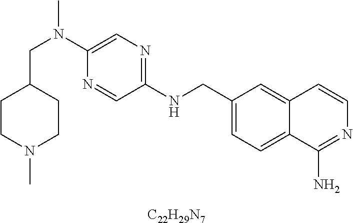 [SMILES: CN1CCC(CN(C)c2cnc(NCc3ccc4c(N)nccc4c3)cn2)CC1 ; "C22H29N7"] | 18.21 |
| 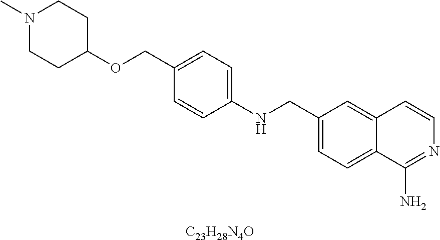 [SMILES: CN1CCC(OCc2ccc(NCc3ccc4c(N)nccc4c3)cc2)CC1 ; "C23H28N4O"] | 18.22 |
| 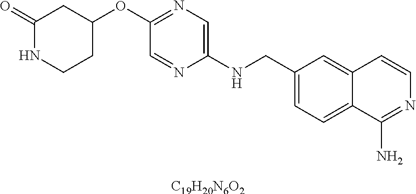 [SMILES: Nc1nccc2cc(CNc3cnc(OC4CCNC(=O)C4)cn3)ccc12 ; "C19H20N6O2"] | 18.23 |
| 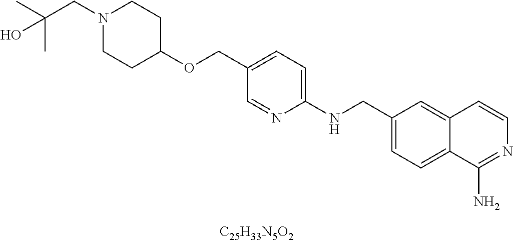 [SMILES: CC(C)(O)CN1CCC(OCc2ccc(NCc3ccc4c(N)nccc4c3)nc2)CC1 ; "C25H33N5O2"] | 18.24 |

TABLE 2

| Structure | |
|---|---|
| Molecular formula | Example No. |
| 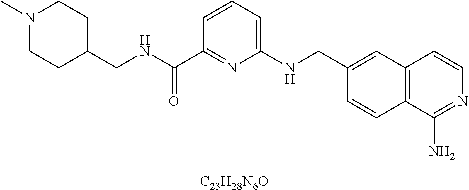 [SMILES: CN1CCC(CNC(=O)c2cccc(NCc3ccc4c(N)nccc4c3)n2)CC1 ; "C23H28N6O"] | 18.201 |
| 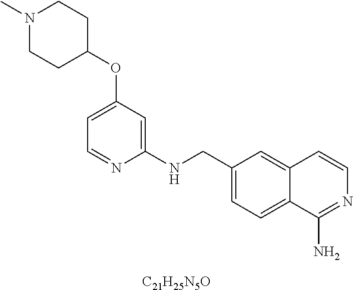 [SMILES: CN1CCC(Oc2ccnc(NCc3ccc4c(N)nccc4c3)c2)CC1 ; "C21H25N5O"] | 18.202 |
| 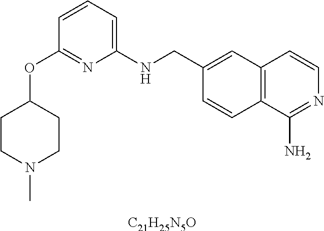 [SMILES: CN1CCC(Oc2cccc(NCc3ccc4c(N)nccc4c3)n2)CC1 ; "C21H25N5O"] | 18.203 |
| 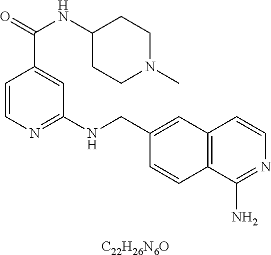 [SMILES: CN1CCC(NC(=O)c2ccnc(NCc3ccc4c(N)nccc4c3)c2)CC1 ; "C22H26N6O"] | 18.204 |
| 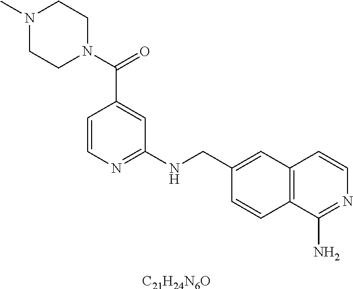 [SMILES: CN1CCN(C(=O)c2ccnc(NCc3ccc4c(N)nccc4c3)c2)CC1 ; "C21H24N6O"] | 18.205 |
| 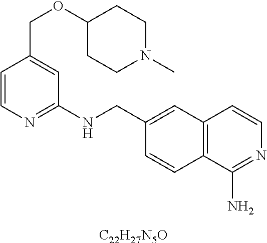 [SMILES: CN1CCC(OCc2ccnc(NCc3ccc4c(N)nccc4c3)c2)CC1 ; "C22H27N5O"] | 18.206 |
| 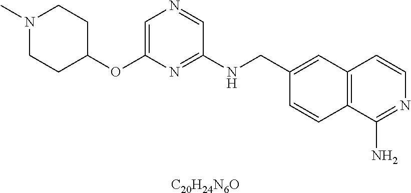 [SMILES: CN1CCC(Oc2cncc(NCc3ccc4c(N)nccc4c3)n2)CC1 ; "C20H24N6O"] | 18.207 |
| 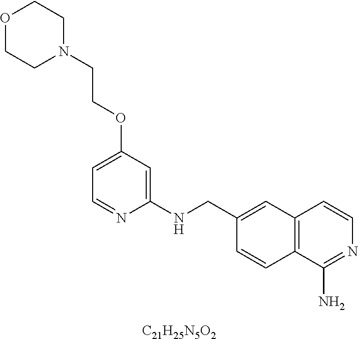 [SMILES: Nc1nccc2cc(CNc3cc(OCCN4CCOCC4)ccn3)ccc12 ; "C21H25N5O2"] | 18.208 |
| 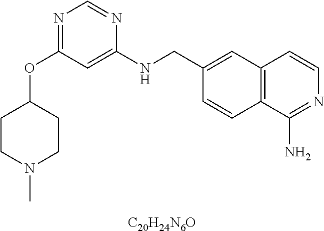 [SMILES: CN1CCC(Oc2cc(NCc3ccc4c(N)nccc4c3)ncn2)CC1 ; "C20H24N6O"] | 18.209 |
| 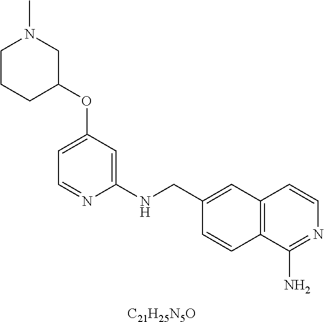 [SMILES: CN1CCCC(Oc2ccnc(NCc3ccc4c(N)nccc4c3)c2)C1 ; "C21H25N5O"] | 18.210 |
| 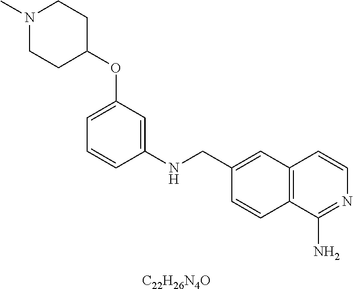 [SMILES: CN1CCC(Oc2cccc(NCc3ccc4c(N)nccc4c3)c2)CC1 ; "C22H26N4O"] | 18.211 |
| 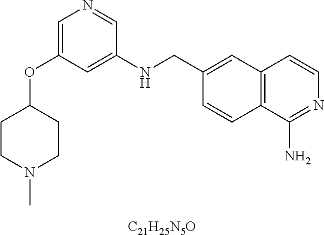 [SMILES: CN1CCC(Oc2cncc(NCc3ccc4c(N)nccc4c3)c2)CC1 ; "C21H25N5O"] | 18.212 |
| 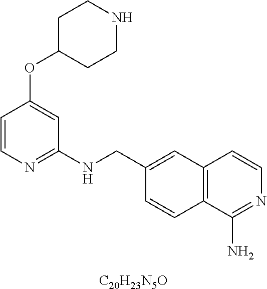 [SMILES: Nc1nccc2cc(CNc3cc(OC4CCNCC4)ccn3)ccc12 ; "C20H23N5O"] | 18.213 |
| 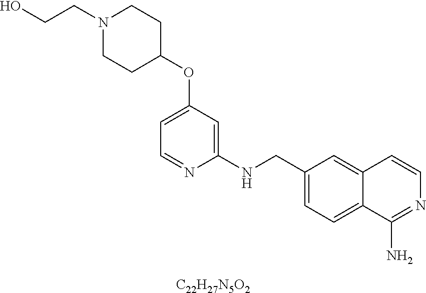 [SMILES: Nc1nccc2cc(CNc3cc(OC4CCN(CCO)CC4)ccn3)ccc12 ; "C22H27N5O2"] | 18.214 |
| 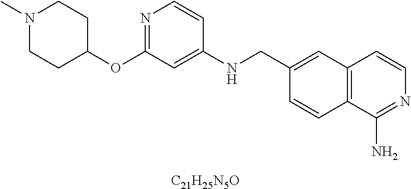 [SMILES: CN1CCC(Oc2cc(NCc3ccc4c(N)nccc4c3)ccn2)CC1 ; "C21H25N5O"] | 18.215 |
| 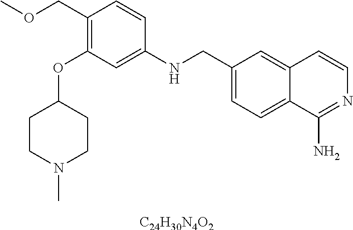 [SMILES: COCc1ccc(NCc2ccc3c(N)nccc3c2)cc1OC1CCN(C)CC1 ; "C24H30N4O2"] | 18.216 |
| 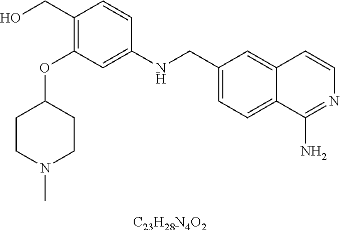 [SMILES: CN1CCC(Oc2cc(NCc3ccc4c(N)nccc4c3)ccc2CO)CC1 ; "C23H28N4O2"] | 18.217 |
| 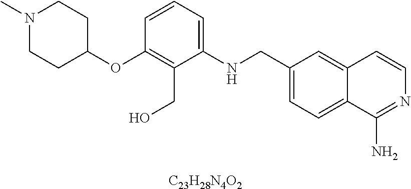 [SMILES: CN1CCC(Oc2cccc(NCc3ccc4c(N)nccc4c3)c2CO)CC1 ; "C23H28N4O2"] | 18.218 |
| 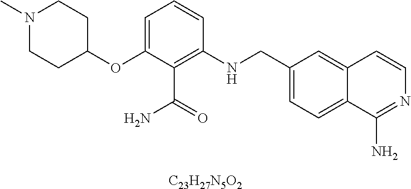 [SMILES: CN1CCC(Oc2cccc(NCc3ccc4c(N)nccc4c3)c2C(N)=O)CC1 ; "C23H27N5O2"] | 18.219 |

Preferably, the compound of formula (I) is a compound selected from:

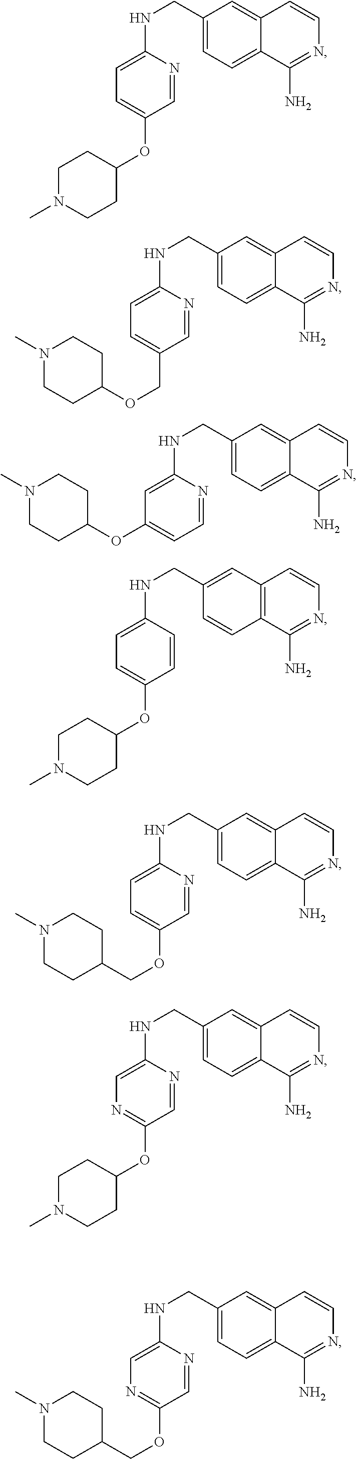

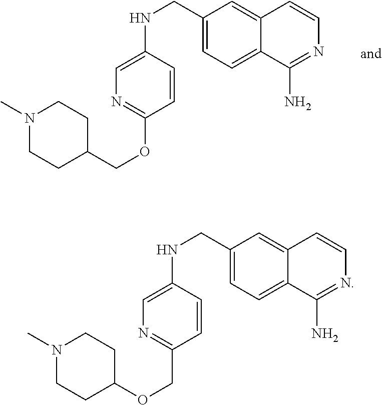 [SMILES: CN1CCC(COc2ccc(NCc3ccc4c(N)nccc4c3)cn2)CC1 ; "and" ; CN1CCC(OCc2ccc(NCc3ccc4c(N)nccc4c3)cn2)CC1]

and pharmaceutically acceptable salts and/or solvates thereof.

The compounds of the invention can be selected from Examples 18.03, 18.04, 18.05, 18.08, 18.1, 18.11, 18.12, 18.15, 18.17, 18.18, 18.202, 18.01, 18.06, 18.07, 18.14, 18.206, 18.209, 18.21, 18.211 and 18.212;

and pharmaceutically acceptable salts and/or solvates thereof.

Preferably, the compounds of the invention can be selected from Examples 18.03, 18.04, 18.05, 18.08, 18.1, 18.11, 18.12, 18.15, 18.17, 18.18 and 18.202; and pharmaceutically acceptable salts and/or solvates thereof.

### Therapeutic Applications

As noted above, the compounds (or pharmaceutically acceptable salts and/or solvates thereof), and pharmaceutical compositions comprising the compounds (or pharmaceutically acceptable salts and/or solvates thereof) of the present invention are inhibitors of FXIIa. They are therefore useful in the treatment of disease conditions for which FXIIa is a causative factor.

Accordingly, the present invention provides a compound of the invention (or a pharmaceutically acceptable salt and/or solvate thereof), or a pharmaceutical composition comprising a compound of the invention (or a pharmaceutically acceptable salt and/or solvate thereof), for use in medicine.

The present invention also provides for the use of a compound of the invention (or a pharmaceutically acceptable salt and/or solvate thereof), or a pharmaceutical composition comprising the compound of the invention (or a pharmaceutically acceptable salt and/or solvate thereof), in the manufacture of a medicament for the treatment or prevention of a disease or condition in which FXIIa activity is implicated.

The present invention also provides a method of treatment of a disease or condition in which FXIIa activity is implicated comprising administration to a subject in need thereof a therapeutically effective amount of a compound of the invention (or a pharmaceutically acceptable salt and/or solvate thereof), or a pharmaceutical composition comprising the compound of the invention (or a pharmaceutically acceptable salt and/or solvate thereof).

As discussed above, FXIIa can mediate the conversion of plasma kallikrein from plasma prekallikrein. Plasma kallikrein can then cause the cleavage of high molecular weight kininogen to generate bradykinin, which is a potent inflammatory hormone. Inhibiting FXIIa has the potential to inhibit (or even prevent) plasma kallikrein production. Thus, the disease or condition in which FXIIa activity is implicated can be a bradykinin-mediated angioedema.

The bradykinin-mediated angioedema can be non-hereditary. For example, the non-hereditary bradykinin-mediated angioedema can be selected from non-hereditary angioedema with normal C1 Inhibitor (AE-nC1 Inh), which can be environmental, hormonal, or drug-induced; acquired angioedema; anaphylaxis associated angioedema; angiotensin converting enzyme (ACE or ace) inhibitor-induced angioedema; dipeptidyl peptidase-4 inhibitor-induced angioedema; and tPA-induced angioedema (tissue plasminogen activator-induced angioedema).

Alternatively, and preferably, the bradykinin-mediated angioedema can be hereditary angioedema (HAE), which is angioedema caused by an inherited dysfunction/fault/mutation. Types of HAE that can be treated with compounds according to the invention include HAE type 1, HAE type 2, and normal C1 inhibitor HAE (normal C1 Inh HAE).

The disease or condition in which FXIIa activity is implicated can be selected from vascular hyperpermeability, stroke including ischemic stroke and haemorrhagic accidents; retinal edema; diabetic retinopathy; DME; retinal vein occlusion; and AMD. These condititions can also be bradykinin-mediated.

As discussed above, FXIIa can activate FXIa to cause a coagulation cascade. Thrombotic disorders are linked to this cascade. Thus, the disease or condition in which FXIIa activity is implicated can be a thrombotic disorder. More specifically, the thrombotic disorder can be thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; and atherosclerosis.

Surfaces of medical devices that come into contact with blood can cause thrombosis. The compounds (or pharmaceutically acceptable salts and/or solvates thereof) and pharmaceutical compositions of the present invention can be coated on the surfaces of devices that come into contact with blood to mitigate the risk of the device causing thrombosis. For instance, they can lower the propensity these devices to clot blood and therefore cause thrombosis. Examples of devices that come into contact with blood include vascular grafts, stents, in dwelling catheters, external catheters, orthopedic prosthesis, cardiac prosthesis, and extracorporeal circulation systems.

Other disease conditions for which FXIIa is a causative factor include: neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

### Combination Therapy

The compounds of the present invention (or pharmaceutically acceptable salts and/or solvates thereof) may be administered in combination with other therapeutic agents. Suitable combination therapies include any compound of the present invention (or a pharmaceutically acceptable salt and/or solvate thereof) combined with one or more agents selected from agents that inhibit platelet-derived growth factor (PDGF), endothelial growth factor (VEGF), integrin alpha5beta1, steroids, other agents that inhibit FXIIa and other inhibitors of inflammation.

Some specific examples of therapeutic agents that may be combined with the compounds of the present invention include those disclosed in EP2281885A and by S. Patel in Retina, 2009 June; 29(6 Suppl):545-8.

Other suitable combination therapies include a compound of the invention (or a pharmaceutically acceptable salt and/or solvate thereof) combined with one or more agents selected from agents that treat HAE (as defined generally herein), for example bradykinin B2 antagonists such icatibant (Firazyr®); plasma kallikrein inhibitors such as ecallantide (Kalbitor®) and lanadelumab (Takhzyro®); or C1 esterase inhibitor such as Cinryze® and Haegarda® and Berinert® and Ruconest®.

Other suitable combination therapies include a compound of the invention (or a pharmaceutically acceptable salt and/or solvate thereof) combined with one or more agents selected from agents that are antithrombotics (as outlined above), for example other Factor XIIa inhibitors, thrombin receptor antagonists, thrombin inhibitors, factor Vila inhibitors, factor Xa inhibitors, factor XIa inhibitors, factor IXa inhibitors, adenosine diphosphate antiplatelet agents (e.g., P2Y12 antagonists), fibrinogen receptor antagonists (e.g. to treat or prevent unstable angina or to prevent reocclusion after angioplasty and restenosis) and aspirin) and platelet aggregation inhibitors.

When combination therapy is employed, the compounds of the present invention and said combination agents may exist in the same or different pharmaceutical compositions, and may be administered separately, sequentially or simultaneously.

The compounds of the present invention can be administered in combination with laser treatment of the retina. The combination of laser therapy with intravitreal injection of an inhibitor of VEGF for the treatment of diabetic macular edema is known (Elman M, Aiello L, Beck R, et al. “Randomized trial evaluating ranibizumab plus prompt or deferred laser or triamcinolone plus prompt laser for diabetic macular edema” Ophthalmology. 27 Apr. 2010).

### Definitions

As noted above, “alkoxy” is a linear O-linked hydrocarbon of between 1 and 3 carbon atoms (C₁-C₃) or a branched O-linked hydrocarbon of between 3 and 4 carbon atoms (C₃-C₄); alkoxy may optionally be substituted with 1 or 2 substituents independently selected from —OH, —CN, —CF₃, —N(R12)₂ and fluoro. Examples of such alkoxy groups include, but are not limited to, C₁ -methoxy, C₂-ethoxy and C₃-n-propoxy for linear alkoxy, and C₃-iso-propoxy, and C₄-sec-butoxy and tert-butoxy for branched alkoxy, optionally substituted as noted aboves. More specifically, alkoxy can be linear groups of between 1 and 3 carbon atoms (C₁-C₃). More specifically, alkoxy can be branched groups of between 3 and 4 carbon atoms (C₃-C₄), optionally substituted as noted above.

As noted above, “alkyl” is a linear saturated hydrocarbon having up to 4 carbon atoms (C₁-C₄) or a branched saturated hydrocarbon of 3 or 4 carbon atoms (C₃-C₄); alkyl may optionally be substituted with 1 or 2 substituents independently selected from (C₁-C₃) alkoxy, —OH, —CN, —NR14R15, —NHCOCH₃, halo, —COOR12, and —CONR14R15. As noted above “alkyl^{b}” is a linear saturated hydrocarbon having up to 4 carbon atoms (C₁-C₄) or a branched saturated hydrocarbon of 3 or 4 carbon atoms (C₃-C₄); alkyl^{b} may optionally be substituted with 1 or 2 substituents independently selected from —OH, —CN, —NHCOCH₃, and halo; Examples of such alkyl or alkyl^{b} groups include, but are not limited, to C₁-methyl, C₂-ethyl, C₃-propyl and C₄-n-butyl, C₃-iso-propyl, C₄-sec-butyl, C₄-iso-butyl and C₄-tert-butyl, optionally substituted as noted above. More specifically, “alkyl” or “alkyl^{b}” can be a linear saturated hydrocarbon having up to 4 carbon atoms (C₁-C₄) or a branched saturated hydrocarbon of between 3 and 4 carbon atoms (C₃-C₄), optionally substituted as noted above.

As noted above, “alkylene” is a bivalent linear saturated hydrocarbon having 1 to 4 carbon atoms (C₁-C₄) or a branched bivalent saturated hydrocarbon having 3 to 4 carbon atoms (C₃-C₄). More specifically, alkylene can be a bivalent linear saturated hydrocarbon having 2 to 4 carbon atoms (C₂-C₄), more specifically having 2 to 3 carbon atoms (C₂-C₃), optionally substituted as noted above.

Halo can be selected from CI, F, Br and I. More specifically, halo can be selected from C1 and F. Preferably, halo is C1.

As noted above “heterocyclyl” is a 4-, 5-, or 6-, membered carbon-containing non-aromatic ring containing one or two ring members that are selected from N, NR16, and O; heterocyclyl may be optionally substituted with 1, 2, 3, or 4 substituents independently selected from alkyl, alkoxy, oxo, —CF₃, —OH, —CN, halo, —COOR12, and —CONR14R15. Heterocyclyl may be a 4-membered carbon-containing non-aromatic ring containing one or two ring members that are selected from N, NR16, and O; heterocyclyl may be optionally substituted with 1, 2, 3, or 4 substituents independently selected from alkyl, alkoxy, oxo, —CF₃, —OH, —CN, halo, —COOR12, and —CONR14R15. Examples of such heterocyclyl groups include azetidinyl and oxetanyl optionally substituted as defined above. Alternatively, heterocyclyl may be a 5-membered carbon-containing non-aromatic ring containing one or two ring members that are selected from N, NR16, and O; heterocyclyl may be optionally substituted with 1, 2, 3, or 4 substituents independently selected from alkyl, alkoxy, oxo, —CF₃, —OH, —CN, halo, —COOR12, and —CONR14R15. Examples of such heterocyclyl groups includes pyrrolidinyl, tetrahydrofuranyl, pyrazolidinyl, imidazolindinyl and 3-dioxolanyl optionally substituted as defined above. Alternatively, heterocyclyl may be a 6-membered carbon-containing non-aromatic ring containing one or two ring members that are selected from N, NR16, and O; heterocyclyl may be optionally substituted with 1, 2, 3, or 4 substituents independently selected from alkyl, alkoxy, oxo, —CF₃, —OH, —CN, halo, —COOR12, and —CONR14R15. Examples of such heterocyclyl groups include piperidinyl, piperazinyl, morpholinyl and 1,4-dioxanyl, optionally substituted as defined above.

The term “O-linked”, such as in “O-linked hydrocarbon residue”, means that the hydrocarbon residue is joined to the remainder of the molecule via an oxygen atom.

The term “N-linked”, such as in “N-linked pyrrolidinyl”, means that the heterocycloalkyl group is joined to the remainder of the molecule via a ring nitrogen atom.

In groups such as —(CH₂)₁₋₃(heterocyclyl), “- ” denotes the point of attachment of the substituent group to the remainder of the molecule.

As is clear from the definitions above, and for the avoidance of any doubt, it will be understood that “Y” is defined above, and does not encompass Yttrium.

“Pharmaceutically acceptable salt” means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming prodrugs are described in ‘The Practice of Medicinal Chemistry, 2n^{d} Ed. pp561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Where compounds of the invention exist in one or more geometrical, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

Unless otherwise stated, the compounds of the invention include compounds that differ only in the presence of one or more isotopically enriched atoms. For example, compounds wherein hydrogen is replaced by deuterium or tritium, or wherein carbon is replaced by ¹³C or ¹⁴C, are within the scope of the present invention. Such compounds are useful, for example, as analytical tools or probes in biological assays.

In the context of the present invention, references herein to “treatment” include references to curative, palliative and prophylactic treatment.

### General Methods

The compounds of the invention may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Compounds of the invention intended for pharmaceutical use may be administered as a solid or liquid, such as a tablet, capsule or solution. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly, the present invention provides a pharmaceutical composition comprising a compound of the invention and a pharmaceutically acceptable carrier, diluent or excipient.

For the treatment of conditions such as retinal vascular permeability associated with diabetic retinopathy and diabetic macular edema, the compounds of the invention may be administered in a form suitable for injection into the ocular region of a patient, in particular, in a form suitable for intra-vitreal injection. It is envisaged that formulations suitable for such use will take the form of sterile solutions of a compound of the invention in a suitable aqueous vehicle. The compositions may be administered to the patient under the supervision of the attending physician.

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, excipients such as sugars (including but not restricted to glucose, manitol, sorbitol, etc.), salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of co-solvents and/or solubility-enhancing agents such as surfactants, micelle structures and cyclodextrins.

The compounds of the invention can be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solids and liquids (including multiple phases or dispersed systems). Exemplary formulations suitable for oral administration include tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Liquid (including multiple phases and dispersed systems) formulations include emulsions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, N.Y., 1980).

For administration to human patients, the total daily dose of the compounds of the invention is typically in the range 0.1 mg and 10,000 mg, or between 1 mg and 5000 mg, or between 10 mg and 1000 mg depending, of course, on the mode of administration.

The total dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

### Synthetic Methods

The compounds of the present invention can be prepared according to the procedures of the following schemes and examples, using appropriate materials, and are further exemplified by the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions, processes and order in which the synthetic steps are performed in the following preparative procedures can be used to prepare these compounds.

The compounds and intermediates of the invention may be isolated in the form of their pharmaceutically acceptable salts, such as those described previously herein above. The interconversion between free form and salt form would be readily known to those skilled in the art.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 4^{th} Edition, 2006, may be used. For example, a common amino protecting group suitable for use herein is tert-butoxy carbonyl (Boc), which is readily removed by treatment with an acid such as trifluoroacetic acid or hydrogen chloride in an organic solvent such as dichloromethane. Alternatively the amino protecting group may be a benzyloxycarbonyl (Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvent. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively a trichloroethyl ester protecting group is removed with zinc in acetic acid. A common hydroxy protecting group suitable for use herein is a methyl ether, deprotection conditions comprise refluxing in 48% aqueous HBr, or by stirring with borane tribromide in an organic solvent such as DCM. Alternatively where a hydroxy group is protected as a benzyl ether, deprotection conditions comprise hydrogenation with a palladium catalyst under a hydrogen atmosphere.

The compounds according to general formula I can be prepared using conventional synthetic methods for example, but not limited to, the routes outlined in Schemes 1-5.

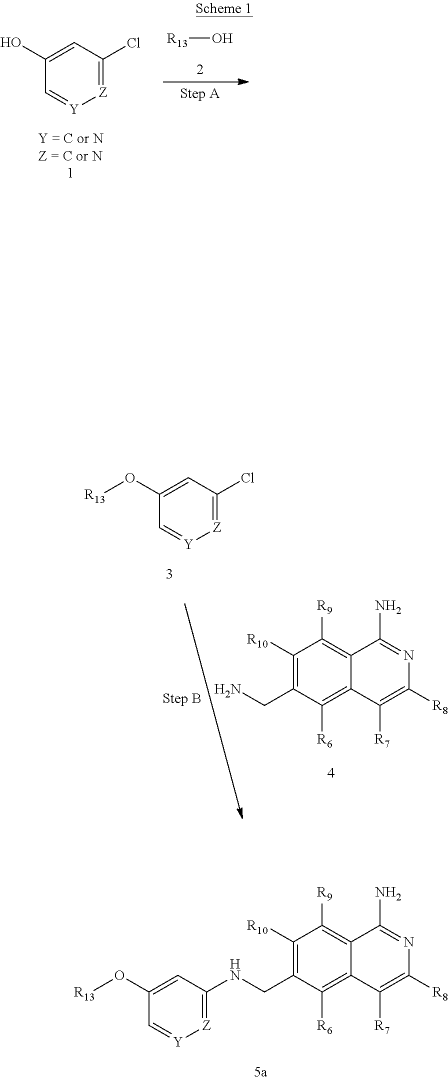

The aryl (or heteroaryl) alcohol 1 is reacted with alcohol 2 under Mitsunobu conditions to give the phenolic ether 3 (Step A). Methods for such transformations are known in the art, for example using DIAD and triphenylphosphine in THF. The chloride, or alternatively bromide, 3 is reacted with amine 4 under Buchwald coupling conditions (Step B). This Buchwald coupling is carried out for example using BrettPhos Pd G3 catalyst in the presence of a base such a NaOtBu or potassium hexamethyldisilazide (KHMDS), in a solvent such as 1,4-dioxane. The amine 4 can be prepared from readily available starting materials using methods known in the art, as described in WO2016083816.

When the oxygen linked substituent is adjacent to a nitrogen in the central ring alternative conditions are possible. For example, as shown in Schemes 2a and 2b standard alkylation reaction via formal deprotonation is a preferred route. Methods for such transformations are known in the art, for example using NaH as a base, alternatively N,N-diisopropylethylamine, potassium carbonate or caesium carbonate; in a solvent such as DMF, dioxane or acetonitrile (Step C).

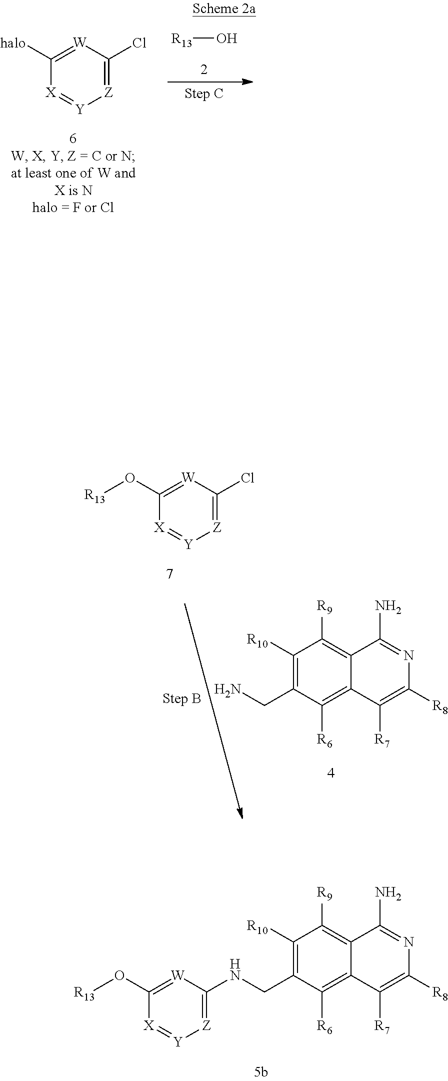

In Scheme 2a, a heteroaryl fluoride, or chloride, 6 is reacted with alcohol 2 in the presence of for example NaH in a solvent such as DMF (Step C). The aforesaid Buchwald coupling (Step B) completes the synthesis.

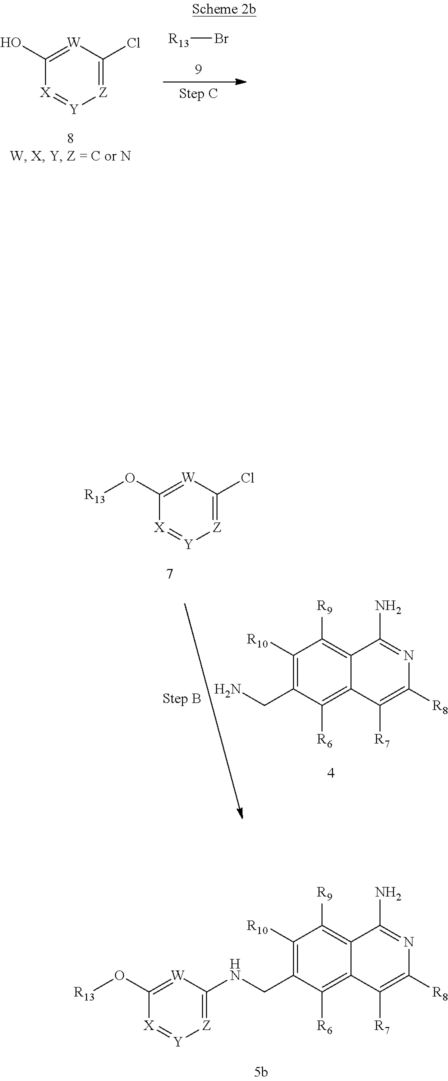

In Scheme 2b, under similar conditions to Scheme 2a, an aryl or heteroaryl alcohol 8 may be reacted with an alkyl bromide 9 (Step C). The aforesaid Buchwald coupling (Step B) completes the synthesis.

In examples where there is a benzylic CH₂, similar conditions to Scheme 2a can be employed as shown in Scheme 3.

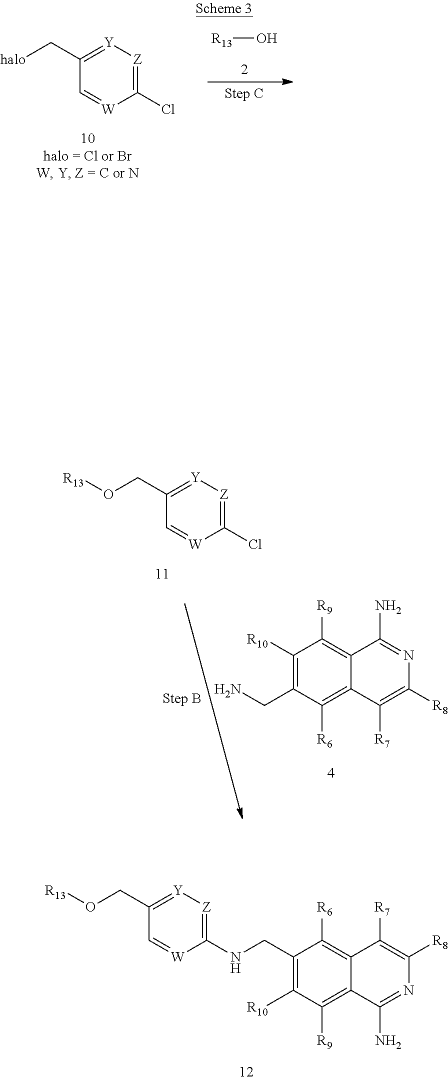

The alkyl halide 10 is reacted with an alcohol 2 using the aforesaid standard alkylation conditions for example in the presence of NaH (Step C). The heteroaryl chloride undergoes the aforesaid Buchwald coupling to complete the synthesis (Step B).

Further modifications can be completed on intermediates as shown in Scheme 4a, or as the final step in Scheme 4b.

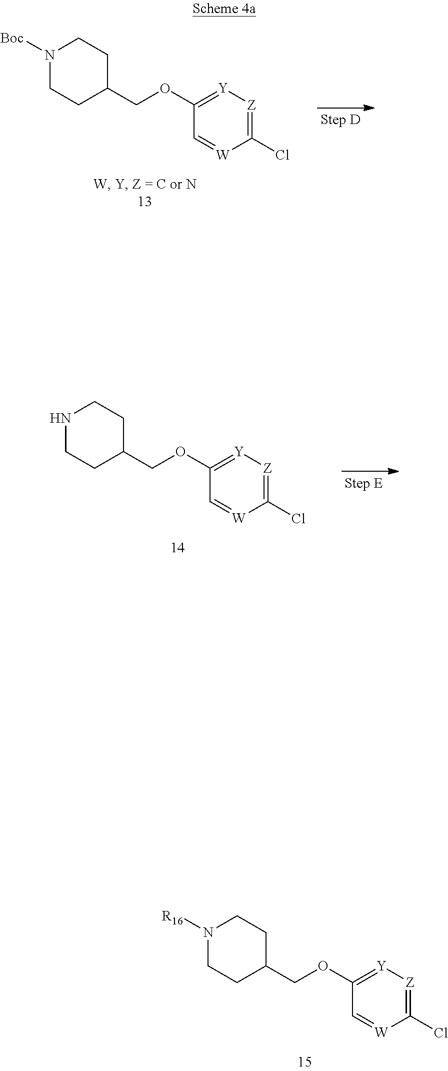

In Scheme 4a the Boc protecting group is removed (Step D) using acidic conditions such as trifluoroacetic acid, or HCI to give amine 14. Typically this intermediate is isolated in the form of the acid salt, for example the trifluoroacetate or HCI. Alkylation of the amine 14 (Step E) may be carried out using standard conditions for such a transformation. For example, amine 14 is treated with formaldehyde (in water) in an appropriate solvent followed by the addition of a reducing agent such as sodium triacetoxyborohydride to give compound 15. Alternative alkylations may be carried out by use of the appropriate alkanone, for example amine 14 is treated with the alkanone, for example acetone, in an organic solvent such as DCM followed by the addition of a reducing agent such as sodium triacetoxyborohydride to give compound 15. Alternative reducing agents include sodium borohydride and sodium cyanoborohydride.

Similar transformations are possible at the final stage of the synthesis as shown in Scheme 4b.

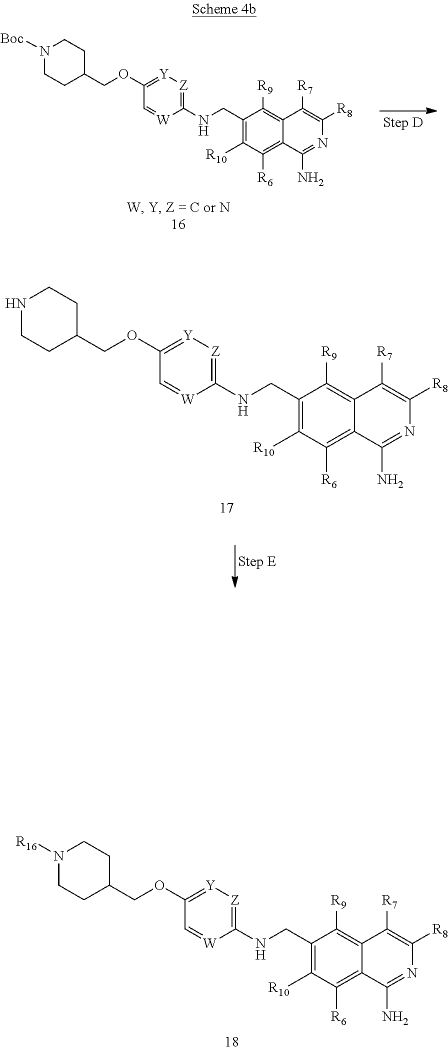

In Scheme 4b the Boc protected amine 16 is deprotected using standard acidic conditions to give amine 17 (Step D), followed by alkylation under the aforesaid conditions described for Scheme 4a (Step E).

When there is an amide group there are alternative synthetic routes available as described in Schemes 5a and 5b.

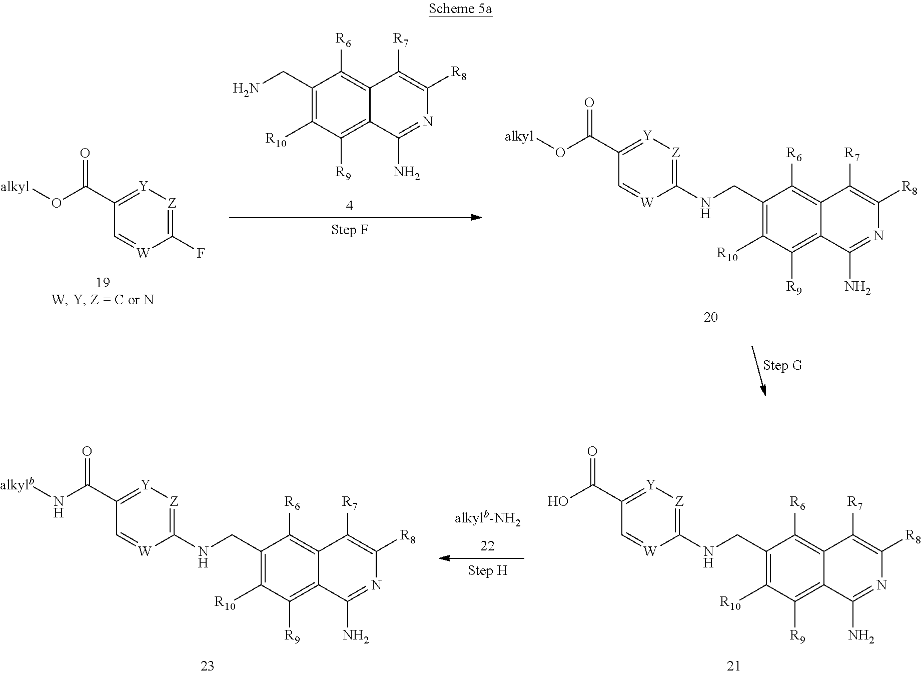

The heteroaryl fluoride 19 is reacted with the amine 4 under standard alkylation conditions for such a transformation (Step F). Typically, heating in the presence of a base, such as potassium carbonate or DIPEA and in a solvent such as DMF, NMP or 1,4-dioxane, both thermally or using microwave irradiation. The ester 20 is hydrolysed (Step G) using standard literature conditions such as NaOH, KOH, LiOH, or TMSOK. The acid (or salt) 21 is coupled to amine (or salt) 22 to give compound 23 (Step H). This coupling is typically carried out using standard coupling conditions such as hydroxybenzotriazole (HOBt) and carbodiimide such as water soluble carbodiimide in the presence of an organic base. Other standard coupling methods include the reaction of acids with amines in the presence of 2-(1H-benzotriazole-1-yl)-1,1,3,3-tetramethylaminium hexafluorophosphate (HBTU) or benzotriazole-1-yl-oxy-tris-pyrrolidino-phosphoium hexafluorophosphate (PyBOP) or bromo-trispyrolidino-phosphonium hexafluorophosphate (PyBroP) or 2-(3H-[1,2,3]triazolo[4,5-b]pyridin-3-yl)-1,1,3,3-tetramethylisouronium hexafluorophosphate(V) (HATU), or 1-ethyl-3-(3-dimethylaminopropyl)carbodiimide (EDC) in the presence of organic bases such as triethylamine, diisopropylethylamine or N-methylmorpholine. Alternatively, the amide formation can take place via an acid chloride in the presence of an organic base. Such acid chlorides can be formed by methods well known in the literature, for example reaction of the acid with oxalyl chloride or thionyl chloride. Alternatively, the carboxylic acid can be activated using 1,1′-carbonyldiimidazole (CDI) and then amine added.

The acid 21 can also be accessed from the nitrile 24 as shown in Scheme 5b.

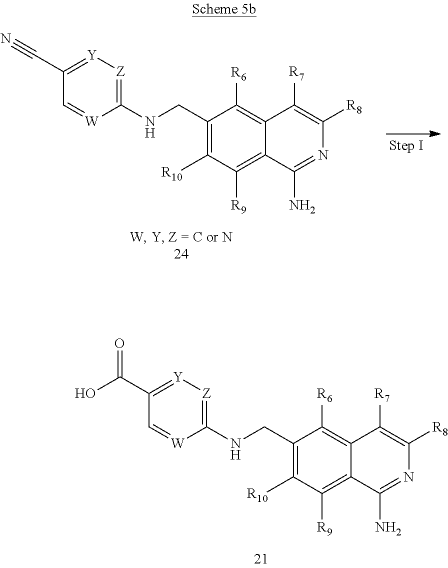

Using standard conditions for such a transformation, nitrile 24 is converted to the acid 21 (Step I). Acid and basic hydrolysis conditions are well known in the literature. Typically the general procedure (Step I) uses a base such as KOH in a solvent such as ethanol.

### EXAMPLES

The invention is illustrated by the following non-limiting examples in which the following abbreviations and definitions are used:

| | |
|---|---|
| Aq | Aqueous solution |
| AIBN | Azobisisobutyronitrile |
| Boc | tert-Butoxy carbonyl |
| BrettPhos Pd G3 | [(2-Di-cyclohexylphosphino-3,6-dimethoxy-2′,4′,6′- |
| | triisopropyl-1,1′-biphenyl)-2-(2′-amino-1,1′-biphenyl)] |
| | palladium(II) methanesulfonate methanesulfonate |
| tBu | Tert-Butyl |
| CDI | 1,1′-Carbonyldiimidazole |
| DCM | Dichloromethane |
| DIAD | Diisopropyl azodicarboxylate |
| DIPEA | N,N-Diisopropylethylamine |
| DMF | N,N-Dimethylformamide |
| DMSO | Dimethyl sulfoxide |
| Eq | Equivalent |
| Et₂O | Diethyl ether |
| Et | Ethyl |
| EtOH | Ethanol |
| EtOAc | Ethyl Acetate |
| HATU | 2-(3H-[l,2,3]triazolo[4,5-b]pyridin-3-yl)-1,1,3,3- |
| | tetramethylisouronium hexafluorophosphate(V) |
| hrs | Hours |
| HOBt | Hydroxybenzotriazole |
| LCMS | Liquid chromatography mass spectrometry |
| Me | Methyl |
| MeCN | Acetonitrile |
| MsCl | Methanesulfonyl chloride |
| MeOH | Methanol |
| Min | Minutes |
| MS | Mass spectrum |
| Ms | Methanesulfonyl |
| NMR | Nuclear magnetic resonance spectrum |
| NMP | N-Methyl-2-pyrrolidone |
| Pet. Ether | Petroleum ether fraction boiling at 60-80° C. |
| Ph | Phenyl |
| iPr | Iso-propyl |
| nPr | n-Propyl |
| SCX | Strong cation exchange |
| SWFI | Sterile water for injection |
| rt | room temperature |
| TBDMS | tert-Butyldimethylsilyl |
| TBME | tert-Butyl methyl ether |
| THF | Tetrahydrofuran |
| TEA | Triethylamine |
| TFA | Trifluoroacetic acid |

All reactions were carried out under an atmosphere of nitrogen unless specified otherwise.

¹H NMR spectra were recorded on a Bruker (500 MHz or 400 MHz) spectrometer and reported as chemical shift (ppm).

Molecular ions were obtained using LCMS with appropriate conditions selected from
Chromolith Speedrod RP-18e column, 50×4.6 mm, with a linear gradient 10% to 90% 0.1% HCO₂H/MeCN into 0.1% HCO₂H/H₂O over 13 min, flow rate 1.5 mL/min;
Agilent, X-Select, acidic, 5-95% MeCN/water over 4 min. Data was collected using a Thermofinnigan Surveyor MSQ mass spectrometer with electrospray ionisation in conjunction with a Thermofinnigan Surveyor LC system;
LCMS (Waters Acquity UPLC, C18, Waters X-Bridge UPLC C18, 1.7 μm, 2.1×30 mm, Basic (0.1% Ammonium Bicarbonate) 3 min method;
LCMS (Agilent, X-Select, Waters X-Select C18, 2.5 μm, 4.6×30 mm, Acidic 4 min method, 95-5 MeCN/water);
LCMS (Agilent, Basic, Waters X-Bridge C18, 2.5 μm, 4.6×30 mm, Basic 4 min method, 5-95 MeCN/water;
Acquity UPLC BEH C18 1.7 μM column, 50×2.1 mm, with a linear gradient 10% to 90% 0.1% HCO2H/MeCN into 0.1% HCO2H/H20 over 3 minutes, flow rate 1 mL/min. Data was collected using a Waters Acquity UPLC mass spectrometer with quadropole dalton, photodiode array and electrospray ionisation detectors.

Flash chromatography was typically carried out over ‘silica’ (silica gel for chromatography, 0.035 to 0.070 mm (220 to 440 mesh) (e.g. Merck silica gel 60)), and an applied pressure of nitrogen up to 10 p.s.i accelerated column elution. Alternatively, pre-prepared cartridges of silica gel were used. Reverse phase preparative HPLC purifications were carried out using a Waters 2525 binary gradient pumping system at flow rates of typically 20 mL/min using a Waters 2996 photodiode array detector.

All solvents and commercial reagents were used as received.

Chemical names were generated using automated software such as ChemDraw (PerkinElmer) or the Autonom software provided as part of the ISIS Draw package from MDL Information Systems or the Chemaxon software provided as a component of MarvinSketch or as a component of the IDBS E-WorkBook.

### Synthesis of Intermediates

### General Method A: Mitsunobu

### 2-Chloro-4-((1-methylpiperidin-4-yl)oxy)pyridine

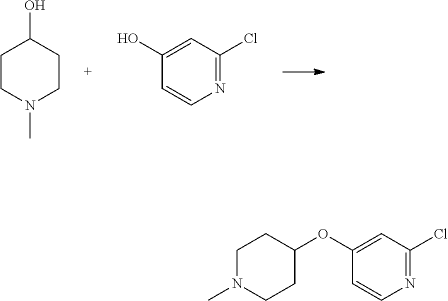

To a solution of 1-methylpiperidin-4-ol (889 mg, 7.72 mmol) in THF (20 mL) at 0° C. was added 2-chloropyridin-4-ol (500 mg, 3.86 mmol) and triphenylphosphine (3.0 g, 11.44 mmol). DIAD (2.3 mL, 11.8 mmol) was then added dropwise over a period of 5 minutes. The solution was allowed to warm to rt and heated to 50° C. for 18 hrs. The reaction was cooled and added directly through SCX and washed with MeOH (20 mL). The required compound was eluted with 7M NH₃ in MeOH (50 mL) and concentrated in vacuo. The crude product was purified by flash chromatography (0-10% MeOH in DCM) to obtain (622 mg, 64% yield) as a clear, colourless oil.

[M+H]⁺=227.1

### General Method B: Buchwald Coupling

### Example 18.03

### 6-(((5-(((1-Methylpiperidin-4-yl)oxy)methyl)pyridin-2-yl)amino)methyl)isoquinolin-1-amine

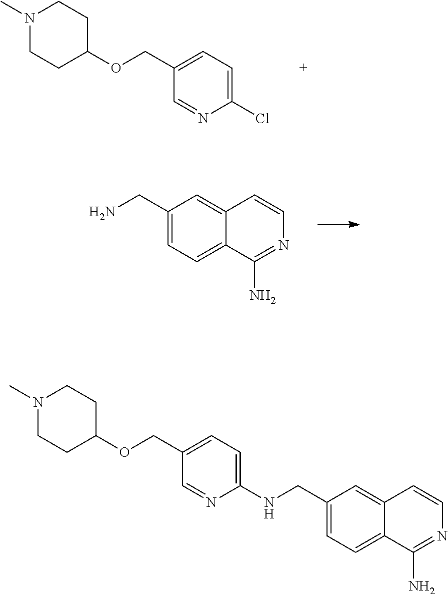 [SMILES: CN1CCC(OCc2ccc(Cl)nc2)CC1 ; NCc1ccc2c(N)nccc2c1 ; CN1CCC(OCc2ccc(NCc3ccc4c(N)nccc4c3)nc2)CC1]

A solution of 2-chloro-5-(((1-methylpiperidin-4-yl)oxy)methyl)pyridine (50 mg, 0.208 mmol), 6-(aminomethyl)isoquinolin-1-amine (36 mg, 0.208 mmol), BrettPhos Pd G3 (19 mg, 0.021 mmol) and sodium tert-butoxide (38 mg, 0.395 mmol) in anhydrous 1,4-dioxane (3 mL) was heated to 120° C. under N2 for 3 hrs. The reaction mixture was diluted with MeOH (5 mL) and added directly through SCX and washed with MeOH (20 mL). The required compound was eluted with 7M NH₃ in MeOH (50 mL) and concentrated in vacuo. The crude product was purified by flash chromatography (0-50% (10% NH₃ in MeOH) in MeCN/EtOAc (50:50)) to obtain the title compound (16 mg, 20% yield) as a yellow solid.

[M+H]⁺=378.4

¹H NMR (500 MHz, DMSO-d6) δ 1.37-1.51 (2H, m), 1.75-1.86 (2H, m), 1.94-2.01 (2H, m), 2.12 (3H, s), 2.54-2.61 (2H, m), 3.27-3.31 (1H, m), 4.27 (2H, s), 4.60 (2H, d, J=6.0 Hz), 6.53 (1H, d, J=8.6 Hz), 6.68 (2H, s), 6.82 (1H, d, J=5.8 Hz), 7.17 (1H, t, J=6.1 Hz), 7.35 (1H, dd, J=8.6, 2.3 Hz), 7.41 (1H, dd, J=8.6, 1.7 Hz), 7.56 (1H, s), 7.74 (1H, d, J=5.7 Hz), 7.89 (1H, d, J=2.3 Hz), 8.11 (1H, d, J=8.6 Hz).

### General Method C: O-Alkylation

### 2-Chloro-6-((1-methylpiperidin-4-yl)oxy)pyridine

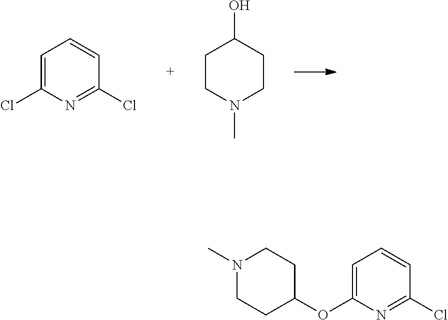

Sodium hydride (60% in mineral oil) (135 mg, 3.38 mmol) was added to a solution of 1-methylpiperidin-4-ol (400 mg, 3.47 mmol) in DMF (3 mL) at 0° C. and stirred for 30 min. The solution was allowed to warm to rt and stirred for 30 min before adding a solution of 2,6-dichloropyridine (500 mg, 3.38 mmol) in DMF (2 mL). The reaction was heated to 80° C. and stirred for 17 hrs. The reaction was cooled and quenched with H₂O (2 mL) before being passed directly through SCX and washed with MeOH (20 mL). The required compound was eluted with 7M NH₃ in MeOH (50 mL) and concentrated in vacuo. The crude product was purified by flash chromatography (0-10% (1% NH₃ in MeOH) in DCM) to obtain the title compound (438 mg, 56% yield) as a white solid.

[M+H]⁺=227.1

¹H NMR (500 MHz, DMSO-d6) δ 1.61-1.71 (2H, m), 1.91-1.99 (2H, m), 2.13-2.20 (5H, m), 2.56-2.67 (2H, m), 4.86-4.97 (1H, m), 6.79 (1H, d, J=7.8 Hz), 7.05 (1H, d, J=7.8, 1.5 Hz), 7.71-7.77 (1H, m).

### General Method D: Boc Deprotection

### 2-Chloro-5-(piperidin-4-ylmethoxy)pyridine

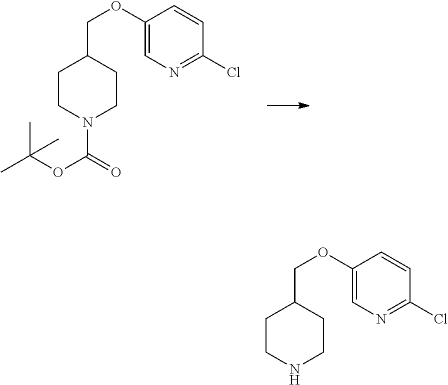 [SMILES: CC(C)(C)OC(=O)N1CCC(COc2ccc(Cl)nc2)CC1 ; Clc1ccc(OCC2CCNCC2)cn1]

TFA (0.7 mL, 9.09 mmol) was added to a solution of tert-butyl 4-(((6-chloropyridin-3-yl)oxy)methyl)piperidine-1-carboxylate (287 mg, 0.88 mmol) in DCM (2 mL) and stirred at rt for 60 min. MeOH (2 mL) was added to the reaction mixture and the solution passed directly through SCX and washed with MeOH (20 mL). The required compound was eluted with 7M NH₃ in MeOH (50 mL) and concentrated in vacuo. The eluted product was concentrated in vacuo to afford the title compound (198 mg, 95% yield) as a white solid.

[M+H]⁺=227.1

¹H NMR (500 MHz, CDCl₃) δ 1.25-1.38 (2H, m), 1.77-1.88 (2H, m), 1.89-2.00 (1H, m), 2.62-2.73 (2H, m), 3.10-3.19 (2H, m), 3.80-3.86 (2H, m), 7.18 (1H, dd), 7.22-7.26 (1H, m), 8.04-8.07 (1H, m).

### General Method E: Reductive Amination

### 2-Chloro-5-((1-methylpiperidin-4-yl)methoxy)pyridine

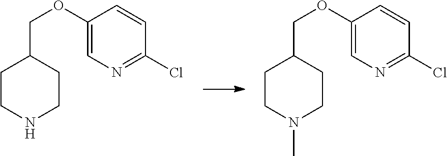 [SMILES: Clc1ccc(OCC2CCNCC2)cn1 ; CN1CCC(COc2ccc(Cl)nc2)CC1]

A solution of 2-chloro-5-(piperidin-4-ylmethoxy)pyridine (198 mg, 0.87 mmol), paraformaldehyde (105 mg, 3.49 mmol) and acetic acid (50 μL, 0.87 mmol) in DCM (4.5 mL) and DMF (0.5 mL) was stirred at rt for 5 min. Sodium triacetoxyborohydride (740 mg, 3.49 mmol) was added and the reaction mixture heated to 40° C. and stirred for 2 hrs. The reaction mixture was cooled and quenched in water (20 mL) and diluted with EtOAc (30 mL) before washing with 1M HCI (aq., 20 mL). The aqueous layer was basified to pH 10 with Na₂CO₃ (sat. aq) and then extracted with DCM (3×50 mL). The organic layer was passed through a phase separator and the resultant filtrate concentrated. The crude product was passed directly through SCX and washed with MeOH (20 mL). The required compound was eluted with 7M NH₃ in MeOH (50 mL) and concentrated in vacuo to afford the title compound (152 mg, 69% yield) as a white solid.

[M+H]⁺=241.1

¹H NMR (500 MHz, DMSO-d6) δ1.22-1.35 (2H, m), 1.64-1.75 (3H, m), 1.80-1.89 (2H, m), 2.15 (3H, s), 2.74-2.80 (2H, m), 3.90 (2H, d, J=6.1 Hz), 7.41 (1H, d, J=8.8 Hz), 7.48 (1H, dd, J=8.8, 3.1 Hz), 8.11 (1H, d, J=3.1 Hz)

### General Method F: SNAr

### Methyl 6-(((1-aminoisoquinolin-6-yl)methyl)amino)nicotinate

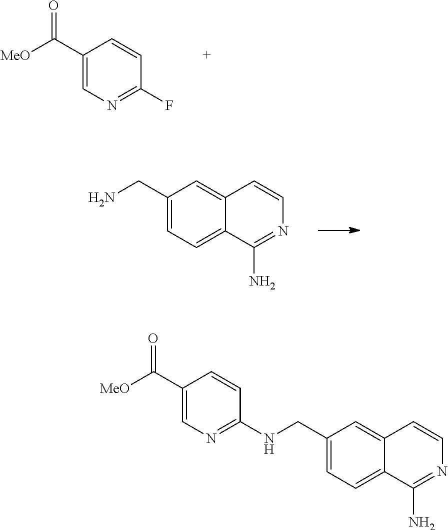 [SMILES: COC(=O)c1ccc(F)nc1 ; NCc1ccc2c(N)nccc2c1 ; COC(=O)c1ccc(NCc2ccc3c(N)nccc3c2)nc1]

To a suspension of 6-(aminomethyl)isoquinolin-1-amine dihydrochloride (349 mg, 1.42 mmol) and potassium carbonate (891 mg, 6.45 mmol) in DMF (5 mL) was added methyl 6-fluoronicotinate (200 mg, 1.29 mmol). The mixture was stirred at 80° C. for 2 hrs then cooled to rt. The reaction was quenched with Na₂CO₃ (sat., aq., 20 mL) and extracted with EtOAc (5×20 mL). The combined organic layers were dried (Na2SO4), filtered and concentrated in vacuo. The crude product was purified by flash chromatography (0-20% (1% NH₃ in MeOH) in DCM) to afford the title compound (364 mg, 83% yield) as a pale white solid.

[M+H]⁺=309.3

¹H NMR (500 MHz, DMSO-d6) δ 3.77 (3H, s), 4.70 (2H, d, J=5.8 Hz), 6.61 (1H, d, J=8.9 Hz), 6.71 (2H, s), 6.84 (1H, d, J=5.8 Hz), 7.41 (1H, dd, J=8.6, 1.8 Hz), 7.57 (1H, d, J=1.7 Hz), 7.76 (1H, d, J=5.8 Hz), 7.85 (1H, dd, J=8.9, 2.3 Hz), 8.03 (1H, t, J=6.0 Hz), 8.13 (1H, d, J=8.6 Hz), 8.57 (1H, d, J=2.3 Hz).

### General Method G: Ester hydrolysis

### Lithium 6-(((1-aminoisoquinolin-6-yl)methyl)amino)nicotinate

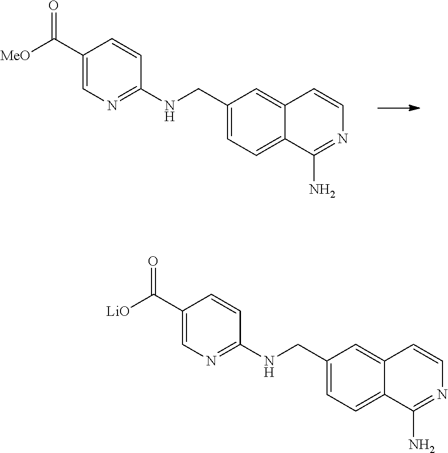

To a suspension of methyl 6-(((1-aminoisoquinolin-6-yl)methyl)amino)nicotinate (50 mg, 0.16 mmol) in a mixture of MeOH (0.2 mL) and THF (1 mL) was added LiOH, 2M in water (0.5 mL, 1.00 mmol). The reaction was stirred at 60° C. for 16 hrs then concentrated in vacuo to give the title compound (49 mg, 97% yield) as an off white solid.

[M+H]⁺=295.2

¹H NMR (500 MHz, DMSO-d6) δ 4.63 (2H, d, J=6.0 Hz), 6.34-6.44 (1H, m), 6.69 (2H, s), 6.83 (1H, d, J=5.8 Hz), 7.16 (1H, t, J=6.1 Hz), 7.42 (1H, dd, J=8.6, 1.7 Hz), 7.57 (1H, d, J=1.6 Hz), 7.74 (1H, d, J=5.8 Hz), 7.81 (1H, dd, J=8.5, 2.2 Hz), 8.12 (1H, d, J=8.6 Hz), 8.45 (1H, d, J=2.2 Hz).

### General Method H: Amide coupling—see Example 18.201

### General Method I: Nitrile Hydrolysis

### 6-(((1-Aminoisoquinolin-6-yl)methyl)amino)picolinic acid

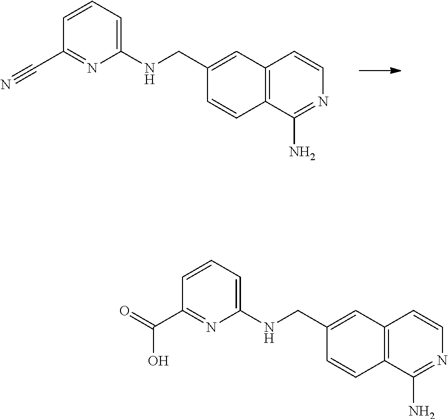 [SMILES: N#Cc1cccc(NCc2ccc3c(N)nccc3c2)n1 ; Nc1nccc2cc(CNc3cccc(C(=O)O)n3)ccc12]

6-(((1-Aminoisoquinolin-6-yl)methyl)amino)picolinonitrile (240 mg, 0.872 mmol) was dissolved in a mixture of ethanol (5 mL) and potassium hydroxide (4 M) (2 mL, 8.00 mmol) and heated in the microwave at 100° C. for 2 hrs. The reaction mixture was concentrated in vacuo to give the title compound (257 mg, 89% yield).

[M+H]⁺=295.1

### Intermediates

### 6-(Aminomethyl)isoquinolin-1-amine

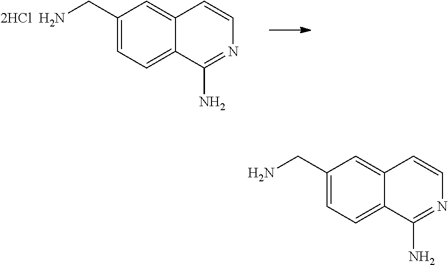 [SMILES: "2HCl" ; NCc1ccc2c(N)nccc2c1 ; NCc1ccc2c(N)nccc2c1]

Sodium carbonate (sat. aq., 40 mL) was added to 6-(aminomethyl)isoquinolin-1-amine dihydrochloride (2.50 g, 10.2 mmol) until pH 10 was achieved. The aqueous suspension was extracted with EtOAc (3×50 mL) and the combined organic layers dried (MgSO₄), filtered and concentrated in vacuo to afford the title compound (1.24 g, 71% yield) as a pale yellow solid.

¹H NMR (500 MHz, DMSO-d6) δ 1.88 (2H, s), 3.84 (2H, s), 6.66 (2H, s), 6.84 (1H, d, J=5.8 Hz), 7.43 (1H, dd, J=8.6, 1.7 Hz), 7.59 (1H, d, J=1.7 Hz), 7.75 (1H, d, J=5.8 Hz), 8.10 (1H, d, J=8.6 Hz).

### 2-Chloro-5-(((1-methylpiperidin-4-yl)oxy)methyl)pyridine

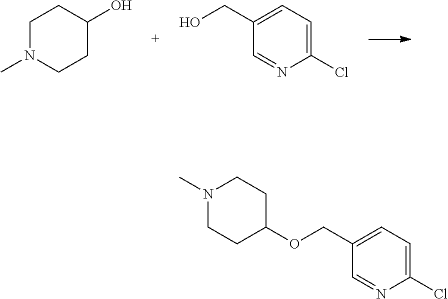

Thionyl chloride (300 μL, 4.11 mmol) was added dropwise to a solution of (6-chloropyridin-3-yl)methanol (500 mg, 3.48 mmol) in DCM (3 mL) at rt and the solution was stirred for 3 hrs. The reaction mixture was concentrated in vacuo and azetroped with MeCN (3×5 mL). This intermediate was reacted with 1-methylpiperidin-4-ol (415 mg, 3.60 mmol) under general method C for 18 hrs at 80° C. The title compound was isolated (166 mg, 19% yield) as a yellow oil.

[M+H]⁺=241.1

¹H NMR (500 MHz, DMSO-d6) δ 1.45-1.56 (2H, m), 1.81-1.92 (2H, m), 1.95-2.06 (2H, m), 2.13 (3H, s), 2.54-2.64 (2H, m), 3.38 (1H, tt, J=8.6, 3.2 Hz), 4.53 (2H, s), 7.50 (1H, d, J=8.2 Hz), 7.81 (1H, dd, J=8.2, 2.5 Hz), 8.37 (1H, d, J=2.5 Hz).

### tert-Butyl 4-(((6-chloropyridin-3-yl)oxy)methyl)piperidine-1-carboxylate

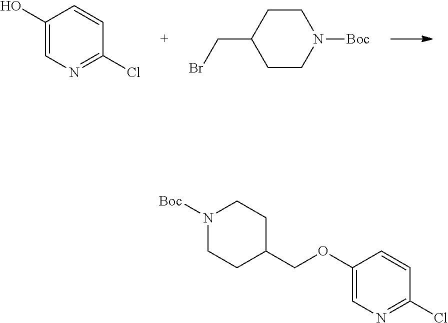 [SMILES: Oc1ccc(Cl)nc1 ; CC(C)(C)OC(=O)N1CCC(CBr)CC1 ; CC(C)(C)OC(=O)N1CCC(COc2ccc(Cl)nc2)CC1]

Following general method C, 6-chloropyridin-3-ol (498 mg, 3.85 mmol) was reacted with tert-butyl 4-(bromomethyl)piperidine-l-carboxylate (1.07 g, 3.85 mmol). The title compound was isolated (364 mg, 28% yield) as an off-white solid.

[M(−tBu 0)+H]⁺=271.0

### Specific Examples of the Present Invention

### Example 18.01

### 6-(((5-((1-Methylpiperidin-4-yl)oxy)pyridin-2-yl)amino)methyl)isoquinolin-1-amine

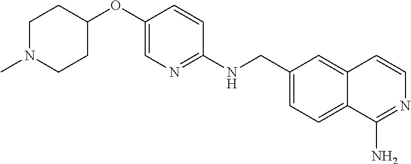 [SMILES: CN1CCC(Oc2ccc(NCc3ccc4c(N)nccc4c3)nc2)CC1]

### 2-Chloro-5-((1-methylpiperidin-4-yl)oxy)pyridine

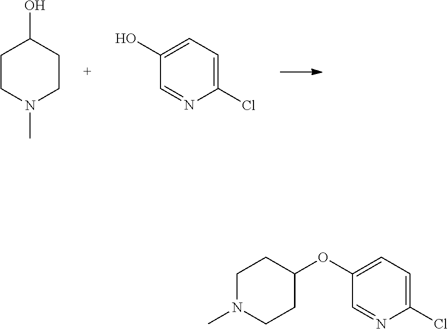

Following general method A, 1-methylpiperidin-4-ol (889 mg, 7.72 mmol) was reacted with 6-chloropyridin-3-ol (500 mg, 3.86 mmol). The title compound was isolated (786 mg, 72% yield) as a clear, colourless oil.

[M+H]⁺=227.1

¹H NMR (500 MHz, DMSO-d6) δ 1.56-1.71 (2H, m), 1.90-1.99 (2H, m), 2.17 (3H, s), 2.57-2.65 (2H, m), 2.72-2.80 (2H, m), 4.41-4.51 (1H, m), 7.41 (1H, d, J=8.7 Hz), 7.52 (1H, dd, J=8.7, 3.2 Hz), 8.12 (1H, d, J=3.2 Hz).

### 6-(((5-((1-Methylpiperidin-4-yl)oxy)pyridin-2-yl)amino)methyl)isoquinolin-1-amine

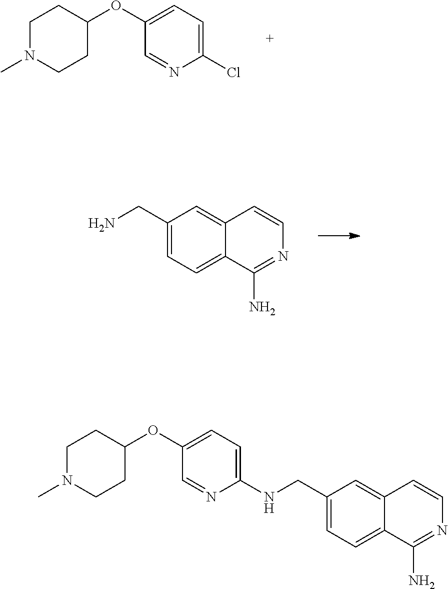 [SMILES: CN1CCC(Oc2ccc(Cl)nc2)CC1 ; NCc1ccc2c(N)nccc2c1 ; CN1CCC(Oc2ccc(NCc3ccc4c(N)nccc4c3)nc2)CC1]

Following general method B, 2-chloro-5-((l-methylpiperidin-4-yl)oxy)pyridine (224 mg, 0.825 mmol) was reacted with 6-(aminomethyl)isoquinolin-1-amine (150 mg, 0.866 mmol). The title compound was isolated (17 mg, 6% yield) as a white solid.

[M+H]⁺=364.1

¹H NMR (DMSO) δ 1.61-1.50 (2H, m), 1.86-1.79 (2H, m), 2.11-2.02 (2H, m), 2.14 (3H, s), 2.60-2.54 (2H, m), 4.08-4.00 (1H, m), 4.55-4.52 (2H, m), 6.52-6.49 (1H, m), 6.68-6.65 (2H, m), 6.81 (2H, t, J=6.1 Hz), 7.16 (1H, dd, J=3.0, 9.0 Hz), 7.41 (1H, dd, J=1.6, 8.6 Hz), 7.56 (1H, s), 7.69 (1H, d, J=2.9 Hz), 7.74 (1H, d, J=5.8 Hz), 8.12-8.08 (1H, m).

### Example 18.02

### 6-[({5-[(1-Methylpiperidin-4-yl)oxy]pyrimidin-2-yl}amino)methyl]isoquinolin-1-amine

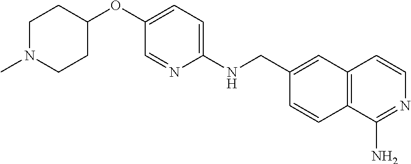 [SMILES: CN1CCC(Oc2ccc(NCc3ccc4c(N)nccc4c3)nc2)CC1]

### 2-Chloro-5-((1-methylpiperidin-4-yl)oxy)pyrimidine

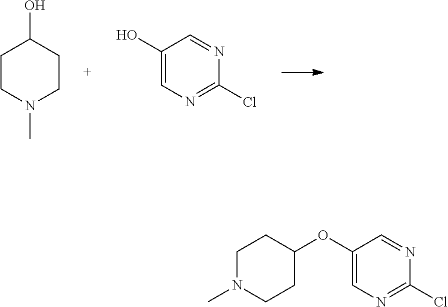

Following general method A, 1-methylpiperidin-4-ol (485 mg, 4.21 mmol) was reacted with 2-chloropyrimidin-5-ol (500 mg, 3.83 mmol). The title compound was isolated (496 mg, 56% yield) as a dark orange oil.

[M+H]⁺=228.1

¹H NMR (500 MHz, DMSO-d6) δ 1.60-1.71 (2H, m), 1.92-2.00 (2H, m), 2.13-2.21 (5H, m), 2.56-2.65 (2H, m), 4.53-4.61 (1H, m), 8.52-8.59 (2H, m).

### 6-[({5-[(1-Methylpiperidin-4-yl)oxy]pyrimidin-2-yl}amino)methyl]isoquinolin-1-amine

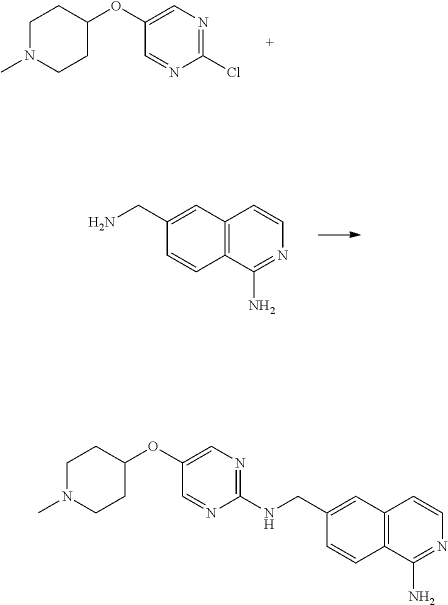 [SMILES: CN1CCC(Oc2cnc(Cl)nc2)CC1 ; NCc1ccc2c(N)nccc2c1 ; CN1CCC(Oc2cnc(NCc3ccc4c(N)nccc4c3)nc2)CC1]

Following general method B, 2-chloro-5-((1-methylpiperidin-4-yl)oxy)pyrimidine (50 mg, 0.220 mmol) was reacted with 6-(aminomethyl)isoquinolin-l-amine (38 mg, 0.219 mmol). The title compound was isolated (15 mg, 18% yield) as a yellow solid.

[M+H]⁺=365.2

¹H NMR (500 MHz, DMSO-d₆) δ 1.57-1.67 (2H, m), 1.81-1.94 (2H, m), 2.14-2.31 (5H, m), 2.60-2.74 (2H, m), 4.06-4.19 (1H, m), 4.56-4.60 (2H, m), 6.67-6.72 (2H, m), 6.81-6.84 (1H, m), 7.38-7.43 (1H, m), 7.50-7.55 (2H, m), 7.73-7.76 (1H, m), 8.08-8.12 (3H, m)

### Example 18.04

### 6-(((5-((1-Methylpiperidin-4-yl)methoxy)pyridin-2-yl)amino)methyl)isoquinolin-1-amine

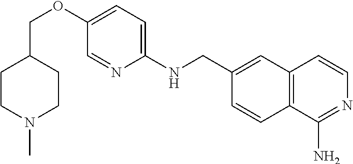 [SMILES: CN1CCC(COc2ccc(NCc3ccc4c(N)nccc4c3)nc2)CC1]

### 6-(((5-((1-Methylpiperidin-4-yl)methoxy)pyridin-2-yl)amino)methyl)isoquinolin-1-amine

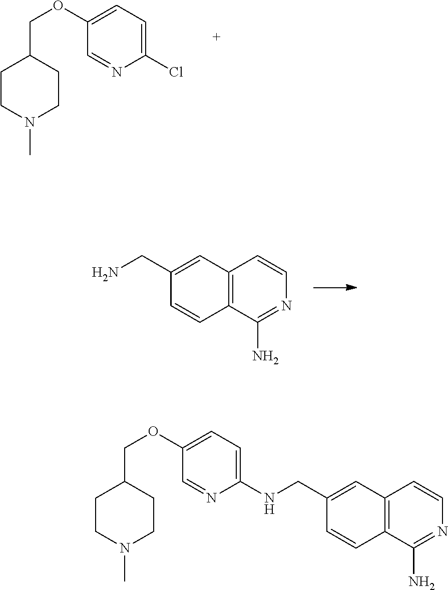 [SMILES: CN1CCC(COc2ccc(Cl)nc2)CC1 ; NCc1ccc2c(N)nccc2c1 ; CN1CCC(COc2ccc(NCc3ccc4c(N)nccc4c3)nc2)CC1]

Following general method B, 2-chloro-5-((1-methylpiperidin-4-yl)methoxy)pyridine (50 mg, 0.21 mmol) was reacted with 6-(aminomethyl)isoquinolin-1-amine (36 mg, 0.21 mmol). The title compound was isolated (28 mg, 35% yield) as a yellow solid.

[M+H]⁺=378.2

¹H NMR (500 MHz, DMSO-d6) δ 1.18-1.32 (2H, m), 1.54-1.65 (1H, m), 1.65-1.73 (2H, m), 1.77-1.87 (2H, m), 2.13 (3H, s), 2.71-2.78 (2H, m), 3.71 (2H, d, J=6.4 Hz), 4.54 (2H, d, J=6.1 Hz), 6.51 (1H, d, J=9.0 Hz), 6.68 (2H, s), 6.77 (1H, t, J=6.1 Hz), 6.82 (1H, d, J=5.8 Hz), 7.13 (1H, dd, J=9.0, 3.0 Hz), 7.41 (1H, dd, J=8.6, 1.7 Hz), 7.56 (1H, s), 7.68 (1H, d, J=3.0 Hz), 7.74 (1H, d, J=5.8 Hz), 8.10 (1H, d, J=8.6 Hz).

### Example 18.08

### 6-(((4-((1-Methylpiperidin-4-yl)oxy)phenyl)amino)methyl)isoquinolin-1-amine

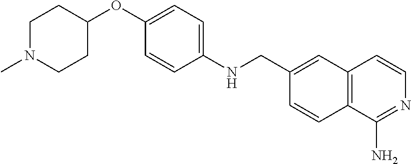 [SMILES: CN1CCC(Oc2ccc(NCc3ccc4c(N)nccc4c3)cc2)CC1]

### 4-(4-Bromophenoxy)piperidine

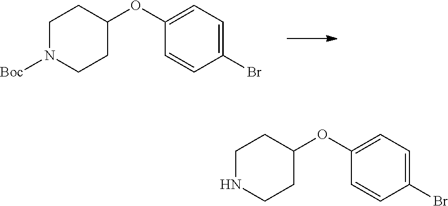 [SMILES: CC(C)(C)OC(=O)N1CCC(Oc2ccc(Br)cc2)CC1 ; Brc1ccc(OC2CCNCC2)cc1]

Following general method D, tert-butyl 4-(4-bromophenoxy)piperidine-l-carboxylate (CAS 769944-78-7, 500 mg, 1.40 mmol) was deprotected using TFA (1.5 mL). The title compound was isolated (352 mg, 98% yield) as a clear, colourless oil.

[M+H]⁺=255.8/257.8

### 4-(4-Bromophenoxy)-1-methylpiperidine

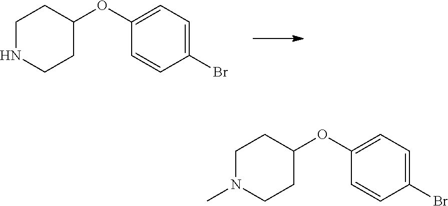

Following general method E, 4-(4-bromophenoxy)piperidine (350 mg, 1.37 mmol) was reacted with paraformaldehyde (160 mg, 5.33 mmol). The title compound was isolated (271 mg, 70% yield) as a white solid.

[M+H]⁺=270.0/272.0

¹H NMR (500 MHz, DMSO-d6) δ 1.75-1.87 (2H, m), 1.95-2.01 (2H, m), 2.23-2.28 (2H, m), 2.30 (3H, s), 2.63-2.72 (2H, m), 4.21 -4.31 (1H, m), 6.76-6.80 (2H, m), 7.33-7.38 (2H, m).

### 6-(((4-((1-Methylpiperidin-4-yl)oxy)phenyl)amino)methyl)isoquinolin-1-amine

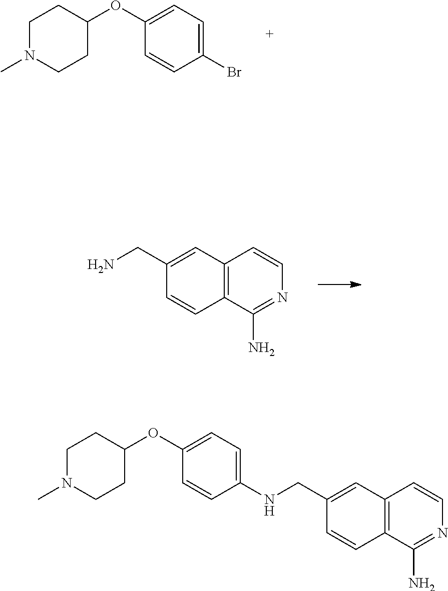 [SMILES: CN1CCC(Oc2ccc(Br)cc2)CC1 ; NCc1ccc2c(N)nccc2c1 ; CN1CCC(Oc2ccc(NCc3ccc4c(N)nccc4c3)cc2)CC1]

Following general method B, 4-(4-bromophenoxy)-1-methylpiperidine (50 mg, 0.19 mmol), was reacted with 6-(aminomethyl)isoquinolin-1-amine (32 mg, 0.19 mmol). The title compound was isolated (20 mg, 28% yield) as an off white solid.

[M+H]⁺=363.2

¹H NMR (500 MHz, DMSO-d6) δ 1.46-1.57 (2H, m), 1.77-1.87 (2H, m), 2.03-2.11 (2H, m), 2.13 (3H, s), 2.54-2.63 (2H, m), 3.97-4.07 (1H, m), 4.30-4.39 (2H, m), 5.94-6.02 (1H, m), 6.49-6.54 (2H, m), 6.64- 6.73 (4H, m), 6.79-6.85 (1H, m), 7.40-7.49 (1H, m), 7.61 (1H, s), 7.71-7.78 (1H, m), 8.12 (1H, dd, J=8.5, 3.7 Hz).

### Example 18.09

### 6-(((5-(2-Morpholinoethom)pyridin-2-yl)amino)methyl)isoquinolin-1-amine

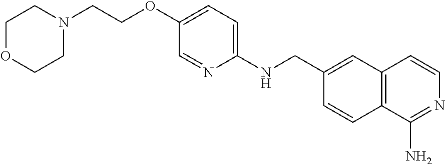 [SMILES: Nc1nccc2cc(CNc3ccc(OCCN4CCOCC4)cn3)ccc12]

### 4-(2-((6-Chloropyridin-3-yl)oxy)ethyl)morpholine

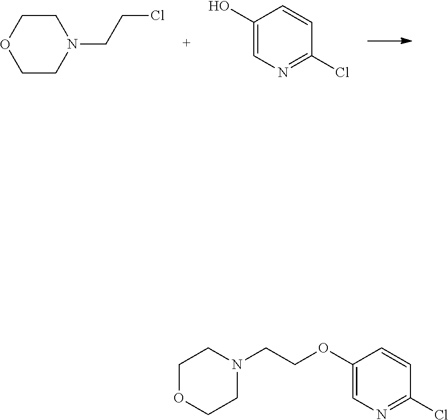 [SMILES: ClCCN1CCOCC1 ; Oc1ccc(Cl)nc1 ; Clc1ccc(OCCN2CCOCC2)cn1]

Following modified general procedure C, 4-(2-chloroethyl)morpholine (462 mg, 3.09 mmol) was reacted with 6-chloropyridin-3-ol (400 mg, 3.09 mmol) and potassium carbonate (640 mg, 4.63 mmol) at 40° C. The title compound (517 mg, 66% yield) was isolated as a clear, pale yellow oil.

[M+H]⁺=243.3

¹H NMR (500 MHz, DMSO-d6) δ 2.46 (4H, t, J=4.7 Hz), 2.69 (2H, t, J=5.7 Hz), 3.57 (4H, t, J=4.7 Hz), 4.17 (2H, t, J=5.7 Hz), 7.42 (1H, d, J=8.7 Hz), 7.50 (1H, dd, J=8.8, 3.2 Hz), 8.13 (1H, d, J=3.1 Hz).

### 6-(((5-(2-Morpholinoethoxy)pyridin-2-yl)amino)methyl)isoquinolin-1-amine

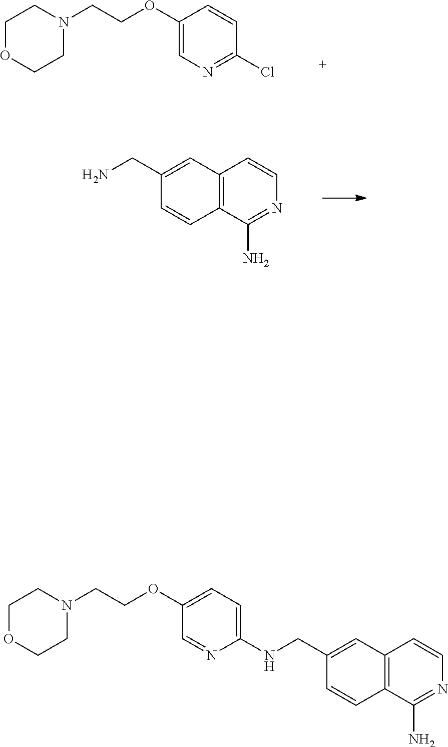 [SMILES: Clc1ccc(OCCN2CCOCC2)cn1 ; NCc1ccc2c(N)nccc2c1 ; Nc1nccc2cc(CNc3ccc(OCCN4CCOCC4)cn3)ccc12]

Following general procedure B, 6-(aminomethyl)isoquinolin-1-amine (40 mg, 0.23 mmol), was reacted with 4-(2-((6-chloropyridin-3-yl)oxy)ethyl)morpholine (50 mg, 0.21 mmol). The title compound was isolated (15 mg, 17% yield) as a pale yellow solid.

[M+H]⁺=380.2

¹H NMR (500 MHz, DMSO-d6) δ 2.41-2.46 (4H, m), 2.62 (2H, t, J=5.8 Hz), 3.52-3.61 (4H, m), 3.98 (2H, t, J=5.8 Hz), 4.54 (2H, d, J=6.1 Hz), 6.51 (1H, d, J=9.0 Hz), 6.67 (2H, s), 6.79 (1H, t, J=6.1 Hz), 6.82 (1H, d, J=5.8 Hz), 7.16 (1H, dd, J=9.0, 3.0 Hz), 7.41 (1H, dd, J=8.6, 1.7 Hz), 7.56 (1H, d, J=1.7 Hz), 7.70 (1H, d, J=3.0 Hz), 7.74 (1H, d, J=5.8 Hz), 8.10 (1H, d, J=8.6 Hz)

### Example 18.10

### 6-(((5-((1-Methylpiperidin-4-yl)methoxy)pyrazin-2-yl)amino)methyl)isoquinolin-1-amine

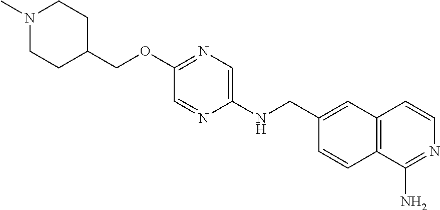 [SMILES: CN1CCC(COc2cnc(NCc3ccc4c(N)nccc4c3)cn2)CC1]

### 2-Chloro-5-((1-methylpiperidin-4-yl)methoxy)pyrazine

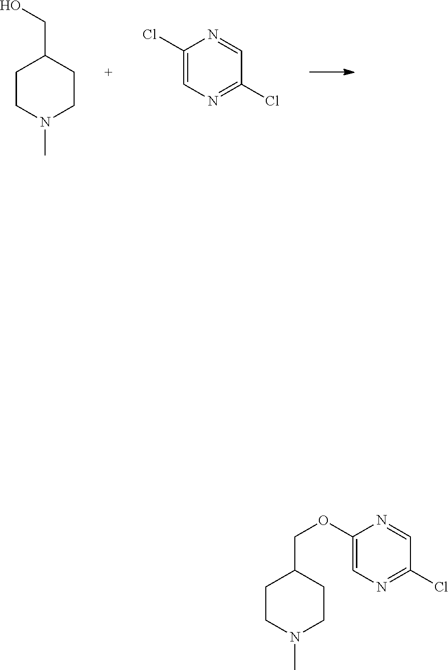

Following general procedure C, (1-methylpiperidin-4-yl)methanol (900 μL, 6.76 mmol) was reacted with 2,5-dichloropyrazine (1.0 g, 6.71 mmol). The title compound (827 mg, 51% yield) was isolated as an off-white solid.

[M+H]⁺=242.0

¹H NMR (500 MHz, DMSO-d6) δ 1.21-1.34 (2H, m,), 1.66-1.75 (3H, m), 1.79-1.87 (2H, m), 2.14 (3H, s), 2.73-2.79 (2H, m), 4.14 (2H, d, J=6.2 Hz), 8.17 (1H, d, J=1.3 Hz), 8.33 (1H, d, J=1.3 Hz).

### 6-(((5-((1-Methylpiperidin-4-yl)methoxy)pyrazin-2-yl)amino)methyl)isoquinolin-1-amine

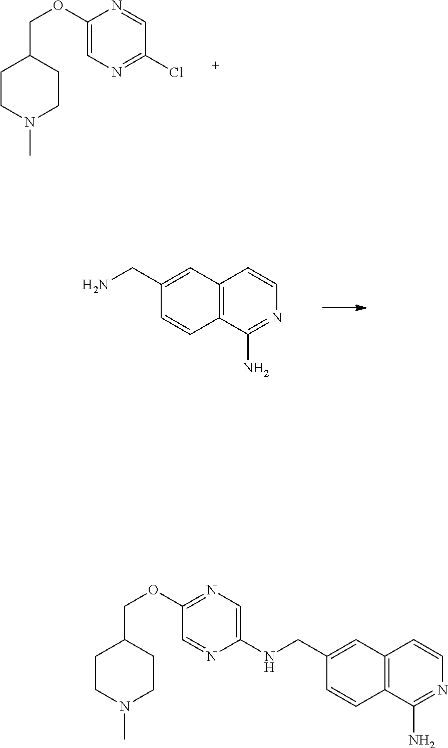 [SMILES: CN1CCC(COc2cnc(Cl)cn2)CC1 ; NCc1ccc2c(N)nccc2c1 ; CN1CCC(COc2cnc(NCc3ccc4c(N)nccc4c3)cn2)CC1]

Following general procedure B, 6-(aminomethyl)isoquinolin-1-amine (0.58 M in 1,4-dioxane) (360 μL, 0.21 mmol) was reacted with 2-chloro-5-((1-methylpiperidin-4-yl)methoxy)pyrazine (50 mg, 0.21 mmol). The title compound (11 mg, 12% yield) was isolated as an orange solid.

[M+H]⁺=379.2

¹H NMR (500 MHz, DMSO-d6) δ 1.18-1.28 (2H, m), 1.57-1.70 (3H, m), 1.78-1.85 (2H, m), 2.13 (3H, s), 2.71-2.77 (2H, m), 3.95 (2H, d, J=6.2 Hz), 4.55 (2H, d, J=6.2 Hz), 6.69 (2H, s), 6.83 (1H, d, J=5.8 Hz), 7.09 (1H, t, J=6.2 Hz), 7.41 (1H, dd, J=8.6, 1.8 Hz), 7.57-7.58 (1H, m), 7.58 (1H, d, J=1.5 Hz), 7.70 (1H, d, J=1.5 Hz), 7.75 (1H, d, J=5.8 Hz), 8.11 (1H, d, J=8.6 Hz).

### Example 18.11

### 6-(((6-((1-Methylpiperidin-4-yl)methoxy)pyridin-3-yl)amino)methyl)isoquinolin-1-amine

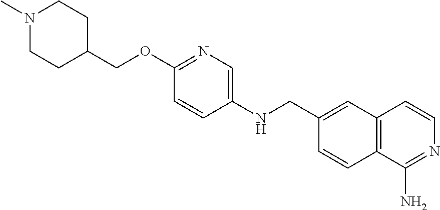 [SMILES: CN1CCC(COc2ccc(NCc3ccc4c(N)nccc4c3)cn2)CC1]

### 5-Bromo-2-((1-methylpiperidin-4-yl)methoxy)pyridine

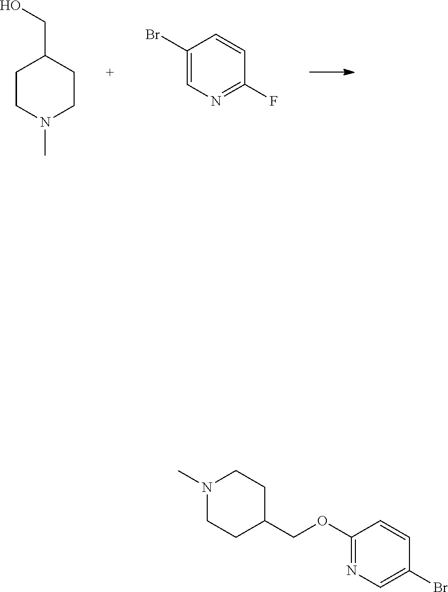

Following general procedure C, 5-bromo-2-fluoropyridine (0.30 mL, 2.91 mmol) was reacted with (1-methylpiperidin-4-yl)methanol (0.40 mL, 3.00 mmol). The title compound (102 mg, 12% yield) was isolated as an off-white solid.

[M+H]⁺=285.0/287.0

¹H NMR (500 MHz, DMSO-d6) δ 1.21-1.34 (2H, m), 1.66-1.73 (3H, m), 1.81-1.93 (2H, m), 2.16 (3H, s), 2.73-2.83 (2H, m), 4.08 (2H, d, J=6.1 Hz), 6.82 (1H, d, J=8.8 Hz), 7.88 (1H, dd, J=8.8, 2.6 Hz), 8.26 (1H, d, J=2.6 Hz).

### 6-(((6-((1-Methylpiperidin-4-yl)methoxy)pyridin-3-yl)amino)methyl)isoquinolin-1-amine

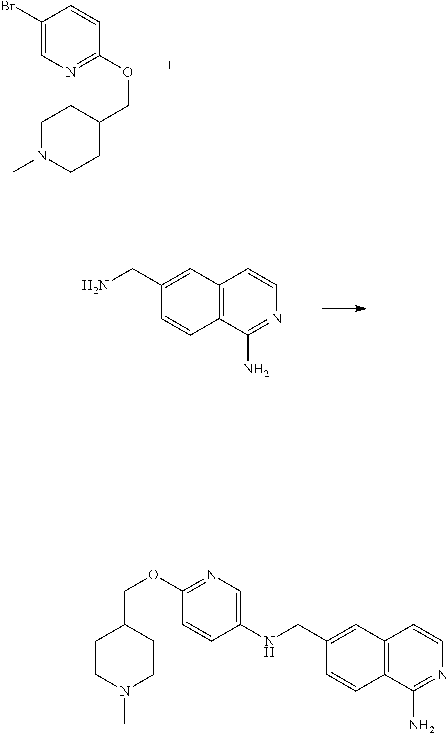 [SMILES: CN1CCC(COc2ccc(Br)cn2)CC1 ; NCc1ccc2c(N)nccc2c1 ; CN1CCC(COc2ccc(NCc3ccc4c(N)nccc4c3)cn2)CC1]

Following general procedure B, 6-(aminomethyl)isoquinolin-1-amine (0.58 M in 1,4-dioxane) (300 μL, 0.17 mmol) was reacted with 5-bromo-2-((1-methylpiperidin-4-yl)methoxy)pyridine (50 mg, 0.18 mmol). The title compound (6 mg, 7% yield) was isolated as a yellow solid.

[M+H]⁺=378.1

¹H NMR (500 MHz, DMSO-d6) δ 1.39-1.51 (2H, m), 1.77-1.82 (2H, m), 1.92-2.00 (2H, m), 2.11 (3H, s), 2.54-2.59 (3H, m), 4.34 (2H, s), 4.43 (2H, d, J=6.1 Hz), 6.59 (1H, t, J=6.2 Hz), 6.70 (2H, s), 6.83 (1H, d, J=5.8 Hz), 6.91 (1H, dd, J=8.5, 2.9 Hz), 7.08 (1H, d, J=8.5 Hz), 7.44 (1H, dd, J=8.5, 1.7), 7.61 (1H, d, J=1.7 Hz), 7.75 (1H, d, J=5.8 Hz), 7.92 (1H, d, J=2.8 Hz), 8.14 (1H, d, J=8.6 Hz).

### Example 18.15

### 5-(((1-Aminoisoquinolin-6-yl)methyl)amino)-2-(1-methylpiperidin-4-yl)methoxy)phenyl)methanol

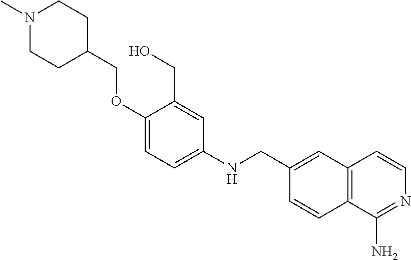 [SMILES: CN1CCC(COc2ccc(NCc3ccc4c(N)nccc4c3)cc2CO)CC1]

### Methyl 5-bromo-2-((1-methylpiperidin-4-yl)methoxy)benzoate

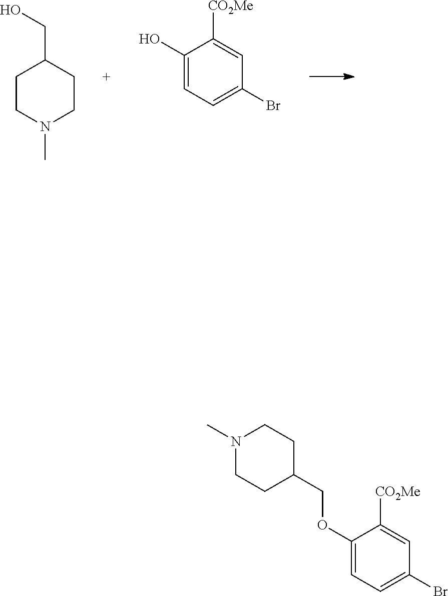

Following general procedure A, methyl 5-bromo-2-hydroxybenzoate (250 mg, 1.08 mmol) was reacted with (1-methylpiperidin-4-yl)methanol (210 mg, 1.62 mmol). The title compound (377 mg, 100% yield) was isolated as a colourless gum.

[M+H]⁺=342.0

### (5-Bromo-2-((1-methylpiperidin-4-yl)methoxy)phenyl)methanol

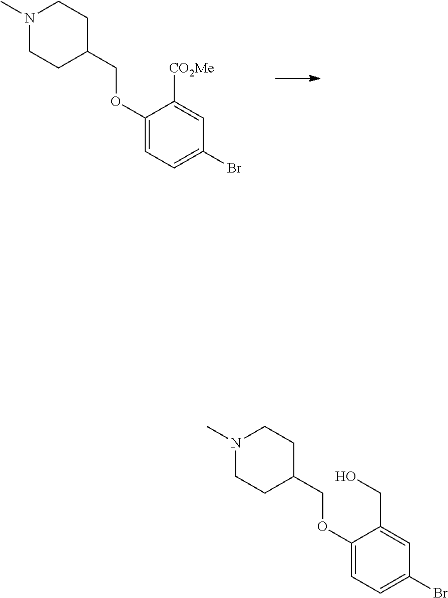 [SMILES: COC(=O)c1cc(Br)ccc1OCC1CCN(C)CC1 ; CN1CCC(COc2ccc(Br)cc2CO)CC1]

To a solution of methyl 5-bromo-2-((1-methylpiperidin-4-yl)methoxy)benzoate (200 mg, 0.58 mmol) in THF (10 mL) at 0° C. was added lithium aluminium hydride (2M in THF) (450 μL, 0.90 mmol) over 20 min then the reaction mixture was stirred for 5 min before being warmed to rt and stirred for 60 min. The reaction was cooled to 0° C. then treated with brine (0.5 mL) before being filtered, dried over MgSO₄, filtered again and concentrated in vacuo. Flash chromatography (0-10% (1% NH₃ in MeOH) in DCM) afforded the title compound (101 mg, 52% yield) as a colourless gum

[M+H]⁺=340.0

¹H NMR (500 MHz, DMSO-d6) δ 1.26-1.35 (2H, m), 1.62-1.73 (3H, m), 1.80-1.88 (2H, m), 2.15 (3H, s), 2.74-2.80 (2H, m), 3.82 (2H, d, J=5.8 Hz), 4.48 (2H, d, J=5.7 Hz), 5.16 (1H, t, J=5.6 Hz), 6.90 (1H, d, J=8.7 Hz), 7.34 (1H, dd, J=8.7, 2.7 Hz), 7.47 (1H, d, J=2.6 Hz).

### (5-(((1-Aminoisoquinolin-6-yl)methyl)amino)-2-((1-methylpiperidin-4-yl)methoxy)phenyl)methanol

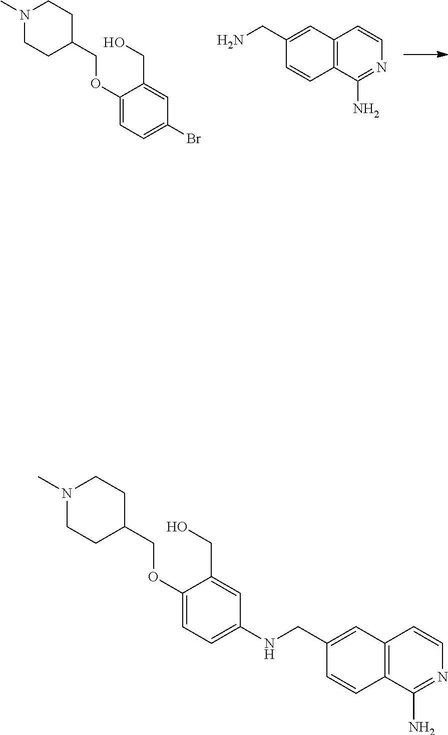 [SMILES: CN1CCC(COc2ccc(Br)cc2CO)CC1 ; NCc1ccc2c(N)nccc2c1 ; CN1CCC(COc2ccc(NCc3ccc4c(N)nccc4c3)cc2CO)CC1]

Following general procedure B, (5-bromo-2-((1-methylpiperidin-4-yl)methoxy)phenyl)methanol (50 mg, 0.16 mmol) was reacted with 6-(aminomethyl)isoquinolin-1-amine. The title compound (5 mg, 7% yield) was isolated as a white solid.

[M+H]⁺=407.2

¹H NMR (500 MHz, MeOD, d4) δ 0.74-0.55 (2H, m), 1.16-0.91 (3H, m), 1.35-1.20 (2H, m), 1.49 (3H, s), 2.16-2.06 (2H, m), 2.94 (2H, d, J=6.1 Hz), 3.65 (2H, s), 3.77 (2H, s), 5.74 (1H, dd, J=8.7, 3.0 Hz), 5.91 (1H, d, J=8.7 Hz), 6.01 (1H, d, J=2.9 Hz), 6.12 (1H, d, J=6.0 Hz), 6.76-6.72 (1H, m), 6.91-6.86 (2H, m), 7.24 (1H, d, J=8.6 Hz)

### Example 18.18

### 6-(((4-((1-Methylpiperidin-4-yl)methoxy)phenyl)amino)methyl)isoquinolin-1-amine

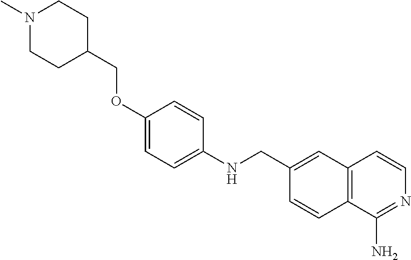 [SMILES: CN1CCC(COc2ccc(NCc3ccc4c(N)nccc4c3)cc2)CC1]

### 4-((4-Bromophenoxy)methyl)-1-methylpiperidine

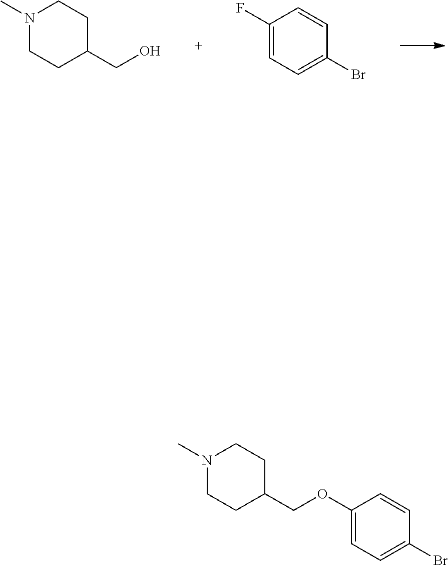

Following general method C, (1-methylpiperidin-4-yl)methanol (206 μL, 1.55 mmol), was reacted with 1-bromo-4-fluorobenzene (204 μL, 1.86 mmol). The title compound was isolated (280 mg, 64% yield) as a colourless solid.

[M+H]⁺=384.2/386.2

¹H NMR (500 MHz, DMSO-d6) δ 1.15-1.34 (2H, m), 1.58-1.74 (3H, m), 1.77-1.90 (2H, m), 2.15 (3H, s), 2.69-2.86 (2H, m), 3.80 (2H, d, J=6.3 Hz), 6.83-6.94 (2H, m), 7.36-7.47 (2H, m)

### 6-(((4-((1-Methylpiperidin-4-yl)methoxy)phenyl)amino)methyl)isoquinolin-1-amine

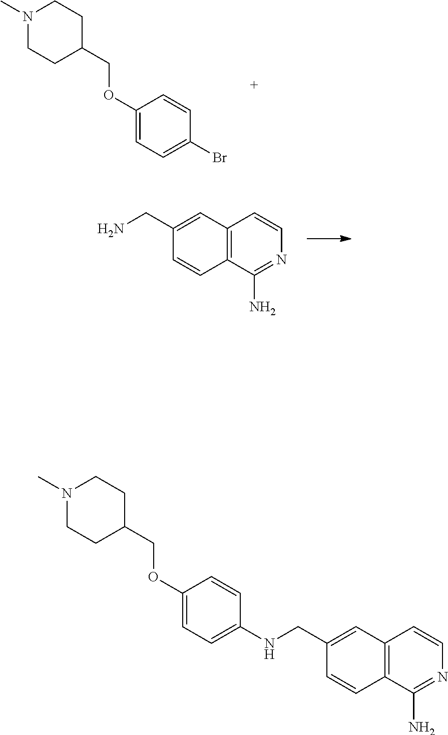 [SMILES: CN1CCC(COc2ccc(Br)cc2)CC1 ; NCc1ccc2c(N)nccc2c1 ; CN1CCC(COc2ccc(NCc3ccc4c(N)nccc4c3)cc2)CC1]

Following general method B, 4-((4-bromophenoxy)methyl)-1-methylpiperidine (50 mg, 0.18 mmol), was reacted with 6-(aminomethyl)isoquinolin-1-amine (34 mg, 0.20 mmol). The title compound was isolated (6 mg, 8% yield) as a colourless solid.

[M+H]⁺=377.2

¹H NMR (500 MHz, DMSO-d6) δ 1.14-1.29 (2H, m), 1.53-1.63 (1H, m), 1.64-1.73 (2H, m), 1.75-1.89 (2H, m), 2.14 (3H, s), 2.68-2.80 (2H, m), 3.64 (2H, d, J=6.4 Hz), 4.34 (2H, d, J=6.0 Hz), 5.93 (1H, t, J=6.2 Hz), 6.48-6.55 (2H, m), 6.65-6.67 (2H, m), 6.68 (2H, s), 6.82 (1H, d, J=5.8 Hz), 7.44 (1H, dd, J=8.6, 1.7 Hz), 7.60 (1H, d, J=1.7 Hz), 7.75 (1H, d, J=5.8 Hz), 8.12 (1H, d, J=8.6 Hz).

### Example 18.19

### 6-(((5-((Piperidin-4-yloxy)methyl)pyridin-2-yl)amino)methyl)isoquinolin-1-amine

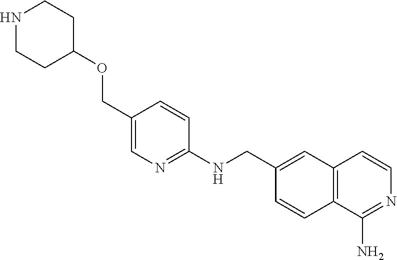 [SMILES: Nc1nccc2cc(CNc3ccc(COC4CCNCC4)cn3)ccc12]

### tert-Butyl 4-((6-chloropyridin-3-yl)methoxy)piperidine-1-carboxylate

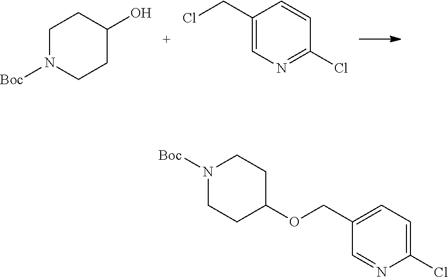

Following general method C, tert-butyl 4-hydroxypiperidine-1-carboxylate (650 mg, 3.23 mmol) was reacted with 2-chloro-5-(chloromethyl)pyridine (500 mg, 3.09 mmol). The title compound was isolated (755 mg, 64% yield) as a dark brown oil.

[M-tBu+H]⁺=270.9

1H NMR (500 MHz, DMSO-d6) δ 1.15-1.28 (2H, m), 1.34-1.46 (9H, m), 1.74-1.88 (2H, m), 2.98-3.12 (2H, m), 3.50-3.72 (3H, m), 4.56 (2H, s), 7.51 (1H, dd, J=8.2, 0.7 Hz), 7.83 (1H, dd, J=8.2, 2.5 Hz), 8.35- 8.40 (1H, m).

### tert-Butyl 4((6-(((1-aminoisoquinolin-6-y)methyl)amino)pyridin-3-yl)methoxy)piperidine-1-carboxylate

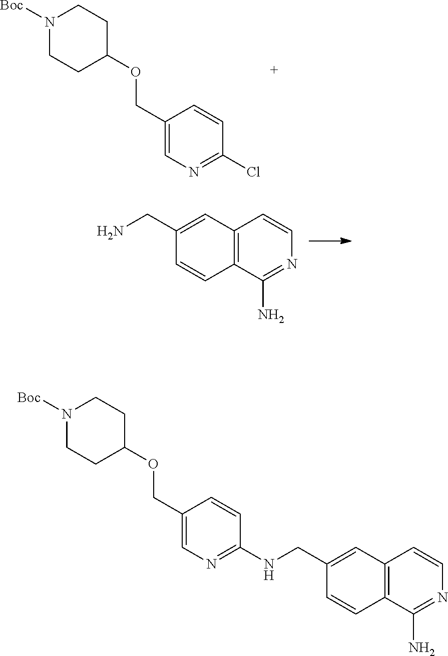

Following general method B, tert-butyl 4-((6-chloropyridin-3-yl)methoxy)piperidine-1-carboxylate (500 mg, 1.53 mmol) was reacted with 6-(aminomethyl)isoquinolin-1-amine (292 mg, 1.68 mmol). The title compound was isolated (274 mg, 36% yield) as an off-white solid.

[M+H]⁺=461.7

¹H NMR (500 MHz, DMSO-d6) δ 1.27-1.37 (2H, m), 1.39 (9H, s), 1.71-1.85 (2H, m), 2.92-3.08 (2H, m), 3.46-3.53 (1H, m), 3.56-3.65 (2H, m), 4.30 (2H, s), 4.61 (2H, d, J=5.8 Hz), 6.54 (1H, d, J=8.6 Hz), 6.68 (2H, s), 6.82 (1H, d, J=5.8 Hz), 7.18 (1H, t, J=6.1 Hz), 7.37 (1H, dd, J=8.5, 2.4 Hz), 7.41 (1H, dd, J=8.6, 1.7 Hz), 7.56 (1H, d, J=1.7 Hz), 7.75 (1H, d, J=5.8 Hz), 7.90 (1H, d, J=2.3 Hz), 8.11 (1H, d, J=8.6 Hz).

### 6-(((5-((Piperidin-4-yloxy)methyl)pyridin-2-yl)amino)methyl)isoquinolin-1-amine

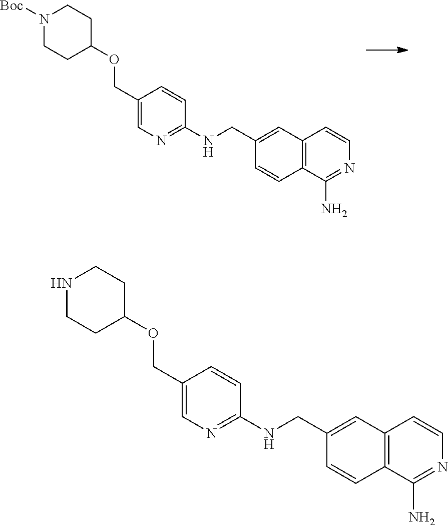 [SMILES: CC(C)(C)OC(=O)N1CCC(OCc2ccc(NCc3ccc4c(N)nccc4c3)nc2)CC1 ; Nc1nccc2cc(CNc3ccc(COC4CCNCC4)cn3)ccc12]

Following general method D, tert-butyl 4-((6-(((1-aminoisoquinolin-6-yl)methyl)amino)pyridin-3-yl)methoxy)piperidine-1-carboxylate (100 mg, 0.216 mmol) was deprotected using TFA (3 mL). The title compound was isolated (82 mg, 99% yield) as a thick brown oil.

[M+H]⁺=364.4

¹H NMR (500 MHz, DMSO-d6) δ 1.28-1.38 (2H, m), 1.79-1.86 (2H, m), 2.51-2.56 (2H, m), 2.88-2.98 (2H, m), 3.35-3.43 (1H, m), 4.29 (2H, s), 4.61 (2H, d, J=5.8 Hz), 6.53 (1H, d, J=8.5 Hz), 6.68 (2H, s), 6.82 (1H, d, J=5.9 Hz), 7.18 (1H, t, J=6.1 Hz), 7.36 (1H, dd, J=8.5, 2.4 Hz), 7.41 (1H, dd, J=8.6, 1.7 Hz), 7.56 (1H, s), 7.75 (1H, d, J=5.8 Hz), 7.89 (1H, d, J=2.3 Hz), 8.11 (1H, d, J=8.6 Hz)

### Example 18.201 and General Method H: Amide Coupling

### 6-(((1-Aminoisoquinolin-6-yl)methyl)amino)-N-((1-methylpiperidin-4-yl)methyl)picolinamide

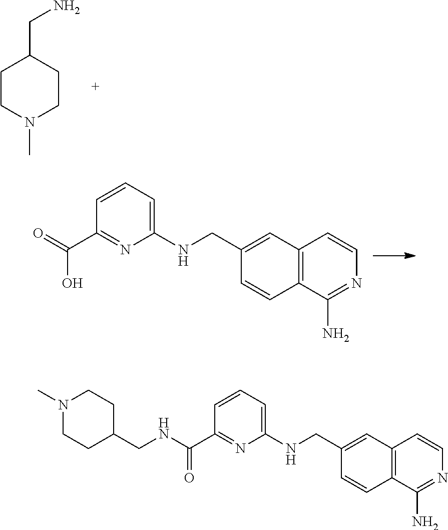

A solution of (1-methylpiperidin-4-yl)methanamine (112 mg, 0.87 mmol), 6-(((1-aminoisoquinolin-6-yl)methyl)amino)picolinic acid (257 mg, 0.87 mmol), DIPEA (0.50 mL, 2.86 mmol) in DMF (5 mL) were stirred at rt for 5 min before adding HATU (516 mg, 1.36 mmol) and stirring the reaction at rt for 4 hrs.

The crude reaction was diluted with MeOH (10 mL) and added directly through SCX and washed with MeOH (50 mL). The required compound was eluted with 7M NH₃ in MeOH (30 mL) and concentrated in vacuo. Flash chromatography (0-20% (1% NH₃ in MeOH) in DCM) afforded the title compound (115 mg, 29% yield) as a yellow solid.

[M+H]⁺=405.2

¹H NMR (500 MHz, DMSO-d6) δ 1.02-1.10 (2H, m), 1.22-1.36 (1H, m), 1.37-1.45 (2H, m), 1.66-1.78 (2H, m), 2.12 (3H, s), 2.61-2.69 (2H, m), 3.05-3.12 (2H, m), 4.65-4.71 (2H, m), 6.69 (2H, s), 6.72-6.78 (1H, m), 6.79-6.84 (1H, m), 7.11-7.18 (1H, m), 7.40-7.48 (1H, m), 7.49-7.58 (2H, m), 7.60-7.64 (1H, m), 7.72-7.77 (1H, m), 8.10-8.15 (2H, m).

### Example 18.202

### 6-(((4-((1-methylpiperidin-4-yl)oxy)pyridin-2-yl)amino)methyl)isoquinolin-1-amine

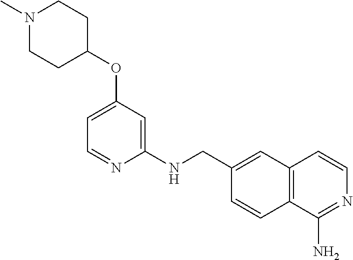 [SMILES: CN1CCC(Oc2ccnc(NCc3ccc4c(N)nccc4c3)c2)CC1]

### 2-Chloro-4-((1-methylpiperidin-4-yl)oxy)pyridine

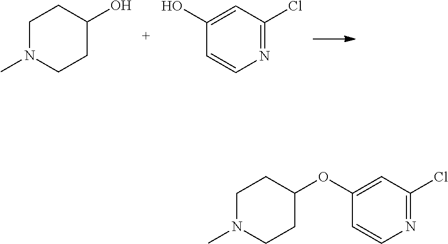

Following general method A, 1-methylpiperidin-4-ol (889 mg, 7.72 mmol) was reacted with 2-chloropyridin-4-ol (500 mg, 3.86 mmol). The title compound was isolated (622 mg, 64% yield) as a clear colourless oil.

[M+H]⁺=227.1

¹H NMR (500 MHz, DMSO-d6) δ 1.10-1.20 (2H, m), 1.59-1.69 (2H, m), 1.90-1.96 (2H, m), 2.18 (3H, s), 2.56-2.61 (2H, m), 4.59 (1H, tt, J=8.4, 4.0 Hz), 7.01 (1H, dd, J=5.8, 2.3 Hz), 7.13 (1H, d, J=2.3 Hz), 8.18 (1H, d, J=5.8 Hz).

### 6-(((4-((1-Methylpiperidin-4-yl)oxy)pyridin-2-yl)amino)methyl)isoquinolin-1-amine

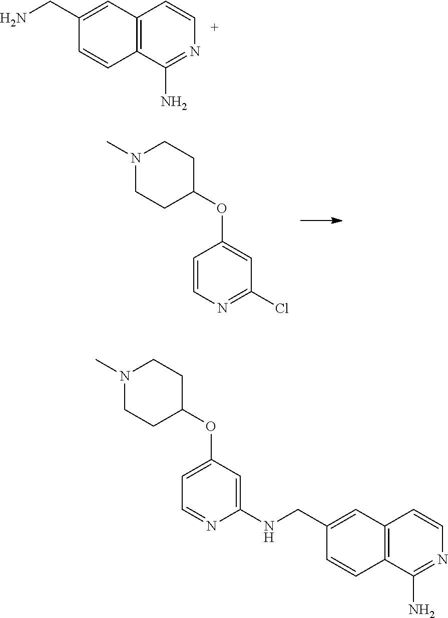

Following general method B, 2-chloro-4-((1-methylpiperidin-4-yl)oxy)pyridine (50 mg, 0.20 mmol) was reacted with 6-(aminomethyl)isoquinolin-1-amine (34 mg, 0.20 mmol). The title compound was isolated (12 mg, 15% yield) as a colourless solid.

[M+H]⁺=364.2

¹H NMR (500 MHz, DMSO-d6) δ 1.58-1.62 (2H, m), 1.82-1.93 (2H, m), 2.14-2.22 (5H, m), 2.57-2.61 (2H, m), 4.26-4.34 (1H, m), 4.55-4.58 (2H, m), 6.00-6.03 (1H, m), 6.13-6.17 (1H, m), 6.67-6.71 (2H, m), 6.82-6.85 (1H, m), 6.97 (1H, t, J=6.2 Hz), 7.39-7.43 (1H, m), 7.55-7.57 (1H, m), 7.73-7.78 (2H, m), 8.10-8.13 (1H, m)

### Example 18.204

### 2-(((1-Aminoisoquinolin-6-yl)methyl)amino)-N-(1-methylpiperidin-4-yl)isonicotinamide

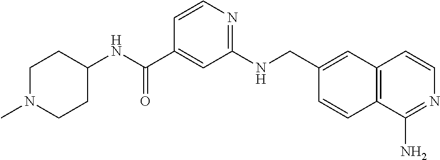 [SMILES: CN1CCC(NC(=O)c2ccnc(NCc3ccc4c(N)nccc4c3)c2)CC1]

### 2-Chloro-N-(1-methylpiperidin-4-yl)isonicotinamide

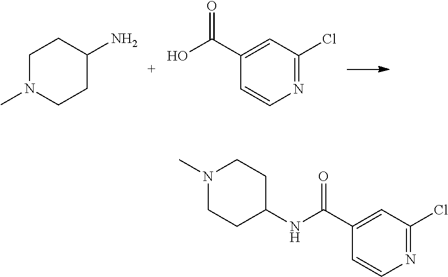

CDI (566 mg, 3.49 mmol) was added to a solution of 2-chloroisonicotinic acid (500 mg, 3.17 mmol) in DMF (20 mL) at rt. The solution was heated to 70° C. for 30 min, before cooling to rt and adding 1-methylpiperidin-4-amine (400 mg, 3.50 mmol) and stirring at rt for 17 hrs. The reaction was passed directly through SCX and washed with MeOH (20 mL). The required compound was eluted with 7M NH₃ in MeOH (50 mL) and concentrated in vacuo. Flash chromatography (0-10% (1% NH₃ in MeOH) in DCM) afforded the title compound (616 mg, 74% yield) as a white solid.

[M+H]⁺=254.0

¹H NMR (500 MHz, DMSO-d6) δ 1.50-1.64 (2H, m), 1.74-1.80 (2H, m), 1.91-1.99 (2H, m), 2.17 (3H, s), 2.74-2.81 (2H, m), 3.64-3.78 (1H, m), 7.76 (1H, dd, J=5.1, 1.5 Hz), 7.86 (1H, s), 8.55 (1H, d, J=5.1 Hz), 8.63 (1H, d, J=7.6 Hz).

### 2-(((1-Aminoisoquinolin-6-yl)methyl)amino)-N-(1-methylpiperidin-4-yl)isonicotinamide

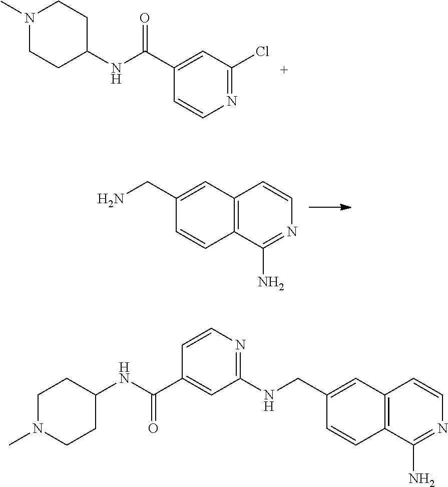

Following general method B, 2-chloro-N-(1-methylpiperidin-4-yl)isonicotinamide (100 mg, 0.39 mmol) was reacted with 6-(aminomethyl)isoquinolin-1-amine (69 mg, 0.40 mmol). The title compound was isolated (75 mg, 41% yield) as a yellow solid.

[M+H]⁺=391.2

¹H NMR (500 MHz, DMSO-d6) δ 1.42-1.64 (2H, m), 1.64-1.78 (2H, m), 1.92 (2H, td, J=11.8, 2.5 Hz), 2.15 (3H, s), 2.72-2.80 (2H, m), 3.61-3.74 (1H, m), 4.65 (2H, d, J=6.0 Hz), 6.68 (2H, s), 6.79-6.86 (2H, m), 6.91 (1H, s), 7.37-7.43 (2H, m), 7.56 (1H, d, J=1.7 Hz), 7.75 (1H, d, J=5.5 Hz), 8.02 (1H, d, J=5.5 Hz), 8.11 (1H, d, J=8.0 Hz), 8.29 (1H, d, J=8.0 Hz).

### Example 18.206

### 6-(((4-(((1-Methylpiperidin-4-yl)oxy)methyl)pyridin-2-yl)amino)methyl)isoquinolin-1-amine

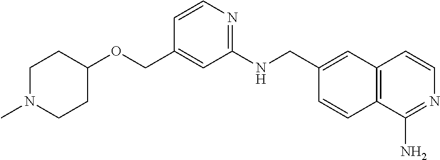 [SMILES: CN1CCC(OCc2ccnc(NCc3ccc4c(N)nccc4c3)c2)CC1]

### 2-Chloro-4-(((1-methylpiperidin-4-yl)oxy)methyl)pyridine

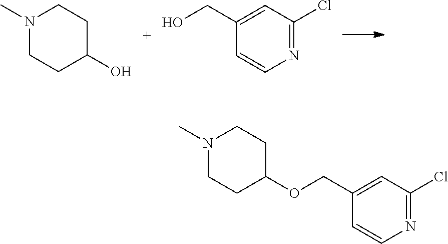

Thionyl chloride (300 μL, 4.11 mmol) was added dropwise to a solution (2-chloropyridin-4-yl)methanol (500 mg, 3.48 mmol) in DCM (3 mL) at rt and the solution was stirred for 3 hrs. The reaction mixture was concentrated in vacuo and azetroped with MeCN (3 ×5 mL). This intermediate was reacted with 1-methylpiperidin-4-ol (415 mg, 3.60 mmol) under general method C for 18 hrs at 80 ° C. Following general procedure C, the title compound was isolated (43 mg, 3% yield) as a brown oil.

[M+H]⁺=241.1

¹H NMR (500 MHz, DMSO-d₆) δ 1.48-1.58 (2H, m), 1.83-1.90 (2H, m), 1.92-2.00 (2H, m), 2.15 (3H, s), 2.56-2.63 (2H, m), 3.35-3.46 (1H, m), 4.58 (2H, s), 7.36 (1H, dd, J=5.0, 1.3 Hz), 7.42 (1H, s), 8.37 (1H, d, J=5.1 Hz)

### 6-(((4-(((1-Methylpiperidin-4-yl)oxy)methyl)pyridin-2-yl)amino)methyl)isoquinolin-1-amine

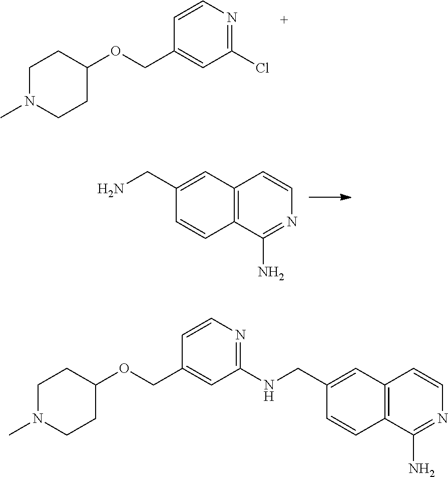 [SMILES: CN1CCC(OCc2ccnc(Cl)c2)CC1 ; "+" ; NCc1ccc2c(N)nccc2c1 ; CN1CCC(OCc2ccnc(NCc3ccc4c(N)nccc4c3)c2)CC1]

Following general method B, 2-chloro-4-(((1-methylpiperidin-4-yl)oxy)methyl)pyridine (43 mg, 0.179 mmol) was reacted with 6-(aminomethyl)isoquinolin-1-amine (31 mg, 0.179 mmol). The title compound was isolated (16 mg, 18% yield) as a yellow glass.

[M+H]⁺=378.2

¹H NMR (500 MHz, DMSO-d6) δ 1.41-1.54 (2H, m), 1.77-1.87 (2H, m), 1.90-2.02 (2H, m), 2.06-2.14 (3H, m), 2.54-2.60 (2H, m), 3.26-3.31 (1H, m), 4.37 (2H, s), 4.60 (2H, d, J=5.9 Hz), 6.41 (1H, dd, J=5.3, 1.4 Hz), 6.51 (1H, s), 6.68 (2H, s), 6.82 (1H, d, J=5.9 Hz), 7.16 (1H, t, J=6.0 Hz), 7.41 (1H, dd, J=8.6, 1.8 Hz), 7.55 (1H, s), 7.75 (1H, d, J=6.0 Hz), 7.88 (1H, d, J=5.2 Hz), 8.11 (1H, d, J=8.6 Hz).

### Example 18.207

### 6-(((6-((1-Methylpiperidin-4-yl)oxy)pyrazin-2-yl)amino)methyl)isoquinolin-1-amine

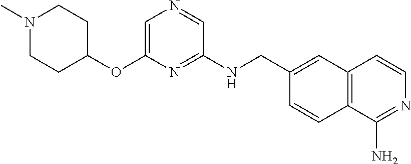 [SMILES: CN1CCC(Oc2cncc(NCc3ccc4c(N)nccc4c3)n2)CC1]

### 2-Chloro-6-((1-methylpiperidin-4-yl)oxy)pyrazine

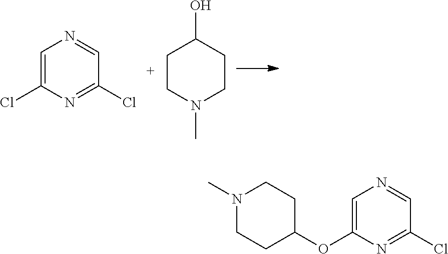

Following general method C, 1-methylpiperidin-4-ol (387 mg, 3.36 mmol) was reacted with 2,6-dichloropyrazine (500 mg, 3.36 mmol). The title compound was isolated (309 mg, 39% yield) as a pale orange oil.

[M+H]⁺=228.1

¹H NMR (500 MHz, DMSO-d6) δ 1.65-1.79 (2H, m), 1.92-2.02 (2H, m), 2.14-2.25 (5H, m), 2.55-2.67 (2H, m), 4.88-5.02 (1H, m), 8.24-8.34 (2H, m).

### 6-(((6-((1-Methylpiperidin-4-yl)oxy)pyrazin-2-yl)amino)methyl)isoquinolin-1-amine

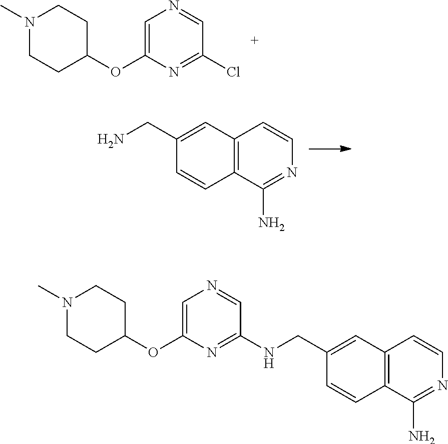

Following general method B, 2-chloro-6-((1-methylpiperidin-4-yl)oxy)pyrazine (60 mg, 0.264 mmol) was reacted with 6-(aminomethyl)isoquinolin-1-amine (46 mg, 0.266 mmol). The title compound was isolated (38 mg, 39% yield) as a yellow solid.

[M+H]+=365.1

¹H NMR (500 MHz, DMSO-d₆) δ 1.42-1.52 (2H, m), 1.67-1.76 (2H, m), 1.89-2.00 (2H, m), 2.12 (3H, s), 2.45-2.50 (2H, m), 4.55 (2H, d, J=5.9 Hz), 4.61-4.69 (1H, m), 6.70 (2H, s), 6.83 (1H, d, J=5.8 Hz), 7.23 (1H, s), 7.40 (1H, dd, J=8.6, 1.7 Hz), 7.53-7.58 (2H, m), 7.72-7.78 (2H, m), 8.12 (1H, d, J=8.5 Hz)

### Example 18.213

### 6-(((4-(Piperidin-4-yloxy)pyridin-2-yl)amino)methyl)isoquinolin-1-amine

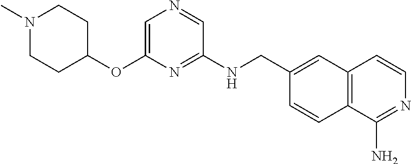 [SMILES: CN1CCC(Oc2cncc(NCc3ccc4c(N)nccc4c3)n2)CC1]

### tert-Butyl 4-((2-chloropyridin-4-yl)oxy)piperidine-1-carboxylate

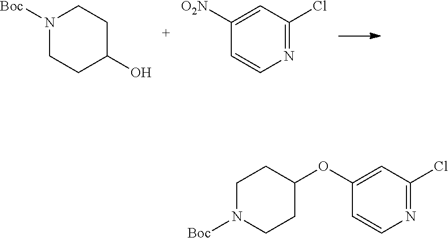

Following general method C, tert-butyl 4-hydroxypiperidine-1-carboxylate (700 mg, 3.48 mmol), was reacted with 2-chloro-4-nitropyridine (500 mg, 3.15 mmol). The title compound was isolated (6 mg, 8% yield) as a pale yellow solid.

[M+H]⁺=313.0

¹H NMR (500 MHz, DMSO-d6) δ 1.41 (9H, s), 1.45-1.58 (2H, m), 1.86-1.98 (2H, m), 3.08-3.22 (2H, m), 3.62-3.73 (2H, m), 4.72-4.83 (1H, m), 7.03 (1H, dd, J=5.8, 2.3 Hz), 7.17 (1H, d, J=2.3 Hz), 8.20 (1H, d, J=5.8 Hz).

### tert-Butyl 4((2-(((1-aminoisoquinolin-6-yl)methyl)amino)pyridin-4-yl)oxy)piperidine-1-carboxylate

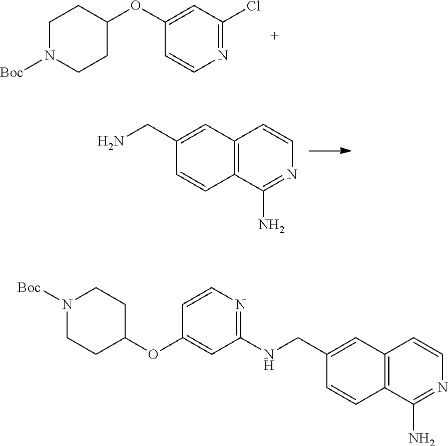

Following general method B, tert-butyl 4-((2-chloropyridin-4-yl)oxy)piperidine-1-carboxylate (500 mg, 1.60 mmol), was reacted with 6-(aminomethyl)isoquinolin-1-amine (305 mg, 1.76 mmol). The title compound was isolated (365 mg, 48% yield) as an off-white solid.

[M+H]⁺=450.5

¹H NMR (500 MHz, DMSO-d6) δ 1.40 (9H, s), 1.43-1.51 (2H, m), 1.78-1.94 (2H, m), 3.07-3.17 (2H, m), 3.58-3.70 (2H, m), 4.47-4.54 (1H, m), 4.57 (2H, d, J=5.9 Hz), 6.03 (1H, d, J=2.2 Hz), 6.18 (1H, dd, J=5.9, 2.2 Hz), 6.69 (2H, s), 6.83 (1H, d, J=5.8 Hz), 7.00 (1H, t, J=6.1 Hz), 7.41 (1H, dd, J=8.6, 1.7 Hz), 7.57 (1H, d, J=1.7 Hz), 7.75 (1H, d, J=5.8 Hz), 7.78 (1H, d, J=5.9 Hz), 8.11 (1H, d, J=8.6 Hz).

### 6-(((4-(Piperidin-4-yloxy)pyridin-2-yl)amino)methyl)isoquinolin-1-amine

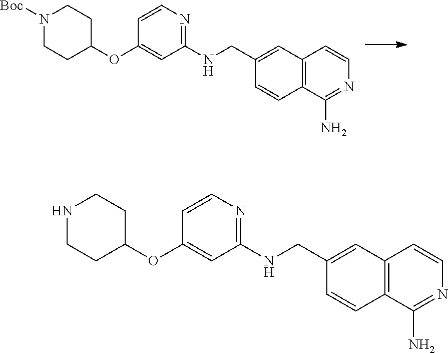 [SMILES: CC(C)(C)OC(=O)N1CCC(Oc2ccnc(NCc3ccc4c(N)nccc4c3)c2)CC1 ; Nc1nccc2cc(CNc3cc(OC4CCNCC4)ccn3)ccc12]

Following general method D, tert-butyl 4-((2-(1-aminoisoquinolin-6-yl)methyl)amino)pyridin-4-yl)oxy)piperidine-1-carboxylate (100 mg, 0.222 mmol) was deprotected using TFA (3 mL). The title compound was isolated (81 mg, 97% yield) as a pale brown solid.

[M+H]⁺=350.1

¹H NMR (500 MHz, DMSO-d6) δ 1.43-1.59 (2H, m), 1.87-1.96 (2H, m), 2.67-2.77 (2H, m), 2.95-3.07 (2H, m), 4.38-4.48 (1H, m), 4.57 (2H, d, J=5.9 Hz), 6.03 (1H, d, J=2.2 Hz), 6.17 (1H, dd, J=5.9, 2.2 Hz), 6.69 (2H, s), 6.83 (1H, d, J=5.8 Hz), 6.99 (1H, t, J=6.2 Hz), 7.41 (1H, dd, J=8.6, 1.7 Hz), 7.57 (1H, d, J=1.7 Hz), 7.75 (1H, d, J=5.8 Hz), 7.78 (1H, d, J=5.9 Hz), 8.11 (1H, d, J=8.6 Hz)

TABLE 3
¹H NMR data of examples (solvent d6 DMSO unless otherwise indicated)

| Ex. No. | NMR write-up |
|---|---|
| 18.01 | 1.61-1.50 (2H, m), 1.86-1.79 (2H, m), 2.11-2.02 (2H, m), 2.14 (3H, s), 2.60-2.54 (2H, m), |
| | 4.08-4.00 (1H, m), 4.55-4.52 (2H, m), 6.52-6.49 (1H, m), 6.68-6.65 (2H, m), 6.81 (2H, t, J = |
| | 6.1 Hz), 7.16 (1H, dd, J = 3.0, 9.0 Hz), 7.41 (1H, dd, J = 1.6, 8.6 Hz), 7.56 (1H, s), 7.69 (1H, d, |
| | J = 2.9 Hz), 7.74 (1H, d, J = 5.8 Hz), 8.12-8.08 (1H, m). |
| 18.02 | 1.57-1.67 (2H, m), 1.81-1.94 (2H, m), 2.14-2.31 (5H, m), 2.60-2.74 (2H, m), 4.06-4.19 |
| | (1H, m), 4.56-4.60 (2H, m), 6.67-6.72 (2H, m), 6.81-6.84 (1H, m), 7.38-7.43 (1H, m), 7.50- |
| | 7.55 (2H, m), 7.73-7.76 (1H, m), 8.08-8.12 (3H, m) |
| 18.03 | 1.37-1.51 (2H, m), 1.75-1.86 (2H, m), 1.94-2.01 (2H, m), 2.12 (3H, s), 2.54-2.61 (2H, m), |
| | 3.27-3.31 (1H, m), 4.27 (2H, s), 4.60 (2H, d, J = 6.0 Hz), 6.53 (1H, d, J = 8.6 Hz), 6.68 (2H, s), |
| | 6.82 (1H, d, J = 5.8 Hz), 7.17 (1H, t, J = 6.1 Hz), 7.35 (1H, dd, J = 8.6, 2.3 Hz), 7.41 (1H, dd, J = |
| | 8.6, 1.7 Hz), 7.56 (1H, s), 7.74 (1H, d, J = 5.7 Hz), 7.89 (1H, d, J = 2.3 Hz), 8.11 (1H, d, J = 8.6 |
| | Hz) |
| 18.04 | 1.18-1.32 (2H, m), 1.54-1.65 (1H, m), 1.65-1.73 (2H, m), 1.77-1.87 (2H, m), 2.13 (3H, s), |
| | 2.71-2.78 (2H, m), 3.71 (2H, d, J = 6.4 Hz), 4.54 (2H, d, J = 6.1 Hz), 6.51 (1H, d, J = 9.0 Hz), |
| | 6.68 (2H, s), 6.77 (1H, t, J = 6.1 Hz), 6.82 (1H, d, J = 5.8 Hz), 7.13 (1H, dd, J = 9.0, 3.0 Hz), 7.41 |
| | (1H, dd, J = 8.6, 1.7 Hz), 7.56 (1H, s), 7.68 (1H, d, J = 3.0 Hz), 7.74 (1H, d, J = 5.8 Hz), 8.10 (1H, |
| | d, J = 8.6 Hz) |
| 18.05 | 1.53-1.66 (2H, m), 1.85-1.92 (2H, m), 2.07-2.14 (2H, m), 2.16 (3H, s), 2.58-2.64 (2H, m), |
| | 4.54 (2H, d, J = 6.1 Hz), 4.63-4.74 (1H, m), 6.69 (2H, s), 6.83 (1H, d, J = 5.8 Hz), 7.10 (1H, t, J = |
| | 6.1 Hz), 7.42 (1H, dd, J = 8.5, 1.8 Hz), 7.57-7.62 (2H, m), 7.67 (1H, d, J = 1.4 Hz), 7.75 (1H, |
| | d, J = 5.8 Hz), 8.12 (1H, d, J = 8.6 Hz) |
| 18.06 | 1.58-1.68 (2H, m), 1.91-2.00 (2H, m), 2.05-2.13 (2H, m), 2.16 (3H, s), 2.58-2.66 (2H, m), |
| | 4.62 (2H, d, J = 5.9 Hz), 4.84-4.94 (1H, m), 6.69 (2H, s), 6.84 (1H, d, J = 5.8 Hz), 6.87 (1H, d, J = |
| | 9.4 Hz), 6.96 (1H, d, J = 9.4 Hz), 7.09 (1H, t, J = 5.9 Hz), 7.43 (1H, dd, J = 8.6, 1.7 Hz), 7.59 |
| | (1H, s), 7.76 (1H, d, J = 5.8 Hz), 8.13 (1H, d, J = 8.6 Hz) |
| 18.07 | (CDCl3) 1.61-1.70 (2H, m), 1.98-2.04 (2H, m), 2.67-2.77 (2H, m), 3.12-3.20 (2H, m), 3.99 |
| | (1H, s), 4.16-4.23 (1H, m), 4.48 (2H, s), 5.12 (2H, s), 6.59-6.64 (2H, m), 6.79-6.83 (2H, m), |
| | 7.04 (1H, d, J = 5.8 Hz), 7.53 (1H, dd, J = 8.6, 1.7 Hz), 7.71 (1H, s), 7.79 (1H, d, J = 8.5 Hz), |
| | 7.96 (1H, d, J = 5.9 Hz) |
| 18.08 | 1.46-1.57 (2H, m), 1.77-1.87 (2H, m), 2.03-2.11 (2H, m), 2.13 (3H, s), 2.54-2.63 (2H, m), |
| | 3.97-4.07 (1H, m), 4.30-4.39 (2H, m), 5.94-6.02 (1H, m), 6.49-6.54 (2H, m), 6.64-6.73 |
| | (4H, m), 6.79-6.85 (1H, m), 7.40-7.49 (1H, m), 7.61 (1H, s), 7.71-7.78 (1H, m), 8.12 (1H, |
| | dd, J = 8.5, 3.7 Hz) |
| 18.09 | 2.41-2.46 (4H, m), 2.62 (2H, t, J = 5.8 Hz), 3.52-3.61 (4H, m), 3.98 (2H, t, J = 5.8 Hz), 4.54 |
| | (2H, d, J = 6.1 Hz), 6.51 (1H, d, J = 9.0 Hz), 6.67 (2H, s), 6.79 (1H, t, J = 6.1 Hz), 6.82 (1H, d, J = |
| | 5.8 Hz), 7.16 (1H, dd, J = 9.0, 3.0 Hz), 7.41 (1H, dd, J = 8.6, 1.7 Hz), 7.56 (1H, d, J = 1.7 Hz), |
| | 7.70 (1H, d, J = 3.0 Hz), 7.74 (1H, d, J = 5.8 Hz), 8.10 (1H, d, J = 8.6 Hz) |
| 18.10 | 1.18-1.28 (2H, m), 1.57-1.70 (3H, m), 1.78-1.85 (2H, m), 2.13 (3H, s), 2.71-2.77 (2H, m), |
| | 3.95 (2H, d, J = 6.2 Hz), 4.55 (2H, d, J = 6.2 Hz), 6.69 (2H, s), 6.83 (1H, d, J = 5.8 Hz), 7.09 (1H, |
| | t, J = 6.2 Hz), 7.41 (1H, dd, J = 8.6, 1.8 Hz), 7.57-7.58 (1H, m), 7.58 (1H, d, J = 1.5 Hz), 7.70 |
| | (1H, d, J = 1.5 Hz), 7.75 (1H, d, J = 5.8 Hz), 8.11 (1H, d, J = 8.6 Hz). |
| 18.11 | 1.39-1.51 (2H, m), 1.77-1.82 (2H, m), 1.92-2.00 (2H, m), 2.11 (3H, s), 2.54-2.59 (3H, m), |
| | 4.34 (2H, s), 4.43 (2H, d, J = 6.1 Hz), 6.59 (1H, t, J = 6.2 Hz), 6.70 (2H, s), 6.83 (1H, d, J = 5.8 |
| | Hz), 6.91 (1H, dd, J = 8.5, 2.9 Hz), 7.08 (1H, d, J = 8.5 Hz), 7.44 (1H, dd, J = 8.5, 1.7), 7.61 (1H, |
| | d, J = 1.7 Hz), 7.75 (1H, d, J = 5.8 Hz), 7.92 (1H, d, J = 2.8 Hz), 8.14 (1H, d, J = 8.6 Hz). |
| 18.12 | 1.39-1.51 (2H, m), 1.77-1.82 (2H, m), 1.92-2.00 (2H, m), 2.11 (3H, s), 2.54-2.59 (3H, m), |
| | 4.34 (2H, s), 4.43 (2H, d, J = 6.1 Hz), 6.59 (1H, t, J = 6.2 Hz), 6.70 (2H, s), 6.83 (1H, d, J = 5.8 |
| | Hz), 6.91 (1H, dd, J = 8.5, 2.9 Hz), 7.08 (1H, d, J = 8.5 Hz), 7.44 (1H, dd, J = 8.5, 1.7), 7.61 (1H, |
| | d, J = 1.7 Hz), 7.75 (1H, d, J = 5.8 Hz), 7.92 (1H, d, J = 2.8 Hz), 8.14 (1H, d, J = 8.6 Hz). |
| 18.14 | 0.94-1.06 (1H, m), 1.42-1.51 (1H, m), 1.57-1.63 (1H, m), 1.64-1.76 (2H, m), 1.81-1.95 |
| | (2H, m), 2.13 (3H, s), 2.56-2.65 (1H, m), 2.76 (1H, d, J = 11.0 Hz), 3.67-3.79 (2H, m), 4.54 |
| | (2H, d, J = 6.1 Hz), 6.51 (1H, d, J = 9.0 Hz), 6.67 (1H, s), 6.78 (1H, t, J = 6.1 Hz), 6.82 (1H, d, J = |
| | 5.8 Hz), 7.14 (1H, dd, J = 9.0, 3.0 Hz), 7.41 (1H, dd, J = 8.6, 1.8 Hz), 7.56 (1H, d, J = 1.7 Hz), |
| | 7.68 (1H, d, J = 3.0 Hz), 7.74 (1H, d, J = 5.8 Hz), 8.10 (1H, d, J = 8.6 Hz). |
| 18.15 | (d4-MeOH) 0.74-0.55 (2H, m), 1.16-0.91 (3H, m), 1.35-1.20 (2H, m), 1.49 (3H, s), 2.16- |
| | 2.06 (2H, m), 2.94 (2H, d, J = 6.1 Hz), 3.65 (2H, s), 3.77 (2H, s), 5.74 (1H, dd, J = 8.7, 3.0 Hz), |
| | 5.91 (1H, d, J = 8.7 Hz), 6.01 (1H, d, J = 2.9 Hz), 6.12 (1H, d, J = 6.0 Hz), 6.76-6.72 (1H, m), |
| | 6.91-6.86 (2H, m), 7.24 (1H, d, J = 8.6 Hz) |
| 18.16 | 1.45-1.59 (2H, m), 1.67-1.77 (2H, m), 1.86-1.98 (2H, m), 2.15 (3H, s), 2.70-2.79 (2H, m), |
| | 3.62-3.72 (1H, m), 4.66 (2H, d, J = 6.0 Hz), 6.54 (1H, d, J = 8.8 Hz), 6.69 (2H, s), 6.83 (1H, d, J = |
| | 5.8 Hz), 7.41 (1H, dd, J = 8.6, 1.7 Hz), 7.55 (1H, s), 7.67 (1H, t, J = 6.1 Hz), 7.75 (1H, d, J = |
| | 5.8 Hz), 7.82 (1H, dd, J = 8.8, 2.4 Hz), 7.88 (1H, d, J = 7.7 Hz), 8.12 (1H, d, J = 8.5 Hz), 8.48 |
| | (1H, d, J = 2.4 Hz). |
| 18.17 | 1.23-1.35 (2H, m), 1.57-1.73 (3H, m), 1.78-1.91 (2H, m), 2.15 (3H, s), 2.60 (2H, t, J = 7.4 |
| | Hz), 2.72-2.81 (2H, m), 3.45-3.51 (2H, m), 3.63 (2H, d, J = 6.1 Hz), 4.32 (2H, d, J = 6.1 Hz), |
| | 4.53 (1H, t, J = 5.3 Hz), 5.84 (1H, t, J = 6.2 Hz), 6.33 (1H, dd, J = 8.7, 2.9 Hz), 6.49 (1H, d, J = |
| | 2.8 Hz), 6.63 (1H, d, J = 8.7 Hz), 6.68 (2H, s), 6.82 (1H, d, J = 5.8 Hz), 7.44 (1H, dd, J = 8.5, 1.8 |
| | Hz), 7.61 (1H, d, J = 1.7 Hz), 7.75 (1H, d, J = 5.8 Hz), 8.11 (1H, d, J = 8.6 Hz) |
| 18.18 | 1.14-1.29 (2H, m), 1.53-1.63 (1H, m), 1.64-1.73 (2H, m), 1.75-1.89 (2H, m), 2.14 (3H, s), |
| | 2.68-2.80 (2H, m), 3.64 (2H, d, J = 6.4 Hz), 4.34 (2H, d, J = 6.0 Hz), 5.93 (1H, t, J = 6.2 Hz), |
| | 6.48-6.55 (2H, m), 6.65-6.67 (2H, m), 6.68 (2H, s), 6.82 (1H, d, J = 5.8 Hz), 7.44 (1H, dd, J = |
| | 8.6, 1.7 Hz), 7.60 (1H, d, J = 1.7 Hz), 7.75 (1H, d, J = 5.8 Hz), 8.12 (1H, d, J = 8.6 Hz). |
| 18.19 | 1.28-1.38 (2H, m), 1.79-1.86 (2H, m), 2.51-2.56 (2H, m), 2.88-2.98 (2H, m), 3.35-3.43 |
| | (1H, m), 4.29 (2H, s), 4.61 (2H, d, J = 5.8 Hz), 6.53 (1H, d, J = 8.5 Hz), 6.68 (2H, s), 6.82 (1H, d, |
| | J = 5.9 Hz), 7.18 (1H, t, J = 6.1 Hz), 7.36 (1H, dd, J = 8.5, 2.4 Hz), 7.41 (1H, dd, J = 8.6, 1.7 Hz), |
| | 7.56 (1H, s), 7.75 (1H, d, J = 5.8 Hz), 7.89 (1H, d, J = 2.3 Hz), 8.11 (1H, d, J = 8.6 Hz) |
| 18.201 | 1.02-1.10 (2H, m),1.22- 1.36 (1H,m),1.37-1.45 (2H, m), 1.66-1.78 (2H, m), 2.12 (3H, s), |
| | 2.61-2.69 (2H, m),3.05- 3.12 (2H,m),4.65-4.71 (2H, m), 6.69 (2H, s), 6.72-6.78 (1H, m), |
| | 6.79-6.84 (1H, m),7.11- 7.18 (1H,m),7.40-7.48(1H, m), 7.49-7.58(2H, m), 7.60-7.64 |
| | (1H, m), 7.72-7.77(1H, m), 8.10-8.15(2H, m) |
| 18.202 | 1.58-1.62 (2H, m), 1.82-1.93 (2H, m), 2.14-2.22 (5H, m), 2.57-2.61 (2H, m), 4.26-4.34 |
| | (1H, m), 4.55-4.58 (2H, m), 6.00-6.03 (1H, m), 6.13-6.17 (1H, m), 6.67-6.71 (2H, m), 6.82- |
| | 6.85 (1H, m), 6.97 (1H, t, J = 6.2 Hz), 7.39-7.43 (1H, m), 7.55-7.57 (1H, m), 7.73-7.78 |
| | (2H, m), 8.10-8.13 (1H, m) |
| 18.203 | 1.37-1.52 (2H, m), 1.68-1.78 (2H, m), 1.90-2.00 (2H, m), 2.13 (3H, s), 3.32 (2H, s), 4.53 |
| | (2H, d, J = 5.9 Hz), 4.61-4.71 (1H, m), 5.80 (1H, d, J = 7.8 Hz), 6.07 (1H, d, J = 7.8 Hz), 6.67 |
| | (2H, s), 6.81 (1H, d, J = 5.9 Hz), 7.20 (1H, t, J = 5.9 Hz), 7.26 (1H, m), 7.40 (1H, dd, J = 8.6, 1.7 |
| | Hz), 7.54 (1H, s), 7.74 (1H, d, J = 5.9 Hz), 8.11 (1H, d, J = 8.6 Hz). |
| 18.204 | 1.42-1.64 (2H, m), 1.64-1.78 (2H, m), 1.92 (2H, td, J = 11.8, 2.5 Hz), 2.15 (3H, s), 2.72-2.80 |
| | (2H, m), 3.61-3.74 (1H, m), 4.65 (2H, d, J = 6.0 Hz), 6.68 (2H, s), 6.79-6.86 (2H, m), 6.91 |
| | (1H, s), 7.37-7.43 (2H, m), 7.56 (1H, d, J = 1.7 Hz), 7.75 (1H, d, J = 5.5 Hz), 8.02 (1H, d, J = |
| | 5.5 Hz), 8.11 (1H, d, J = 8.0 Hz), 8.29 (1H, d, J = 8.0 Hz). |
| 18.205 | 2.09-2.14 (2H, m), 2.15 (3H, s), 2.28-2.35 (2H, m), 3.21 (2H, s), 3.56 (2H, s), 4.63 (2H, d, J = |
| | 6.0 Hz), 6.41-6.47 (2H, m), 6.70 (2H, s), 6.84 (1H, d, J = 5.9 Hz), 7.38 (1H, t, J = 5.9 Hz), 7.42 |
| | (1H, dd, J = 8.6, 1.7 Hz), 7.58 (1H, s), 7.75 (1H, d, J = 5.9 Hz), 8.02 (1H, d), 8.13 (1H, d, J = 8.6 |
| | Hz). |
| 18.206 | 1.41-1.54 (2H, m), 1.77-1.87 (2H, m), 1.90-2.02 (2H, m), 2.06-2.14 (3H, m), 2.54-2.60 |
| | (2H, m), 3.26-3.31 (1H, m), 4.37 (2H, s), 4.60 (2H, d, J = 5.9 Hz), 6.41 (1H, dd, J = 5.3, 1.4 |
| | Hz), 6.51 (1H, s), 6.68 (2H, s), 6.82 (1H, d, J = 5.9 Hz), 7.16 (1H, t, J = 6.0 Hz), 7.41 (1H, dd, J = |
| | 8.6, 1.8 Hz), 7.55 (1H, s), 7.75 (1H, d, J = 6.0 Hz), 7.88 (1H, d, J = 5.2 Hz), 8.11 (1H, d, J = 8.6 |
| | Hz). |
| 18.207 | 1.42-1.52 (2H, m), 1.67-1.76 (2H, m), 1.89-2.00 (2H, m), 2.12 (3H, s), 2.45-2.50 (2H, m), |
| | 4.55 (2H, d, J = 5.9 Hz), 4.61-4.69 (1H, m), 6.70 (2H, s), 6.83 (1H, d, J = 5.8 Hz), 7.23 (1H, s), |
| | 7.40 (1H, dd, J = 8.6, 1.7 Hz), 7.53-7.58 (2H, m), 7.72-7.78 (2H, m), 8.12 (1H, d, J = 8.5 Hz) |
| 18.208 | 2.40-2.45 (4H, m), 2.64 (2H, t, J = 5.7 Hz), 3.51-3.59 (4H, m), 4.04 (2H, t, J = 5.7 Hz), 4.59 |
| | (2H, d, J = 6.1 Hz), 6.03 (1H, d, J = 2.2 Hz), 6.16 (1H, dd, J = 5.9, 2.2 Hz), 6.68 (2H, s), 6.83 (1H, |
| | d, J = 5.8 Hz), 7.01 (1H, t, J = 6.2 Hz), 7.40 (1H, dd, J = 8.6, 1.7 Hz), 7.56 (1H, d, J = 1.7 Hz), |
| | 7.75 (1H, d, J = 5.8 Hz), 7.77 (1H, d, J = 5.9 Hz), 8.11 (1H, d, J = 8.6 Hz, 1H). |
| 18.209 | 1.51-1.64 (2H, m), 1.86-1.93 (2H, m), 2.05-2.12 (2H, m), 2.15 (3H, s), 2.57-2.61 (2H, m), |
| | 4.50-4.68 (2H, m), 4.82-4.93 (1H, m), 5.75 (1H, s), 6.71 (2H, s), 6.84 (1H, d, J = 5.8 Hz), 7.39 |
| | (1H, dd, J = 8.6, 1.7 Hz), 7.55 (1H, s), 7.78-7.74 (2H, m), 8.12-8.16 (2H, m). |
| 18.210 | 1.47-1.59 (1H, m), 1.60-1.70 (2H, m), 1.85-1.99 (1H, m), 2.11-2.20 (1H, m), 2.32 (3H, s), |
| | 2.47-2.51 (1H, m), 2.88-2.99 (1H, m), 3.77 (1H, dd, J = 9.7, 6.0 Hz), 3.92 (1H, dd, J = 9.7, |
| | 5.3 Hz), 4.59 (2H, d, J = 6.1 Hz), 6.03 (1H, d, J = 2.3 Hz), 6.15 (1H, dd, J = 5.8, 2.2 Hz), 6.68 |
| | (2H, s), 6.83 (1H, d, J = 5.7 Hz), 7.01 (1H, t, J = 6.2 Hz), 7.41 (1H, dd, J = 8.6, 1.7 Hz), 7.56 (1H, |
| | d, J = 1.7 Hz), 7.73-7.78 (2H, m), 8.11 (1H, d, J = 8.6 Hz). |
| 18.211 | 1.39-1.59 (2H, m), 1.72-1.87 (2H, m), 1.96-2.08 (2H, m), 2.13 (3H, s), 2.50-2.56 (2H, m), |
| | 4.12 (1H, dd, J = 8.5, 4.5 Hz), 4.38 (2H, d, J = 6.0 Hz), 6.02-6.12 (2H, m), 6.15-6.22 (1H, m), |
| | 6.29-6.39 (1H, m), 6.69 (2H, s), 6.83 (1H, d, J = 5.8 Hz), 6.86-6.96 (1H, m), 7.43 (1H, dd, J = |
| | 8.6, 1.7 Hz), 7.60 (1H, d, J = 1.5 Hz), 7.75 (1H, d, J = 5.8 Hz), 8.13 (1H, d, J = 8.6 Hz) |
| 18.212 | 1.46-1.56 (2H, m), 1.75-1.81 (2H, m), 1.97-2.09 (2H, m), 2.13 (3H, s), 2.52-2.55 (2H, m), |
| | 4.17-4.24 (1H, m), 4.43 (2H, d, J = 6.0 Hz), 6.44 (1H, t, J = 2.4 Hz), 6.63 (1H, t, J = 6.0 Hz), |
| | 6.71 (2H, s), 6.84 (1H, d, J = 5.8 Hz), 7.44 (1H, dd, J = 8.6, 1.7 Hz), 7.46 (1H, d, J = 2.4 Hz), |
| | 7.60-7.65 (2H, m), 7.76 (1H, d, J = 5.8 Hz), 8.14 (1H, d, J = 8.6 Hz). |
| 18.213 | 1.43-1.59 (2H, m), 1.87-1.96 (2H, m), 2.67-2.77 (2H, m), 2.95-3.07 (2H, m), 4.38-4.48 |
| | (1H, m), 4.57 (2H, d, J = 5.9 Hz), 6.03 (1H, d, J = 2.2 Hz), 6.17 (1H, dd, J = 5.9, 2.2 Hz), 6.69 |
| | (2H, s), 6.83 (1H, d, J = 5.8 Hz), 6.99 (1H, t, J = 6.2 Hz), 7.41 (1H, dd, J = 8.6, 1.7 Hz), 7.57 (1H, |
| | d, J = 1.7 Hz), 7.75 (1H, d, J = 5.8 Hz), 7.78 (1H, d, J = 5.9 Hz), 8.11 (1H, d, J = 8.6 Hz). |

### Biological Methods

### Determination of the % Inhibition for FXIIa

Factor XIIa inhibitory activity in vitro was determined using standard published methods (see e.g. Shori et al., Biochem. Pharmacol., 1992,43, 1209; Baeriswyl et al., ACS Chem. Biol., 2015, 10 (8) 1861; Bouckaert et al., European Journal of Medicinal Chemistry 110 (2016) 181). Human Factor XIIa (Enzyme Research Laboratories) was incubated at 25° C. with the fluorogenic substrate H-DPro-Phe-Arg-AFC and various concentrations of the test compound. Residual enzyme activity (initial rate of reaction) was determined by measuring the change in optical absorbance at 410 nm and the IC50 value for the test compound was determined.

Data acquired from this assay are shown in Table 2 below using the following scale:

| Category | IC₅₀ (nM) |
|---|---|
| A | <1,000 |
| B | 1,000-3,000 |
| C | 3,000-10,000 |
| D | 10,000-40,000 |

TABLE 4
Human FXIIa data, molecular weight and LCMS data

| Example | Human FXIIa | Molecular | LCMS |
|---|---|---|---|
| number | IC50 (nM) | weight | Mass Ion |
| 18.01 | B | 363.2 | 364.1 |
| 18.02 | D | 364.2 | 365.2 |
| 18.03 | A | 377.2 | 378.4 |
| 18.04 | A | 377.2 | 378.2 |
| 18.05 | A | 364.2 | 365 |
| 18.06 | B | 364.2 | 365.2 |
| 18.07 | B | 348.2 | 349.1 |
| 18.08 | A | 362.2 | 363.2 |
| 18.09 | D | 379.2 | 380.2 |
| 18.10 | A | 378.2 | 379.2 |
| 18.11 | A | 377.2 | 378.1 |
| 18.12 | A | 377.2 | 378.3 |
| 18.13 | C | 404.2 | 405.5 |
| 18.14 | B | 377.2 | 378.5 |
| 18.15 | A | 406.2 | 407.2 |
| 18.16 | D | 390.2 | 391.6 |
| 18.17 | A | 420.3 | 421.2 |
| 18.18 | A | 376.5 | 377.2 |
| 18.19 | C | 363.5 | 364.4 |
| 18.20 | | 407.2 | 408.2 |
| 18.21 | | 391.3 | 392.2 |
| 18.201 | C | 404.2 | 405.2 |
| 18.202 | A | 363.2 | 364.2 |
| 18.203 | D | 363.2 | 364.1 |
| 18.204 | C | 390.2 | 391.2 |
| 18.205 | D | 376.2 | 377.1 |
| 18.206 | B | 377.2 | 378.2 |
| 18.207 | D | 364.2 | 365.1 |
| 18.208 | D | 379.2 | 380.4 |
| 18.209 | B | 364.2 | 365.1 |
| 18.210 | B | 363.2 | 364.4 |
| 18.211 | B | 362.2 | 363.4 |
| 18.212 | B | 363.2 | 364.4 |
| 18.213 | C | 349.4 | 350.1 |
| 18.214 | | 393.2 | 394.5 |
| 18.215 | | 363.2 | 364.1 |

### Determination of the % Inhibition for FXIa

FXIa inhibitory activity in vitro was determined using standard published methods (see e.g. Johansen et al., Int. J. Tiss. Reac. 1986, 8, 185; Shori et al., Biochem. Pharmacol., 1992, 43, 1209; Stürzebecher et al., Biol. Chem. Hoppe-Seyler, 1992, 373, 1025). Human FXIa (Enzyme Research Laboratories) was incubated at 25° C. with the fluorogenic substrate Z-Gly-Pro-Arg-AFC and various concentrations of the test compound. Residual enzyme activity (initial rate of reaction) was determined by measuring the change in fluorescence at 410 nm and the IC50 value for the test compound was determined.

TABLE 5
Selectivity; FXIa data

| Ex. No. | Human FXIa IC50 (nM) |
|---|---|
| 18.01 | 36400 |
| 18.03 | >40000 |
| 18.04 | >40000 |
| 18.05 | 19900 |
| 18.08 | >40000 |
| 18.10 | 24800 |
| 18.11 | 17900 |
| 18.12 | 33100 |
| 18.202 | >40000 |

### NUMBERED EMBODIMENTS

1. A compound of formula (I),

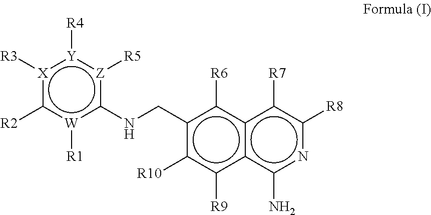

wherein:
W, X, Y, and Z are independently selected from C and N such that the ring containing W, X, Y, and Z is selected from benzene, pyridine, pyridazine, pyrimidine, pyrazine, and triazine;
R1, R4 and R5 are independently absent, or independently selected from H, alkyl, alkoxy, —CF₃, —OH, —CN, halo, —COOR12, and —CONR14R15;
when X is C, one of R2 and R3 is —L-V-R13, and the other of R2 and R3 is selected from H, alkyl, alkoxy, —CF₃, —OH, —CN, halo, —COOR12, and —CONR14R15; or when X is N, R2 is —L-V-R13, and R3 is absent;
R6, R7, R8, R9, and R10 are independently selected from H, alkyl, alkoxy,—CF₃, —OH, —CN, halo, —COOR12, and —CONR14R15;
L is selected from a bond, alkylene, and —C(O)—;
V is absent, or selected from O and NR12;
R12 is selected from H and alkyl^{b};
R13 is (CH₂)₀₋₃(heterocyclyl);

alkyl is a linear saturated hydrocarbon having up to 4 carbon atoms (C₁-C₄) or a branched saturated hydrocarbon of 3 or 4 carbon atoms (C₃-C₄); alkyl may optionally be substituted with 1 or 2 substituents independently selected from (C₁-C₃)alkoxy, —OH, —CN, —NR14R15, —NHCOCH₃, halo, —COOR12, and —CONR14R15;

alkyl^{b} is a linear saturated hydrocarbon having up to 4 carbon atoms (C₁-C₄) or a branched saturated hydrocarbon of 3 or 4 carbon atoms (C₃-C₄); alkyl^{b} may optionally be substituted with 1 or 2 substituents independently selected from —OH, —CN, —NHCOCH₃, and halo;

alkylene is a bivalent linear saturated hydrocarbon having 1 to 4 carbon atoms (C₁-C₄) or a branched bivalent saturated hydrocarbon having 3 to 4 carbon atoms (C3-C4);

alkoxy is a linear O-linked hydrocarbon of between 1 and 3 carbon atoms (C₁-C₃) or a branched O-linked hydrocarbon of between 3 and 4 carbon atoms (C₃-C₄); alkoxy may optionally be substituted with 1 or 2 substituents independently selected from —OH, —CN, —CF₃, —N(R12)₂ and fluoro;

halo is F, CI, Br, or I;

heterocyclyl is a 4-, 5-, or 6-, membered carbon-containing non-aromatic ring containing one or two ring members that are selected from N, NR16, and O; l heterocyclyl may be optionally substituted with 1, 2, 3, or 4 substituents independently selected from alkyl, alkoxy, oxo, —CF₃, —H, —CN, halo, —COOR12, and —CONR14R15;

R14 and R15 are independently selected from H, and alkyl^{b};

R16 is selected from H, and alkyl;

and tautomers, isomers, stereoisomers (including enantiomers, diastereoisomers and racemic and scalemic mixtures thereof), deuterated isotopes, and pharmaceutically acceptable salts and/or solvates thereof.

2. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein W, X, Y, and Z are independently selected from C and N such that the ring containing W, X, Y, and Z is benzene.
   3. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein W, X, Y, and Z are independently selected from C and N such that the ring containing W, X, Y, and Z is pyridine.
   4. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein W, X, Y, and Z are independently selected from C and N such that the ring containing W, X, Y, and Z is pyridazine.
   5. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein W, X, Y, and Z are independently selected from C and N such that the ring containing W, X, Y, and Z is pyrimidine.
   6. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein W, X, Y, and Z are independently selected from C and N such that the ring containing W, X, Y, and Z is pyrazine.
   7. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein W, X, Y, and Z are independently selected from C and N such that the ring containing W, X, Y, and Z is triazine.
   8. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein W is N and R1 is absent.
   9. A compound of formula (I) according to any of numbered embodiments 1 to 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is H.
   10. A compound of formula (I) according to any of numbered embodiments 1 to 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is alkyl.
   11. A compound of formula (I) according to numbered embodiment 10, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is methyl.
   12. A compound of formula (I) according to numbered embodiment 10, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is ethyl.
   13. A compound of formula (I) according to any of numbered embodiments 10 to 12, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is substituted with —OH.
   14. A compound of formula (I) according to any of numbered embodiments 10 to 12, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is substituted with —OMe.
   15. A compound of formula (I) according to any of numbered embodiments 1 to 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is alkoxy.
   16. A compound of formula (I) according to any of numbered embodiments 1 to 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —CF₃.
   17. A compound of formula (I) according to any of numbered embodiments 1 to 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —OH.
   18. A compound of formula (I) according to any of numbered embodiments 1 to 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —CN.
   19. A compound of formula (I) according to any of numbered embodiments 1 to 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is halo.
   20. A compound of formula (I) according to any of numbered embodiments 1 to 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —COOR12.
   21. A compound of formula (I) according to any of numbered embodiments 1 to 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —CONR14R15.
   22. A compound of formula (I) according to numbered embodiment 21, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —CONH₂.
   23. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is N and R4 is absent.
   24. A compound of formula (I) according to any of numbered embodiments 1 to 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.
   25. A compound of formula (I) according to any of numbered embodiments 1 to 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.
   26. A compound of formula (I) according to numbered embodiment 25, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is methyl.
   27. A compound of formula (I) according to numbered embodiment 25, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is ethyl.
   28. A compound of formula (I) according to any of numbered embodiments 25 to 27, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is substituted with —OH.
   29. A compound of formula (I) according to any of numbered embodiments 25 to 27, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is substituted with —OMe.
   30. A compound of formula (I) according to any of numbered embodiments 1 to 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkoxy.
   31. A compound of formula (I) according to any of numbered embodiments 1 to 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is —CF₃.
   32. A compound of formula (I) according to any of numbered embodiments 1 to 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is —OH.
   33. A compound of formula (I) according to any of numbered embodiments 1 to 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is —CN.
   34. A compound of formula (I) according to any of numbered embodiments 1 to 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is halo.
   35. A compound of formula (I) according to any of numbered embodiments 1 to 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is —COOR12.
   36. A compound of formula (I) according to any of numbered embodiments 1 to 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is —CONR14R15.
   37. A compound of formula (I) according to numbered embodiment 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is —CONH₂.
   38. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is N and R5 is absent.
   39. A compound of formula (I) according to any of numbered embodiments 1 to 37, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is H.
   40. A compound of formula (I) according to any of numbered embodiments 1 to 37, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is alkyl.
   41. A compound of formula (I) according to numbered embodiment 40, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is methyl.
   42. A compound of formula (I) according to numbered embodiment 40, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is ethyl.
   43. A compound of formula (I) according to any of numbered embodiments 40 to 42, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is substituted with —OH.
   44. A compound of formula (I) according to any of numbered embodiments 40 to 42, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is substituted with —OMe.
   45. A compound of formula (I) according to any of numbered embodiments 1 to 37, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is alkoxy.
   46. A compound of formula (I) according to any of numbered embodiments 1 to 37, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —CF₃.
   47. A compound of formula (I) according to any of numbered embodiments 1 to 37, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —OH.
   48. A compound of formula (I) according to any of numbered embodiments 1 to 37, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —CN.
   49. A compound of formula (I) according to any of numbered embodiments 1 to 37, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is halo.
   50. A compound of formula (I) according to any of numbered embodiments 1 to 37, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —COOR12.
   51. A compound of formula (I) according to any of numbered embodiments 1 to 37, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —CONR14R15.
   52. A compound of formula (I) according to numbered embodiment 51, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —CONH₂.
   53. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is C and R2 is —L-V-R13.
   54. A compound of formula (I) according to any of numbered embodiments 1 to 52, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is C and R3 is —L-V-R13.
   55. A compound of formula (I) according to any of numbered embodiments 1 to 52, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is N, R2 is —L-V-R13, and R3 is absent.
   56. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein W is C.
   57. A compound of formula (I) according to any of numbered embodiments 1 to 7, and 23 to 55, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein W is N and R1 is absent.
   58. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is C.
   59. A compound of formula (I) according to any of numbered embodiments 1 to 22, and 38 to 57, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is N and R4 is absent.
   60. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is C.
   61. A compound of formula (I) according to any of numbered embodiments 1 to 37, and 53 to 59, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is N and R5 is absent.
   62. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the one of R2 or R3 that is not —L-V-R13 is H.
   63. A compound of formula (I) according to any of numbered embodiments 1 to 61, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the one of R2 or R3 that is not —L-V-R13 is alkyl.
   64. A compound of formula (I) according to any of numbered embodiments 1 to 61, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the one of R2 or R3 that is not —L-V-R13 is alkoxy.
   65. A compound of formula (I) according to any of numbered embodiments 1 to 61, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the one of R2 or R3 that is not —L-V-R13 is —CF₃.
   66. A compound of formula (I) according to any of numbered embodiments 1 to 61, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the one of R2 or R3 that is not —L-V-R13 is —OH.
   67. A compound of formula (I) according to any of numbered embodiments 1 to 61, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the one of R2 or R3 that is not —L-V-R13 is —CN.
   68. A compound of formula (I) according to any of numbered embodiments 1 to 61, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the one of R2 or R3 that is not —L-V-R13 is halo.
   69. A compound of formula (I) according to any of numbered embodiments 1 to 61, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the one of R2 or R3 that is not —L-V-R13 is —COOR12.
   70. A compound of formula (I) according to any of numbered embodiments 1 to 61, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the one of R2 or R3 that is not —L-V-R13 is —CONR14R15.
   71. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein L is a bond.
   72. A compound of formula (I) according to any of numbered embodiments 1 to 70, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein L is alkylene.
   73. A compound of formula (I) according to numbered embodiment 72, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein L is methylene.
   74. A compound of formula (I) according to any of numbered embodiments 1 to 70, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein L is —C(O)—.
   75. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein V is absent.
   76. A compound of formula (I) according to any of numbered embodiments 1 to 74, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein V is O.
   77. A compound of formula (I) according to any of numbered embodiments 1 to 74, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein V is NR12.
   78. A compound of formula (I) according to numbered embodiment 77, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R12 is H.
   79. A compound of formula (I) according to numbered embodiment 77, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R12 is alkyl^{b}.
   80. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R13 is heterocyclyl.
   81. A compound of formula (I) according to any of numbered embodiments 1 to 79, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R13 is —CH₂-heterocyclyl.
   82. A compound of formula (I) according to any of numbered embodiments 1 to 79, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R13 is —(CH₂)₂-heterocyclyl.
   83. A compound of formula (I) according to any of numbered embodiments 1 to 79, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R13 is —(CH₂)₃-heterocyclyl.
   84. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is a 4-membered carbon-containing non-aromatic ring containing one or two ring members that are selected from N, NR16, and O; heterocyclyl may be optionally substituted with 1, 2, 3, or 4 substituents independently selected from alkyl, alkoxy, oxo, —CF₃, —OH, —CN, halo, —COOR12, and —CONR14R15.
   85. A compound of formula (I) according to numbered embodiment 84, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is azetidinyl, which may be optionally substituted as for heterocyclyl.
   86. A compound of formula (I) according to numbered embodiment 84, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is oxetanyl, which may be optionally substituted as for heterocyclyl.
   87. A compound of formula (I) according to any of numbered embodiments 1 to 83, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is a 5-membered carbon-containing non-aromatic ring containing one or two ring members that are selected from N, NR16, and O; heterocyclyl may be optionally substituted with 1, 2, 3, or 4 substituents independently selected from alkyl, alkoxy, oxo, —CF₃, —OH, —CN, halo, —COOR12, and —CONR14R15.
   88. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is pyrrolidinyl, which may be optionally substituted as for heterocyclyl.
   89. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is tetrahydrofuranyl, which may be optionally substituted as for heterocyclyl.
   90. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is pyrazolidinyl, which may be optionally substituted as for heterocyclyl.
   91. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is imidazolidinyl, which may be optionally substituted as for heterocyclyl.
   92. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is 3-dioxolanyl, which may be optionally substituted as for heterocyclyl.
   93. A compound of formula (I) according to any of numbered embodiments 1 to 83, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is a 6-membered carbon-containing non-aromatic ring containing one or two ring members that are selected from N, NR16, and 0; heterocyclyl may be optionally substituted with 1, 2, 3, or 4 substituents independently selected from alkyl, alkoxy, oxo, —CF₃, —OH, —CN, halo, —COOR12, and —CONR14R15.
   94. A compound of formula (I) according to numbered embodiment 93, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is piperidinyl, which may be optionally substituted as for heterocyclyl.
   95. A compound of formula (I) according to numbered embodiment 93, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is piperazinyl, which may be optionally substituted as for heterocyclyl.
   96. A compound of formula (I) according to numbered embodiment 93, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is morpholinyl, which may be optionally substituted as for heterocyclyl.
   97. A compound of formula (I) according to numbered embodiment 93, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is 1,4-dioxanyl, which may be optionally substituted as for heterocyclyl.
   98. A compound of formula (I) according to numbered embodiment 93, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is piperidinyl, which may be optionally substituted as for heterocyclyl.
   99. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R13 is substituted with oxo.
   100. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when present, NR16 is NH.
   101. A compound of formula (I) according to any of numbered embodiments 1 to 99, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when present, NR16 is N(alkyl).
   102. A compound of formula (I) according to numbered embodiment 101, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when present, NR16 is NCH₃.
   103. A compound of formula (I) according to numbered embodiment 101, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when present, NR16 is NCH₂CH₃.
   104. A compound of formula (I) according to numbered embodiment 101, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when present, NR16 is NCH₂CH₂OH.
   105. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is H.
   106. A compound of formula (I) according to any of numbered embodiments 1 to 104, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is alkyl.
   107. A compound of formula (I) according to any of numbered embodiments 1 to 104, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is alkoxy.
   108. A compound of formula (I) according to any of numbered embodiments 1 to 104, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is —CF₃.
   109. A compound of formula (I) according to any of numbered embodiments 1 to 104, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is —OH.
   110. A compound of formula (I) according to any of numbered embodiments 1 to 104, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is —CN.
   111. A compound of formula (I) according to any of numbered embodiments 1 to 104, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is halo.
   112. A compound of formula (I) according to any of numbered embodiments 1 to 104, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is —COOR12.
   113. A compound of formula (I) according to any of numbered embodiments 1 to 104, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is —CONR14R15.
   114. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R7 is H.
   115. A compound of formula (I) according to any of numbered embodiments 1 to 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R7 is alkyl.
   116. A compound of formula (I) according to any of numbered embodiments 1 to 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R7 is alkoxy.
   117. A compound of formula (I) according to any of numbered embodiments 1 to 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R7 is —CF₃.
   118. A compound of formula (I) according to any of numbered embodiments 1 to 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R7 is —OH.
   119. A compound of formula (I) according to any of numbered embodiments 1 to 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R7 is —CN.
   120. A compound of formula (I) according to any of numbered embodiments 1 to 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R7 is halo.
   121. A compound of formula (I) according to any of numbered embodiments 1 to 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R7 is —COOR12.
   122. A compound of formula (I) according to any of numbered embodiments 1 to 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R7 is —CONR14R15.
   123. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R8 is H.
   124. A compound of formula (I) according to any of numbered embodiments 1 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R8 is alkyl.
   125. A compound of formula (I) according to any of numbered embodiments 1 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R8 is alkoxy.
   126. A compound of formula (I) according to any of numbered embodiments 1 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R8 is —CF₃.
   127. A compound of formula (I) according to any of numbered embodiments 1 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R8 is —OH.
   128. A compound of formula (I) according to any of numbered embodiments 1 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R8 is —CN.
   129. A compound of formula (I) according to any of numbered embodiments 1 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R8 is halo.
   130. A compound of formula (I) according to any of numbered embodiments 1 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R8 is —COOR12.
   131. A compound of formula (I) according to any of numbered embodiments 1 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R8 is —CONR14R15.
   132. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R9 is H.
   133. A compound of formula (I) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R9 is alkyl.
   134. A compound of formula (I) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R9 is alkoxy.
   135. A compound of formula (I) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R9 is —CF₃.
   136. A compound of formula (I) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R9 is —OH.
   137. A compound of formula (I) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R9 is —CN.
   138. A compound of formula (I) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R9 is halo.
   139. A compound of formula (I) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R9 is —COOR12.
   140. A compound of formula (I) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R9 is —CONR14R15.
   141. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R10 is H.
   142. A compound of formula (I) according to any of numbered embodiments 1 to 140, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R10 is alkyl.
   143. A compound of formula (I) according to any of numbered embodiments 1 to 140, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R10 is alkoxy.
   144. A compound of formula (I) according to any of numbered embodiments 1 to 140, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R10 is —CF₃.
   145. A compound of formula (I) according to any of numbered embodiments 1 to 140, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R10 is —OH.
   146. A compound of formula (I) according to any of numbered embodiments 1 to 140, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R10 is —CN.
   147. A compound of formula (I) according to any of numbered embodiments 1 to 140, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R10 is halo.
   148. A compound of formula (I) according to any of numbered embodiments 1 to 140, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R10 is —COOR12.
   149. A compound of formula (I) according to any of numbered embodiments 1 to 140, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R10 is —CONR14R15.
   150. A compound selected from Table 1 or Table 2, or a pharmaceutically acceptable salt, solvate, or solvate of a salt thereof.
   151. A compound according to any preceding numbered embodiment.
   152. A pharmaceutically acceptable salt according to any of numbered embodiments 1 to 151.
   153. A pharmaceutically acceptable solvate according to any of numbered embodiments 1 to 151.
   154. A pharmaceutically acceptable solvate of a salt according to any of numbered embodiments 1 to 151.
   155. A pharmaceutical composition comprising:
(i) a compound according to numbered embodiment 151, a pharmaceutically acceptable salt according to numbered embodiment 152, a pharmaceutically acceptable solvate according to numbered embodiment 153, or a pharmaceutically acceptable solvate of a salt according to numbered embodiment 154; and
(ii) at least one pharmaceutically acceptable excipient.
   156. A compound as defined in numbered embodiment 151, a pharmaceutically acceptable salt according to numbered embodiment 152, a pharmaceutically acceptable solvate according to numbered embodiment 153, a pharmaceutically acceptable solvate of a salt according to numbered embodiment
   154, or a pharmaceutical composition as defined in numbered embodiment 155, for use in medicine.
   157. The use of a compound as defined in numbered embodiment 151, a pharmaceutically acceptable salt according to numbered embodiment 152, a pharmaceutically acceptable solvate according to numbered embodiment 153, a pharmaceutically acceptable solvate of a salt according to numbered embodiment 154, or a pharmaceutical composition as defined in numbered embodiment 155, in the manufacture of a medicament for the treatment or prevention of a disease or condition in which Factor XIIa activity is implicated.
   158. A method of treatment of a disease or condition in which Factor XIIa activity is implicated comprising administration to a subject in need thereof a therapeutically effective amount of a compound as defined in numbered embodiment 151, a pharmaceutically acceptable salt according to numbered embodiment 152, a pharmaceutically acceptable solvate according to numbered embodiment 153, a pharmaceutically acceptable solvate of a salt according to numbered embodiment
   154 or a pharmaceutical composition as defined in numbered embodiment 155.
   159. A compound as defined in numbered embodiment 151, a pharmaceutically acceptable salt according to numbered embodiment 152, a pharmaceutically acceptable solvate according to numbered embodiment 153, a pharmaceutically acceptable solvate of a salt according to numbered embodiment
   154, or a pharmaceutical composition as defined in numbered embodiment 155, for use in a method of treatment of a disease or condition in which Factor XIIa activity is implicated.
   160. The use of numbered embodiment 157, the method of numbered embodiment 158, or a compound, a pharmaceutically acceptable salt, a pharmaceutically acceptable solvate, a pharmaceutically acceptable solvate of a salt, or a pharmaceutical composition for use as defined in numbered embodiment 159, wherein, the disease or condition in which Factor XIIa activity is implicated is a bradykinin-mediated angioedema.
   161. The use of numbered embodiment 160, the method of numbered embodiment 160, or a compound, a pharmaceutically acceptable salt, a pharmaceutically acceptable solvate, a pharmaceutically acceptable solvate of a salt, or a pharmaceutical composition for use as defined in numbered embodiment 160, wherein the bradykinin-mediated angioedema is hereditary angioedema.
   162. The use of numbered embodiment 160, the method of numbered embodiment 160, or a compound, a pharmaceutically acceptable salt, a pharmaceutically acceptable solvate, a pharmaceutically acceptable solvate of a salt, or a pharmaceutical composition for use as defined in numbered embodiment 160, wherein the bradykinin-mediated angioedema is non hereditary.
   163. The use of numbered embodiment 157, the method of numbered embodiment 158, or a compound, a pharmaceutically acceptable salt, a pharmaceutically acceptable solvate, a pharmaceutically acceptable solvate of a salt, or a pharmaceutical composition for use as defined in numbered embodiment 159, wherein the disease or condition in which Factor XIIa activity is implicated is selected from vascular hyperpermeability, stroke including ischemic stroke and haemorrhagic accidents; retinal edema; diabetic retinopathy; DME; retinal vein occlusion; and AMD.
   164. The use of numbered embodiment 157, the method of numbered embodiment 158, or a compound, a pharmaceutically acceptable salt, a pharmaceutically acceptable solvate, a pharmaceutically acceptable solvate of a salt, or a pharmaceutical composition for use as defined in numbered embodiment 159, wherein, the disease or condition in which Factor XIIa activity is implicated is a thrombotic disorder.
   165. The use of numbered embodiment 164, the method of numbered embodiment 164, or a compound, a pharmaceutically acceptable salt, a pharmaceutically acceptable solvate, a pharmaceutically acceptable solvate of a salt, or a pharmaceutical composition for use as defined in numbered embodiment 164, wherein the thrombotic disorder is thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget Schroetter syndrome and Budd-Chari syndrome; and atherosclerosis.
   166. The use of numbered embodiment 157, the method of numbered embodiment 158, or a compound, a pharmaceutically acceptable salt, a pharmaceutically acceptable solvate, a pharmaceutically acceptable solvate of a salt, or a pharmaceutical composition for use as defined in numbered embodiment 159, wherein, the disease or condition in which Factor XIIa activity is implicated is selected from neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.
   167. The use of any of numbered embodiments 157 or 160 to 166, the method of any of numbered embodiments 158 or 160 to 166, or a compound, a pharmaceutically acceptable salt, a pharmaceutically acceptable solvate, a pharmaceutically acceptable solvate of a salt, or a pharmaceutical composition for use as defined in any of numbered embodiments 159 or 160 to 166, wherein the compound targets FXIIa.

1. A compound of formula (I): 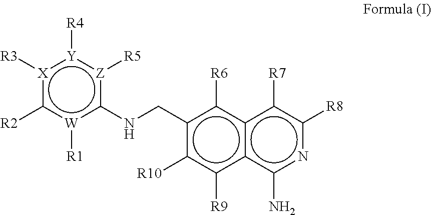
wherein.
W, X, Y, and Z are independently selected from C or N such that the ring containing W, X, Y, and Z is selected from benzene, pyridine, pyridazine, pyrimidine, pyrazine, or triazine;
R1, R4 and R5 are, independently, absent, H, alkyl, alkoxy, —CF₃, —OH, —CN, halo, —COOR12, or —CONR14R15;
when X is C, one of R2 and R3 is —L-V-R13, and the other of R2 and R3 is selected from alkyl, alkoxy, —CF₃, —OH, —CN, halo, —COOR12, or —CONR14R15; or when X is N, R2 is —L-V-R13, and R3 is absent;
R6, R R8, R9, and R10 are independently selected from H, alkyl, alkoxy, —CF₃, —OH, —CN, halo, —COOR12, or —CONR14R15;
L is selected from a bond, alkylene, or —C(O)—;
V is absent, O or NR12;
R12 is selected from H or alkyl^{b};
R13 is (CH₂)₀₋₃(heterocyclyl);
alkyl is a linear saturated hydrocarbon having up to 4 carbon atoms (C₁-C₄) or a branched saturated hydrocarbon of 3 or 4 carbon atoms (C₃-C₄); alkyl is optionally substituted with 1 or 2 substituents independently selected from (C₁-C₃)alkoxy, —OH, —CN, —NR14R15, —NHCOCH₃, halo, —COOR12, or —CONR14R15;
alkyl^{b} is a linear saturated hydrocarbon having up to 4 carbon atoms (C1-C4) or a branched saturated hydrocarbon of 3 or 4 carbon atoms (C3-C4); alkyl^{b} is optionally substituted with 1 or 2 substituents independently selected from —OH, —CN, —NHCOCH_{3,} or halo;
alkylene is a bivalent linear saturated hydrocarbon having 1 to 4 carbon atoms (C₁-C₄) or a branched bivalent saturated hydrocarbon having 3 to 4 carbon atoms (C₃-C₄);
alkoxy is a linear O-linked hydrocarbon of between 1 and 3 carbon atoms (C₁-C₃) or a branched O-linked hydrocarbon of between 3 and 4 carbon atoms (C₃-C₄); alkoxy is optionally substituted with 1 or 2 substituents independently selected from —OH, —CN, —CF_{3,} —N(R12)₂ or fluoro;
halo is F, CI, Br, or I;
heterocyclyl is a 4-, 5-, or 6-, membered carbon-containing non-aromatic ring containing one or two ring members that are selected from N, NR16, or O; heterocyclyl is optionally substituted with 1, 2, 3, or 4 substituents independently selected from alkyl, alkoxy, oxo, —CF_{3,} —OH, —CN, halo, —COOR12, or —CONR14R15;
R14 and R15 are independently selected from H or alkyl^{b};
R16 is selected from H or alkyl;
or a tautomer, isomer, stereoisomer, deuterated isotope, pharmaceutically acceptable salt and/or solvate thereof.

2. The compound of formula (I) according to claim 1, or a tautomer, isomer, stereoisomer, deuterated isotope, pharmaceutically acceptable salt and/or solvate thereof, wherein W, Y and Z are independently selected from C or N such that the ring containing W, X, Y and Z is selected from benzene, pyridine or pyridazine.

3. The compound of formula (I) according to claim 1, or a tautomer, isomer, stereoisomer, a deuterated isotope, a pharmaceutically acceptable salt and/or solvate thereof, wherein W, X, Y and Z are independently selected from C or N such that the ring containing W, X, Y and Z is benzene.

4. The compound of formula (I) according to claim 1, or a tautomer, isomer, stereoisomer, a deuterated isotope, a pharmaceutically acceptable salt and/or solvate thereof, wherein W, X, Y and Z are independently selected from C or N such that the ring containing W, X, Y and Z is pyridine.

5. The compound of formula (I) according to claim 1, or a tautomer, isomer, stereoisomer, a deuterated isotope, a pharmaceutically acceptable salt and/or solvate thereof, wherein W, X, Y and Z are independently selected from C or N such that the ring containing W X, Y and Z is pyrazine.

6. The compound of formula (I) according to claim 3, or a tautomer, isomer, stereoisomer, a deuterated isotope, a pharmaceutically acceptable salt and/or solvate thereof, wherein R1, R4 and R5 are H.

7. The compound of formula (I) according to claim 4, or a tautomer, isomer, stereoisomer, a deuterated isotope, a pharmaceutically acceptable salt and/or solvate thereof, wherein one of R1, R4 and R5 is absent and the other two are H.

8. The compound of formula (I) according to claim 5, or a tautomer, isomer, stereoisomer, a deuterated isotope, a pharmaceutically acceptable salt and/or solvate thereof, wherein two of R1, R4 and R5 is absent and the other one is H.

9. The compound of formula (I) according to claim 1, or a tautomer, isomer, stereoisomer, a deuterated isotope, a pharmaceutically acceptable salt and/or solvate thereof, wherein X is C.

10. The compound of formula (I) according to claim 9, or a tautomer, isomer, stereoisomer, a deuterated isotope, a pharmaceutically acceptable salt and/or solvate thereof, wherein R2 is selected from H, alkyl, alkoxy, —CF₃, —OH, —CN, halo, —COOR12, or —CONR14R15 and R3 is —L-V-R13.

11. The compound of formula (I) according to claim 9, or a tautomer, isomer, stereoisomer, a deuterated isotope, a pharmaceutically acceptable salt and/or solvate thereof, wherein R3 is selected from H, alkyl, alkoxy, —CF₃, —OH, —CN, halo, —COOR12, or —CONR14R15 and R2 is —L-V-R13. 12, The compound of formula (1) according to claim 1, or a tautomer, isomer, stereoisomer, a deuterated isotope, a pharmaceutically acceptable salt and/or solvate thereof, wherein L is a bond.

13. The compound of formula (I) according to claim 1, or a tautomer, isomer, stereoisomer, a deuterated isotope, a pharmaceutically acceptable salt and/or solvate thereof, wherein L is methylene.

14. The compound of formula (I) according to claim 1, or a tautomer, isomer, stereoisomer, a deuterated isotope, a pharmaceutically acceptable salt and/or solvate thereof, wherein V is O.

15. The compound of formula (I) according to claim 1, or a tautomer, isomer, stereoisomer, a deuterated isotope, a pharmaceutically acceptable salt and/or solvate thereof, wherein R13 is heterocyclyl.

16. The compound of formula (I) according to claim 1, or a tautomer, isomer, stereoisomer, a deuterated isotope, a pharmaceutically acceptable salt and/or solvate thereof, wherein R13 is —CH₂-heterocyclyl.

17. The compound of formula (I) according to claim 1, or a tautomer, isomer, stereoisomer, a deuterated isotope, a pharmaceutically acceptable salt and/or solvate thereof, wherein the heterocyclyl on R13 is piperidinyl, which is optionally substituted as for heterocyclyl.

18. The compound of formula (I) according to claim 1, or a tautomer, isomer, stereoisomer, a deuterated isotope, a pharmaceutically acceptable salt and/or solvate thereof, wherein, when present, NR16 is NCH₃.

19. The compound of formula (I) according to claim 1, or a tautomer, isomer, stereoisomer, a deuterated isotope, a pharmaceutically acceptable salt and/or solvate thereof, wherein R6, R7, RS, R9 and R10 are all H.

20. A compound that is: 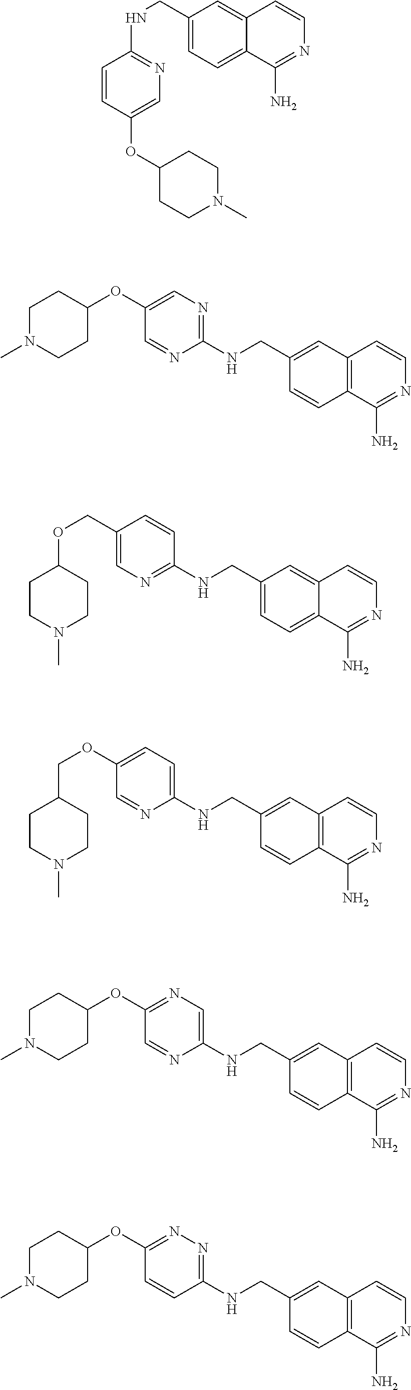 [SMILES: CN1CCC(Oc2ccc(NCc3ccc4c(N)nccc4c3)nc2)CC1 ; CN1CCC(Oc2cnc(NCc3ccc4c(N)nccc4c3)nc2)CC1 ; CN1CCC(OCc2ccc(NCc3ccc4c(N)nccc4c3)nc2)CC1 ; CN1CCC(COc2ccc(NCc3ccc4c(N)nccc4c3)nc2)CC1 ; CN1CCC(Oc2cnc(NCc3ccc4c(N)nccc4c3)cn2)CC1 ; CN1CCC(Oc2ccc(NCc3ccc4c(N)nccc4c3)nn2)CC1] 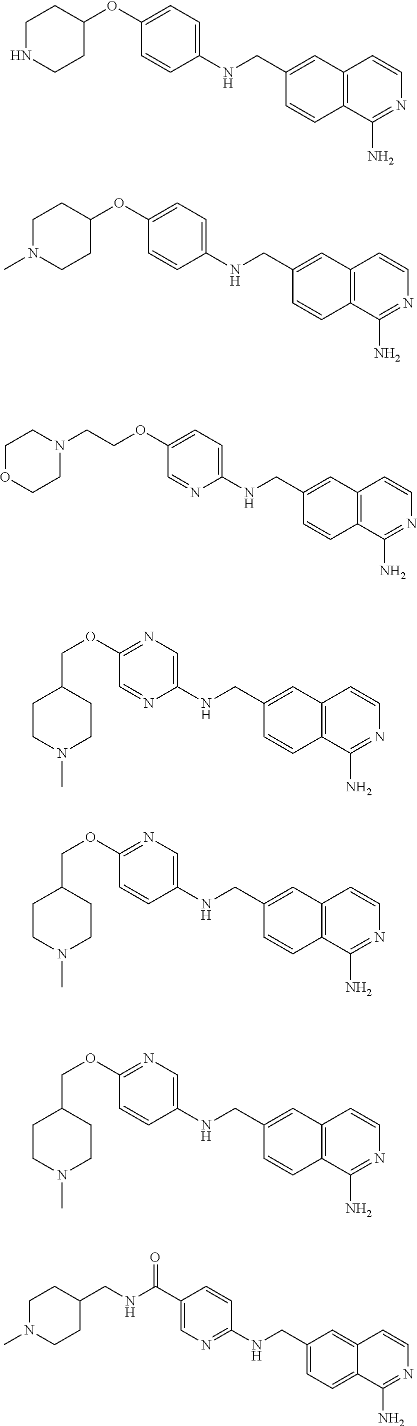 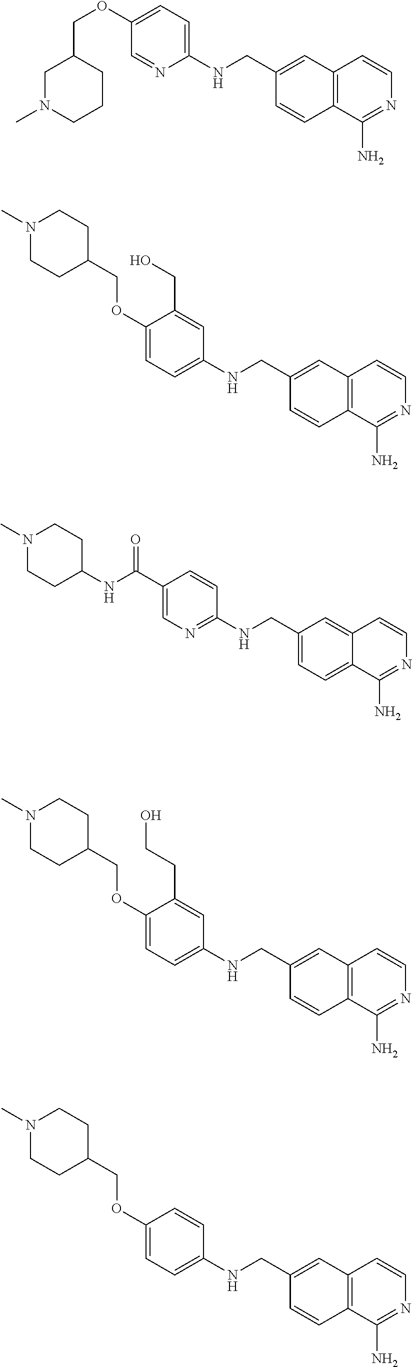 [SMILES: CN1CCCC(COc2ccc(NCc3ccc4c(N)nccc4c3)nc2)C1 ; CN1CCC(COc2ccc(NCc3ccc4c(N)nccc4c3)cc2CO)CC1 ; CN1CCC(NC(=O)c2ccc(NCc3ccc4c(N)nccc4c3)nc2)CC1 ; CN1CCC(COc2ccc(NCc3ccc4c(N)nccc4c3)cc2CCO)CC1 ; CN1CCC(COc2ccc(NCc3ccc4c(N)nccc4c3)cc2)CC1] 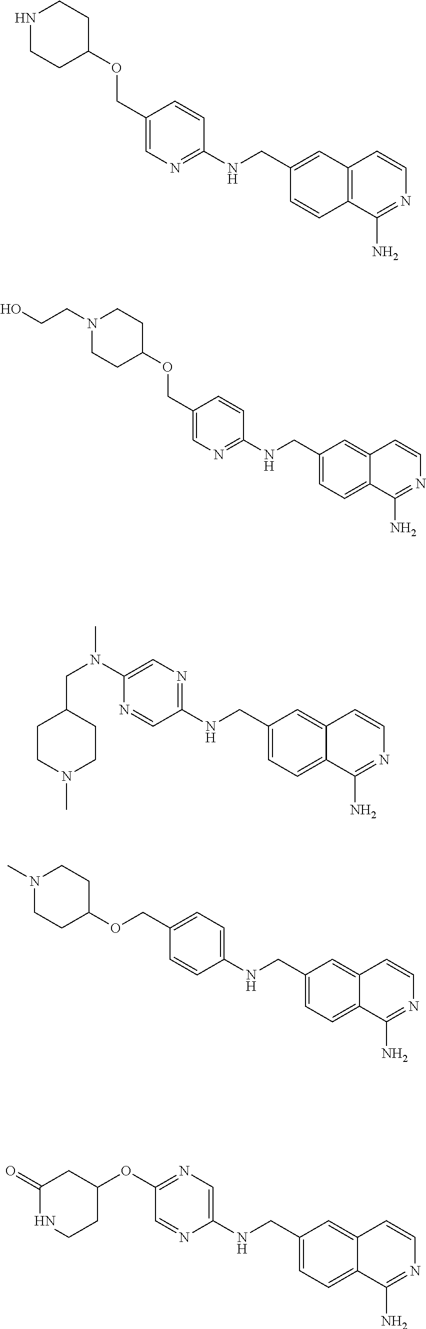 [SMILES: Nc1nccc2cc(CNc3ccc(COC4CCNCC4)cn3)ccc12 ; Nc1nccc2cc(CNc3ccc(COC4CCN(CCO)CC4)cn3)ccc12 ; CN1CCC(CN(C)c2cnc(NCc3ccc4c(N)nccc4c3)cn2)CC1 ; CN1CCC(OCc2ccc(NCc3ccc4c(N)nccc4c3)cc2)CC1 ; Nc1nccc2cc(CNc3cnc(OC4CCNC(=O)C4)cn3)ccc12] 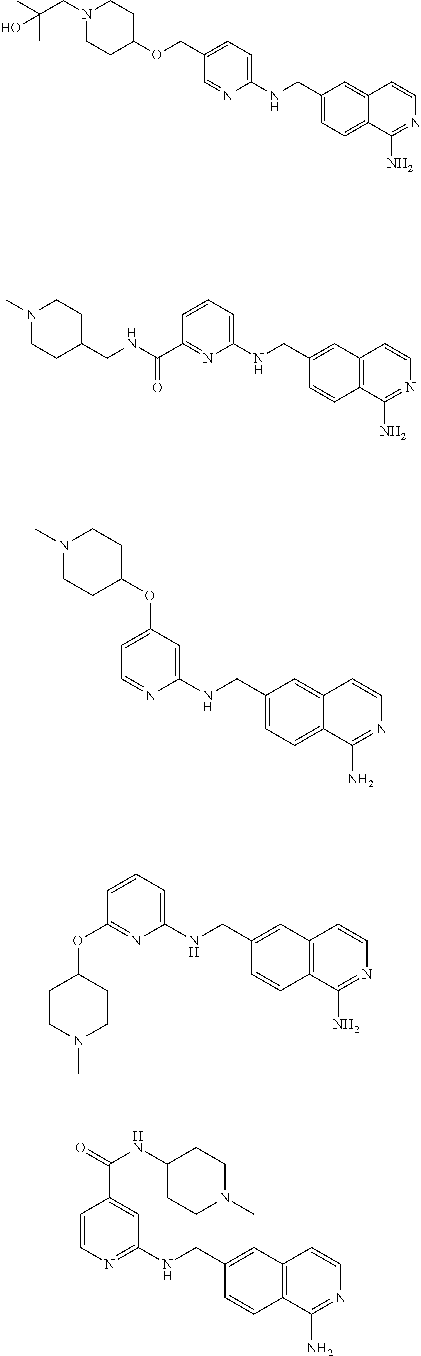 [SMILES: CC(C)(O)CN1CCC(OCc2ccc(NCc3ccc4c(N)nccc4c3)nc2)CC1 ; CN1CCC(CNC(=O)c2cccc(NCc3ccc4c(N)nccc4c3)n2)CC1 ; CN1CCC(Oc2ccnc(NCc3ccc4c(N)nccc4c3)c2)CC1 ; CN1CCC(Oc2cccc(NCc3ccc4c(N)nccc4c3)n2)CC1 ; CN1CCC(NC(=O)c2ccnc(NCc3ccc4c(N)nccc4c3)c2)CC1] 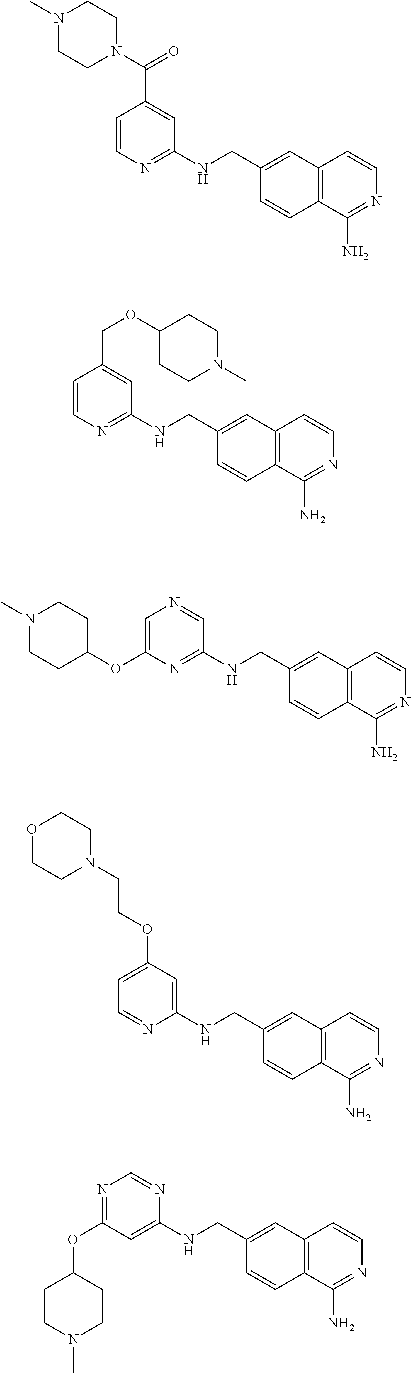 [SMILES: CN1CCN(C(=O)c2ccnc(NCc3ccc4c(N)nccc4c3)c2)CC1 ; CN1CCC(OCc2ccnc(NCc3ccc4c(N)nccc4c3)c2)CC1 ; CN1CCC(Oc2cncc(NCc3ccc4c(N)nccc4c3)n2)CC1 ; Nc1nccc2cc(CNc3cc(OCCN4CCOCC4)ccn3)ccc12 ; CN1CCC(Oc2cc(NCc3ccc4c(N)nccc4c3)ncn2)CC1] 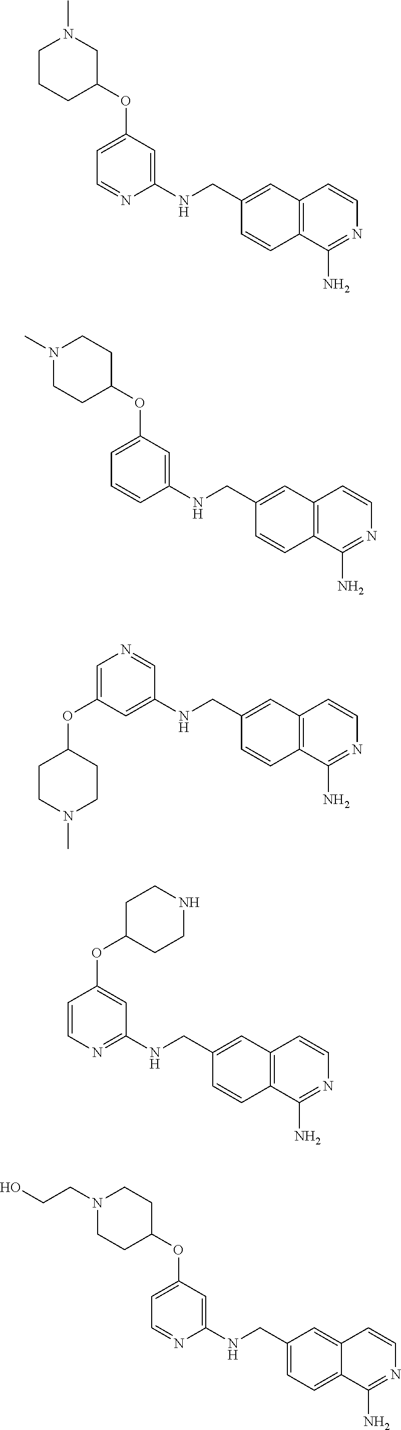 [SMILES: CN1CCCC(Oc2ccnc(NCc3ccc4c(N)nccc4c3)c2)C1 ; CN1CCC(Oc2cccc(NCc3ccc4c(N)nccc4c3)c2)CC1 ; CN1CCC(Oc2cncc(NCc3ccc4c(N)nccc4c3)c2)CC1 ; Nc1nccc2cc(CNc3cc(OC4CCNCC4)ccn3)ccc12 ; Nc1nccc2cc(CNc3cc(OC4CCN(CCO)CC4)ccn3)ccc12] 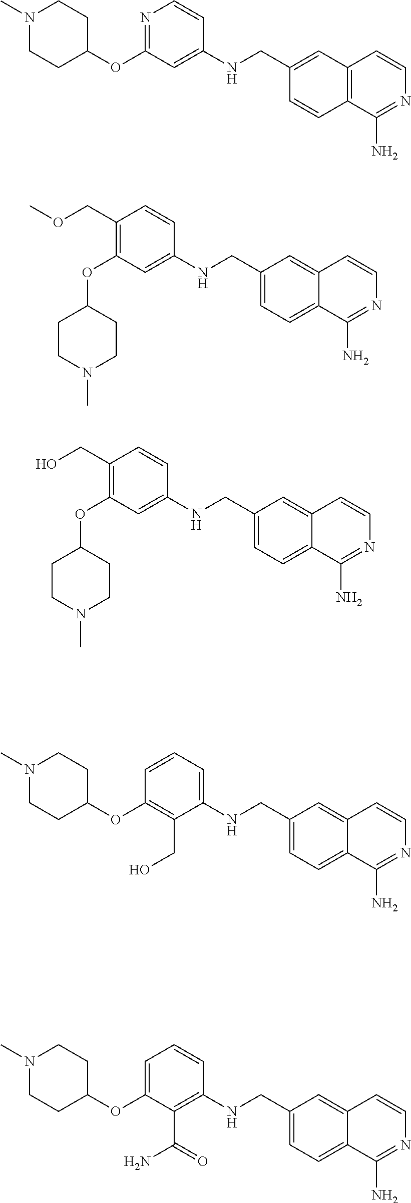
or a pharmaceutically acceptable salt, solvate, or solvate of a salt thereof.

21. A pharmaceutical composition comprising: a compound, or a pharmaceutically acceptable salt and/or solvate thereof, according to claim 1, and at least one pharmaceutically acceptable excipient.

22. (canceled)

23. (canceled)

24. A method of treating a disease or condition in which Factor XIIa activity is implicated, comprising administering to a subject in need thereof a. therapeutically effective amount of a compound, or a pharmaceutically acceptable salt and/or solvate thereof of claim 1.

25. (canceled)

26. The method of claim 24, wherein the disease or condition in which Factor XIIa activity is implicated is a bradykinin-mediated angioedema.

27. The method of claim 26, wherein the bradykinin-mediated angioedema is hereditary angioedema.

28. The method of claim 26, wherein the bradykinin-mediated angioedema is non hereditary.

29. The method of claim 24, wherein the disease or condition in which Factor Mk activity is implicated is selected from vascular hyperpermeability; stroke including ischemic stroke or haemorrhagic accidents; retinal edema; diabetic retinopathy; DME; retinal vein occlusion; or AMD,

30. The method of claim 24, wherein the disease or condition in which Factor XIIa activity is implicated is a thrombotic disorder.

31. The method of claim 30, wherein the thrombotic disorder is thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical or bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget Schroetter syndrome and Budd-Chari syndrome; or atherosclerosis.

32. The method of claim 24, wherein the disease or condition in which Factor XIIa activity is implicated is selected from neuroinflammation; neuroinflammatory/neurodegenerative disorders such as MS (multiple sclerosis); other neurodegenerative diseases such as Alzheimer's disease, epilepsy or migraine; sepsis; bacterial sepsis; inflammation; vascular hyperpermeability; or anaphylaxis.

33. The method of claim 24, wherein the compound targets FXIIa.

34. A method of treating a disease or condition in which Factor XIIa activity is implicated, comprising administering to a subject in need thereof a therapeutically effective amount of the pharmaceutical composition of claim 21.

35. The compound of claim 1, wherein the stereoisomer is an enantiomer, diastereoisomer, or a racemic or scalemic mixture thereof.
